SEQUENCE LISTING (425 sequences)

SEQ ID NO: 1 - RNA, 21 nt, Artificial Sequence
  Synthetic 177 RL2
ggaggccgccgaggacgucag

SEQ ID NO: 2 - RNA, 20 nt, Artificial Sequence
  Synthetic 098/350 RS1
gucaucgaccucgucggacu

SEQ ID NO: 3 - RNA, 21 nt, Artificial Sequence
  Synthetic 626 UL06
gucugucagguacguuugcag

SEQ ID NO: 4 - RNA, 21 nt, Artificial Sequence
  Synthetic 390 UL15
gcugcucgagcagauacucga

SEQ ID NO: 5 - RNA, 21 nt, Artificial Sequence
  Synthetic 411 UL19
gucgggcugcauaaacugcuc

SEQ ID NO: 6 - RNA, 21 nt, Artificial Sequence
  Synthetic 441 UL22
gacuucgaccggggucucgga

SEQ ID NO: 7 - RNA, 21 nt, Artificial Sequence
  Synthetic 480 UL29
gcgugauggacauguuuaaca

SEQ ID NO: 8 - RNA, 21 nt, Artificial Sequence
  Synthetic 504 UL32
gcccgucuccuggacugcacg

SEQ ID NO: 9 - RNA, 21 nt, Artificial Sequence
  Synthetic 514 UL33
gcgcugcggucccagacccug

SEQ ID NO: 10 - RNA, 22 nt, Artificial Sequence
  Synthetic 524 UL37
gccagaacguaggggacgaagc

SEQ ID NO: 11 - RNA, 22 nt, Artificial Sequence
  Synthetic 570 UL48
ggcgaggucgugaagcuggaau

SEQ ID NO: 12 - RNA, 21 nt, Artificial Sequence
  Synthetic 596 UL54
gcccuuugacgccgagaccag

SEQ ID NO: 13 - RNA, 20 nt, Artificial Sequence
  Synthetic 097 RL2
gaggccgccgaggacgucag

SEQ ID NO: 14 - RNA, 21 nt, Artificial Sequence
  Synthetic 099/571 UL48
gcgaggucgugaagcuggaau

SEQ ID NO: 15 - RNA, 21 nt, Artificial Sequence
  Synthetic 272 RL2
gcucaggccgcgaaccaagaa

SEQ ID NO: 16 - RNA, 23 nt, Artificial Sequence
  Synthetic 345 RS1
ggcggcggcccgucgguggggcc

SEQ ID NO: 17 - RNA, 20 nt, Artificial Sequence
  Synthetic 385 UL15
gugcguggcgauggcgacgg

SEQ ID NO: 18 - RNA, 22 nt, Artificial Sequence
  Synthetic 407 UL19
gccugcagcagguaaaacaccg

SEQ ID NO: 19 - RNA, 24 nt, Artificial Sequence
  Synthetic 466 UL22
ggugcgugauguccagcuccgucg

SEQ ID NO: 20 - RNA, 23 nt, Artificial Sequence
  Synthetic 479 UL29
gcgcgugauggacauguuuaaca

SEQ ID NO: 21 - RNA, 23 nt, Artificial Sequence
  Synthetic 503 UL32
gcgcccgucuccuggacugcacg

SEQ ID NO: 22 - RNA, 20 nt, Artificial Sequence
  Synthetic 535 UL37
gcacuaccuuucggcccugg

SEQ ID NO: 23 - RNA, 21 nt, Artificial Sequence
  Synthetic 551 UL37
guucuuggcccuggugaguaa

SEQ ID NO: 24 - RNA, 22 nt, Artificial Sequence
  Synthetic 100 UL54
gaccuggaaucggacagcagcg

SEQ ID NO: 25 - RNA, 21 nt, Artificial Sequence
  Synthetic 616 UL6
gggaggacgacgagaccaagg

SEQ ID NO: 26 - RNA, 23 nt, Artificial Sequence
  Synthetic 178 RL2
gcggaggccgccgaggacgucag

SEQ ID NO: 27 - RNA, 24 nt, Artificial Sequence
  Synthetic 625 UL6
gaugucugucagguacguuugcag

SEQ ID NO: 28 - RNA, 24 nt, Artificial Sequence
  Synthetic 410 UL19
guugucgggcugcauaaacugcuc

SEQ ID NO: 29 - RNA, 22 nt, Artificial Sequence
  Synthetic 271 RL2
ggcucaggccgcgaaccaagaa

SEQ ID NO: 30 - RNA, 20 nt, Artificial Sequence
  Synthetic 398 UL19
ggggugcgcgggguccguca

SEQ ID NO: 31 - RNA, 23 nt, Artificial Sequence
  Synthetic 406 UL19
ggccugcagcagguaaaacaccg

SEQ ID NO: 32 - RNA, 21 nt, Artificial Sequence
  Synthetic 285 RL2
gcccauccuggacauggagac

SEQ ID NO: 33 - RNA, 23 nt, Artificial Sequence
  Synthetic 573 UL48
gucaacggagcgcucaccguccg

SEQ ID NO: 34 - RNA, 20 nt, Artificial Sequence
  Synthetic 614 UL6
gacgugcgcguguucccgga

SEQ ID NO: 35 - RNA, 22 nt, Artificial Sequence
  Synthetic 509 UL32
gacugcgccaggaacgaguagu

SEQ ID NO: 36 - RNA, 23 nt, Artificial Sequence
  Synthetic 613 UL6
gcagacgugcgcguguucccgga

SEQ ID NO: 37 - RNA, 20 nt, Artificial Sequence
  Synthetic 564 UL37
gcucuuggccgagaaccucc

SEQ ID NO: 38 - RNA, 24 nt, Artificial Sequence
  Synthetic 536 UL37
gcacgcacuaccuuucggcccugg

SEQ ID NO: 39 - RNA, 20 nt, Artificial Sequence
  Synthetic 508 UL32
gccaagguuccgaugagggc

SEQ ID NO: 40 - RNA, 24 nt, Artificial Sequence
  Synthetic 617 UL6
gauucggcaagggcggaaacagcc

SEQ ID NO: 41 - RNA, 24 nt, Artificial Sequence
  Synthetic 305 RL2
gcagguagcgcgugaggccgcccg

SEQ ID NO: 42 - RNA, 20 nt, Artificial Sequence
  Synthetic 416 UL19
guacggauuggcggcuucga

SEQ ID NO: 43 - RNA, 22 nt, Artificial Sequence
  Synthetic 440 UL22
ggacuucgaccggggucucgga

SEQ ID NO: 44 - RNA, 23 nt, Artificial Sequence
  Synthetic 426 UL19
guaauuguguguggguugcucga

SEQ ID NO: 45 - RNA, 23 nt, Artificial Sequence
  Synthetic 569 UL48
gggcgaggucgugaagcuggaau

SEQ ID NO: 46 - RNA, 23 nt, Artificial Sequence
  Synthetic 584 UL48
guaccaugcucgauaccuggaac

SEQ ID NO: 47 - RNA, 23 nt, Artificial Sequence
  Synthetic 494 UL29
guccaccucggugagauggaggg

SEQ ID NO: 48 - RNA, 24 nt, Artificial Sequence
  Synthetic 615 UL6
ggcagacgugcgcguguucccgga

SEQ ID NO: 49 - RNA, 22 nt, Artificial Sequence
  Synthetic 417 UL19
gccgcgucaacguucaucagcg

SEQ ID NO: 50 - RNA, 22 nt, Artificial Sequence
  Synthetic 430 UL19
gcuccagaaagcgcacgagcga

SEQ ID NO: 51 - RNA, 21 nt, Artificial Sequence
  Synthetic 337 RS1
guugcgcgcggcgcccgagau

SEQ ID NO: 52 - RNA, 24 nt, Artificial Sequence
  Synthetic 449 UL22
gggcggccaacccggcaagcgcgc

SEQ ID NO: 53 - RNA, 22 nt, Artificial Sequence
  Synthetic 391 UL19
gcgcugaccuacgcgcucaugg

SEQ ID NO: 54 - RNA, 24 nt, Artificial Sequence
  Synthetic 572 UL48
gcuggaauacgaguccaacuucgc

SEQ ID NO: 55 - RNA, 24 nt, Artificial Sequence
  Synthetic 369 UL15
gccgcgccacggccgagaagagcg

SEQ ID NO: 56 - RNA, 21 nt, Artificial Sequence
  Synthetic 368 UL1
gcccuacacagcggcgggugc

SEQ ID NO: 57 - RNA, 23 nt, Artificial Sequence
  Synthetic 525 UL37
gaagcccgggucggcgaggacgu

SEQ ID NO: 58 - RNA, 20 nt, Artificial Sequence
  Synthetic 452 UL22
ggccaacccggcaagcgcgc

SEQ ID NO: 59 - RNA, 20 nt, Artificial Sequence
  Synthetic 401 UL19
gcgggcaggcgcaagccgag

SEQ ID NO: 60 - RNA, 21 nt, Artificial Sequence
  Synthetic 313 RL2
ggaugcagaagcggugcaugc

SEQ ID NO: 61 - RNA, 22 nt, Artificial Sequence
  Synthetic 460 UL22
gugcauggauaucacgagcccc

SEQ ID NO: 62 - RNA, 20 nt, Artificial Sequence
  Synthetic 527 UL37
gcccgggucggcgaggacgu

SEQ ID NO: 63 - RNA, 22 nt, Artificial Sequence
  Synthetic 312 RL2
gggaugcagaagcggugcaugc

SEQ ID NO: 64 - RNA, 20 nt, Artificial Sequence
  Synthetic 367 UL1
gccaagcgcguuucuguuuc

SEQ ID NO: 65 - RNA, 22 nt, Artificial Sequence
  Synthetic 388 UL15
gugcgccugcagaccgacccgg

SEQ ID NO: 66 - RNA, 22 nt, Artificial Sequence
  Synthetic 343 RS1
gcgaggccgcggggucgggcgu

SEQ ID NO: 67 - RNA, 23 nt, Artificial Sequence
  Synthetic 304 RL2
guaaggcgacagggcgaccacgc

SEQ ID NO: 68 - RNA, 20 nt, Artificial Sequence
  Synthetic 389 UL15
gcgccugcagaccgacccgg

SEQ ID NO: 69 - RNA, 24 nt, Artificial Sequence
  Synthetic 433 UL19
gguaugccgcggccauggugccga

SEQ ID NO: 70 - RNA, 22 nt, Artificial Sequence
  Synthetic 383 UL15
gcucggguguuggccgugaacg

SEQ ID NO: 71 - RNA, 24 nt, Artificial Sequence
  Synthetic 400 UL19
ggcccuggaccgccaucgcgacug

SEQ ID NO: 72 - RNA, 20 nt, Artificial Sequence
  Synthetic 455 UL22
guuccugucgcgggcauacg

SEQ ID NO: 73 - RNA, 24 nt, Artificial Sequence
  Synthetic 392 UL19
gcggacgugcugcacgacgggcug

SEQ ID NO: 74 - RNA, 22 nt, Artificial Sequence
  Synthetic 393 UL19
ggacgugcugcacgacgggcug

SEQ ID NO: 75 - RNA, 24 nt, Artificial Sequence
  Synthetic 297 RL2
gugguuacccgcggucucggggag

SEQ ID NO: 76 - RNA, 23 nt, Artificial Sequence
  Synthetic 287 RL2
guacgugguccugguggaccaga

SEQ ID NO: 77 - RNA, 22 nt, Artificial Sequence
  Synthetic 448 UL22
gccucuagcugcaggcgggccg

SEQ ID NO: 78 - RNA, 23 nt, Artificial Sequence
  Synthetic 273 RL2
gagcaggaggcggcggugcgucc

SEQ ID NO: 79 - RNA, 24 nt, Artificial Sequence
  Synthetic 344 RS1
gggcggcggcccgucgguggggcc

SEQ ID NO: 80 - RNA, 21 nt, Artificial Sequence
  Synthetic 652 UL9
gagcacacucguguccggaga

SEQ ID NO: 81 - RNA, 21 nt, Artificial Sequence
  Synthetic 639 UL9
guccguguaccagucccuggg

SEQ ID NO: 82 - RNA, 24 nt, Artificial Sequence
  Synthetic 386 UL15
gcacgugcguggcgauggcgacgg

SEQ ID NO: 83 - RNA, 24 nt, Artificial Sequence
  Synthetic 574 UL48
gaccuggagagcuggcgucaguug

SEQ ID NO: 84 - RNA, 21 nt, Artificial Sequence
  Synthetic 566 UL37
gcuuggccguggccagaaacg

SEQ ID NO: 85 - RNA, 22 nt, Artificial Sequence
  Synthetic 451 UL22
gcggccaacccggcaagcgcgc

SEQ ID NO: 86 - RNA, 24 nt, Artificial Sequence
  Synthetic 362 RS1
gggaccuggccuucgccggggccg

SEQ ID NO: 87 - RNA, 22 nt, Artificial Sequence
  Synthetic 288 RL2
guacgugguccugguggaccag

SEQ ID NO: 88 - RNA, 24 nt, Artificial Sequence
  Synthetic 408 UL19
gccguucaccacgcgccagguggc

SEQ ID NO: 89 - RNA, 24 nt, Artificial Sequence
  Synthetic 321 RS1
gucgccggcgcuggcgagcagccc

SEQ ID NO: 90 - RNA, 24 nt, Artificial Sequence
  Synthetic 399 UL19
gaucuccgggaccaccauguucug

SEQ ID NO: 91 - RNA, 20 nt, Artificial Sequence
  Synthetic 511 UL32
ggcgagcgucgcggugugcg

SEQ ID NO: 92 - RNA, 23 nt, Artificial Sequence
  Synthetic 624 UL6
gacgaaugcggcgaucgccccgc

SEQ ID NO: 93 - RNA, 24 nt, Artificial Sequence
  Synthetic 585 UL49.5
gcccggcgaggagggcgggcgcga

SEQ ID NO: 94 - RNA, 24 nt, Artificial Sequence
  Synthetic 289 RL2
guacgugguccugguggaccagac

SEQ ID NO: 95 - RNA, 21 nt, Artificial Sequence
  Synthetic 553 UL37
ggccuccgguucuuggcccug

SEQ ID NO: 96 - RNA, 23 nt, Artificial Sequence
  Synthetic 523 UL37
ggccagaacguaggggacgaagc

SEQ ID NO: 97 - RNA, 24 nt, Artificial Sequence
  Synthetic 640 UL9
gacaccauguccgguccguaguuc

SEQ ID NO: 98 - RNA, 24 nt, Artificial Sequence
  Synthetic 552 UL37
gagggccuccgguucuuggcccug

SEQ ID NO: 99 - RNA, 21 nt, Artificial Sequence
  Synthetic 577 UL48
guacgcggcccauaggaucuc

SEQ ID NO: 100 - RNA, 24 nt, Artificial Sequence
  Synthetic 526 UL37
gcccgggucggcgaggacguuggg

SEQ ID NO: 101 - RNA, 24 nt, Artificial Sequence
  Synthetic 529 UL37
gugaaugcccucgagggcggggaa

SEQ ID NO: 102 - RNA, 20 nt, Artificial Sequence
  Synthetic 623 UL6
gaaugcggcgaucgccccgc

SEQ ID NO: 103 - RNA, 23 nt, Artificial Sequence
  Synthetic 342 RS1
ggcgaggccgcggggucgggcgu

SEQ ID NO: 104 - RNA, 23 nt, Artificial Sequence
  Synthetic 496 UL29
gaccagauacaggagggcgcggu

SEQ ID NO: 105 - RNA, 21 nt, Artificial Sequence
  Synthetic 465 UL22
gagaaauacacguaccuaccu

SEQ ID NO: 106 - RNA, 24 nt, Artificial Sequence
  Synthetic 513 UL33
ggucugggaccgcagcgcgcaguc

SEQ ID NO: 107 - RNA, 21 nt, Artificial Sequence
  Synthetic 657 UL9
guggaucgcuucccgcagcca

SEQ ID NO: 108 - RNA, 20 nt, Artificial Sequence
  Synthetic 528 UL37
gggucggcgaggacguuggg

SEQ ID NO: 109 - RNA, 24 nt, Artificial Sequence
  Synthetic 565 UL37
gcagcuuggccguggccagaaacg

SEQ ID NO: 110 - RNA, 23 nt, Artificial Sequence
  Synthetic 327 RS1
gucaaacaugagggccggucgcc

SEQ ID NO: 111 - RNA, 21 nt, Artificial Sequence
  Synthetic 601 UL6
gguauaccccagcugcccgug

SEQ ID NO: 112 - RNA, 24 nt, Artificial Sequence
  Synthetic 384 UL15
gcaucccggggcguuucgcggcgu

SEQ ID NO: 113 - RNA, 23 nt, Artificial Sequence
  Synthetic 537 UL37
gagggcgugaacccagcgucgau

SEQ ID NO: 114 - RNA, 21 nt, Artificial Sequence
  Synthetic 320 RL2
guccggcaggucucggucgca

SEQ ID NO: 115 - RNA, 24 nt, Artificial Sequence
  Synthetic 592 UL54
ggacgccgugggcgucgcaggggu

SEQ ID NO: 116 - RNA, 21 nt, Artificial Sequence
  Synthetic 456 UL22
gggcguaguucgugcccuccu

SEQ ID NO: 117 - RNA, 23 nt, Artificial Sequence
  Synthetic 512 UL33
gucugggaccgcagcgcgcaguc

SEQ ID NO: 118 - RNA, 21 nt, Artificial Sequence
  Synthetic 366 UL1
ggccaagcgcguuucuguuuc

SEQ ID NO: 119 - RNA, 24 nt, Artificial Sequence
  Synthetic 474 UL29
guuggagggccguccagaaccacu

SEQ ID NO: 120 - RNA, 23 nt, Artificial Sequence
  Synthetic 576 UL48
gcguacgcggcccauaggaucuc

SEQ ID NO: 121 - RNA, 24 nt, Artificial Sequence
  Synthetic 319 RL2
gcuguccggcaggucucggucgca

SEQ ID NO: 122 - RNA, 21 nt, Artificial Sequence
  Synthetic 583 UL48
gccgugggcgcgaaugucgau

SEQ ID NO: 123 - RNA, 21 nt, Artificial Sequence
  Synthetic 550 UL37
gagggcccgugugaggggagu

SEQ ID NO: 124 - RNA, 23 nt, Artificial Sequence
  Synthetic 590 UL54
gcggcggacgccgugggcgucgc

SEQ ID NO: 125 - RNA, 24 nt, Artificial Sequence
  Synthetic 493 UL29
guccaccuguggguuggccgccac

SEQ ID NO: 126 - RNA, 24 nt, Artificial Sequence
  Synthetic 522 UL37
gggccagaacguaggggacgaagc

SEQ ID NO: 127 - RNA, 21 nt, Artificial Sequence
  Synthetic 600 UL54
ggggcgcagcggcagguugug

SEQ ID NO: 128 - RNA, 24 nt, Artificial Sequence
  Synthetic 260 RL2
gccuccgaggcggacagcacggac

SEQ ID NO: 129 - RNA, 22 nt, Artificial Sequence
  Synthetic 374 UL15
gcgguggcgcgacugcgguuua

SEQ ID NO: 130 - RNA, 20 nt, Artificial Sequence
  Synthetic 457 UL22
ggcguaguucgugcccuccu

SEQ ID NO: 131 - RNA, 22 nt, Artificial Sequence
  Synthetic 431 UL19
gacgucgaguucgacgcccugc

SEQ ID NO: 132 - RNA, 24 nt, Artificial Sequence
  Synthetic 268 RL2
gcgacgugugcgccgugugcacgg

SEQ ID NO: 133 - RNA, 20 nt, Artificial Sequence
  Synthetic 641 UL9
ggucggcgaguacgccaugc

SEQ ID NO: 134 - RNA, 23 nt, Artificial Sequence
  Synthetic 316 RL2
guccgugcuguccgccucggagg

SEQ ID NO: 135 - RNA, 24 nt, Artificial Sequence
  Synthetic 340 RS1
gagccaccccauggccuccgcguc

SEQ ID NO: 136 - RNA, 23 nt, Artificial Sequence
  Synthetic 593 UL54
gacgccgugggcgucgcaggggu

SEQ ID NO: 137 - RNA, 24 nt, Artificial Sequence
  Synthetic 582 UL48
gucgccgugggcgcgaaugucgau

SEQ ID NO: 138 - RNA, 22 nt, Artificial Sequence
  Synthetic 265 RL2
gggcgacgugugcgccgugugc

SEQ ID NO: 139 - RNA, 24 nt, Artificial Sequence
  Synthetic 372 UL15
gggcgguggcgcgacugcgguuua

SEQ ID NO: 140 - RNA, 24 nt, Artificial Sequence
  Synthetic 447 UL22
gagccucuagcugcaggcgggccg

SEQ ID NO: 141 - RNA, 21 nt, Artificial Sequence
  Synthetic 409 UL19
guucaccacgcgccagguggc

SEQ ID NO: 142 - RNA, 23 nt, Artificial Sequence
  Synthetic 649 UL9
gacgacgagcacacucguguccg

SEQ ID NO: 143 - RNA, 24 nt, Artificial Sequence
  Synthetic 510 UL32
gagccucguuaagagccaggaggc

SEQ ID NO: 144 - RNA, 23 nt, Artificial Sequence
  Synthetic 598 UL54
ggccuuggcggucgaugcggccc

SEQ ID NO: 145 - RNA, 23 nt, Artificial Sequence
  Synthetic 462 UL22
gcuggugcgcgcgcgcuacgggc

SEQ ID NO: 146 - RNA, 20 nt, Artificial Sequence
  Synthetic 659 UL9
gacugugguccgcgcgccaa

SEQ ID NO: 147 - RNA, 24 nt, Artificial Sequence
  Synthetic 635 UL9
gcaguuuagggucagcagcaugug

SEQ ID NO: 148 - RNA, 20 nt, Artificial Sequence
  Synthetic 371 UL15
gggcgaccacacggccaagc

SEQ ID NO: 149 - RNA, 21 nt, Artificial Sequence
  Synthetic 414 UL19
gggccgccacguacgccccgu

SEQ ID NO: 150 - RNA, 20 nt, Artificial Sequence
  Synthetic 446 UL22
gcuaucagcuggccuucgug

SEQ ID NO: 151 - RNA, 22 nt, Artificial Sequence
  Synthetic 545 UL37
gcagcagacguggacguugauc

SEQ ID NO: 152 - RNA, 23 nt, Artificial Sequence
  Synthetic 373 UL15
ggcgguggcgcgacugcgguuua

SEQ ID NO: 153 - RNA, 20 nt, Artificial Sequence
  Synthetic 415 UL19
ggccgccacguacgccccgu

SEQ ID NO: 154 - RNA, 21 nt, Artificial Sequence
  Synthetic 581 UL48
gaaggccacgucgccgugggc

SEQ ID NO: 155 - RNA, 21 nt, Artificial Sequence
  Synthetic 419 UL19
gcgcggcaaaccguuccauga

SEQ ID NO: 156 - RNA, 22 nt, Artificial Sequence
  Synthetic 580 UL48
ggaaggccacgucgccgugggc

SEQ ID NO: 157 - RNA, 24 nt, Artificial Sequence
  Synthetic 620 UL6
gguccugggcguacgaagggaugu

SEQ ID NO: 158 - RNA, 24 nt, Artificial Sequence
  Synthetic 575 UL48
ggcguacgcggcccauaggaucuc

SEQ ID NO: 159 - RNA, 22 nt, Artificial Sequence
  Synthetic 364 RS1
gaccuggccuucgccggggccg

SEQ ID NO: 160 - RNA, 21 nt, Artificial Sequence
  Synthetic 633 UL9
gaucuacgggagcugcggugc

SEQ ID NO: 161 - RNA, 21 nt, Artificial Sequence
  Synthetic 521 UL37
gccccgggcggccgguucgag

SEQ ID NO: 162 - RNA, 22 nt, Artificial Sequence
  Synthetic 631 UL9
gccugggccggcagcgacgccu

SEQ ID NO: 163 - RNA, 20 nt, Artificial Sequence
  Synthetic 377 UL15
guaacgccuacuauagcguu

SEQ ID NO: 164 - RNA, 22 nt, Artificial Sequence
  Synthetic 654 UL9
ggaagcgauccacucuccggac

SEQ ID NO: 165 - RNA, 22 nt, Artificial Sequence
  Synthetic 298 RL2
gguuacccgcggucucggggag

SEQ ID NO: 166 - RNA, 20 nt, Artificial Sequence
  Synthetic 290 RL2
gugguccugguggaccagac

SEQ ID NO: 167 - RNA, 21 nt, Artificial Sequence
  Synthetic 293 RL2
gcggucucggcacccgugguc

SEQ ID NO: 168 - RNA, 22 nt, Artificial Sequence
  Synthetic 646 UL9
gcggucguucauaauguaauug

SEQ ID NO: 169 - RNA, 22 nt, Artificial Sequence
  Synthetic 438 UL22
ggccaggacuucgaccgggguc

SEQ ID NO: 170 - RNA, 24 nt, Artificial Sequence
  Synthetic 380 UL15
gucgacgaggccaacuuuauucgc

SEQ ID NO: 171 - RNA, 23 nt, Artificial Sequence
  Synthetic 397 UL19
gaccguguuggccaccagauugg

SEQ ID NO: 172 - RNA, 22 nt, Artificial Sequence
  Synthetic 318 RL2
gcuguccggcaggucucggucg

SEQ ID NO: 173 - RNA, 23 nt, Artificial Sequence
  Synthetic 382 UL15
ggcucggguguuggccgugaacg

SEQ ID NO: 174 - RNA, 21 nt, Artificial Sequence
  Synthetic 591 UL54
ggcggacgccgugggcgucgc

SEQ ID NO: 175 - RNA, 22 nt, Artificial Sequence
  Synthetic 621 UL6
gcuccagguccugggcguacga

SEQ ID NO: 176 - RNA, 21 nt, Artificial Sequence
  Synthetic 394 UL19
gacgugcugcacgacgggcug

SEQ ID NO: 177 - RNA, 24 nt, Artificial Sequence
  Synthetic 286 RL2
gcguacgugguccugguggaccag

SEQ ID NO: 178 - RNA, 23 nt, Artificial Sequence
  Synthetic 506 UL32
gaggccaagguuccgaugagggc

SEQ ID NO: 179 - RNA, 22 nt, Artificial Sequence
  Synthetic 540 UL37
gugaucaacacccugaucgacg

SEQ ID NO: 180 - RNA, 21 nt, Artificial Sequence
  Synthetic 567 UL37
gguccguccgagggcggaccg

SEQ ID NO: 181 - RNA, 23 nt, Artificial Sequence
  Synthetic 428 UL19
gucgcucgugcgcuuucuggagc

SEQ ID NO: 182 - RNA, 21 nt, Artificial Sequence
  Synthetic 507 UL32
ggccaagguuccgaugagggc

SEQ ID NO: 183 - RNA, 23 nt, Artificial Sequence
  Synthetic 317 RL2
gcuguccggcaggucucggucgc

SEQ ID NO: 184 - RNA, 24 nt, Artificial Sequence
  Synthetic 516 UL33
gacguagcgcgcgucuagacucuc

SEQ ID NO: 185 - RNA, 23 nt, Artificial Sequence
  Synthetic 464 UL22
gggagaaauacacguaccuaccu

SEQ ID NO: 186 - RNA, 21 nt, Artificial Sequence
  Synthetic 439 UL22
gccaggacuucgaccgggguc

SEQ ID NO: 187 - RNA, 21 nt, Artificial Sequence
  Synthetic 381 UL15
gacgaggccaacuuuauucgc

SEQ ID NO: 188 - RNA, 23 nt, Artificial Sequence
  Synthetic 562 UL37
gacgugucggagccggugaccgc

SEQ ID NO: 189 - RNA, 23 nt, Artificial Sequence
  Synthetic 425 UL19
gccuggacguugcgagcgagcug

SEQ ID NO: 190 - RNA, 21 nt, Artificial Sequence
  Synthetic 655 UL9
gaagcgauccacucuccggac

SEQ ID NO: 191 - RNA, 20 nt, Artificial Sequence
  Synthetic 346 RS1
ggcggcccgucgguggggcc

SEQ ID NO: 192 - RNA, 24 nt, Artificial Sequence
  Synthetic 505 UL32
ggaggccaagguuccgaugagggc

SEQ ID NO: 193 - RNA, 20 nt, Artificial Sequence
  Synthetic 375 UL15
gguggcgcgacugcgguuua

SEQ ID NO: 194 - RNA, 23 nt, Artificial Sequence
  Synthetic 656 UL9
gaguggaucgcuucccgcagcca

SEQ ID NO: 195 - RNA, 20 nt, Artificial Sequence
  Synthetic 453 UL22
ggcccacucgcgcgcgcuug

SEQ ID NO: 196 - RNA, 21 nt, Artificial Sequence
  Synthetic 283 RL2
gugcccggaagacgcgccacg

SEQ ID NO: 197 - RNA, 24 nt, Artificial Sequence
  Synthetic 661 UL9
guacacgucgagcguaucgcuagc

SEQ ID NO: 198 - RNA, 24 nt, Artificial Sequence
  Synthetic 622 UL6
gcgcuccagguccugggcguacga

SEQ ID NO: 199 - RNA, 23 nt, Artificial Sequence
  Synthetic 638 UL9
guguccguguaccagucccuggg

SEQ ID NO: 200 - RNA, 23 nt, Artificial Sequence
  Synthetic 387 UL15
ggugcgccugcagaccgacccgg

SEQ ID NO: 201 - RNA, 22 nt, Artificial Sequence
  Synthetic 471 UL22
ggcgguguaucacgcggcccgc

SEQ ID NO: 202 - RNA, 23 nt, Artificial Sequence
  Synthetic 533 UL37
gcucuuugggugcauugcccucg

SEQ ID NO: 203 - RNA, 20 nt, Artificial Sequence
  Synthetic 549 UL37
guaaagagggcccgugugag

SEQ ID NO: 204 - RNA, 20 nt, Artificial Sequence
  Synthetic 519 UL33
ggaaccgguuaaugaccgcc

SEQ ID NO: 205 - RNA, 24 nt, Artificial Sequence
  Synthetic 534 UL37
gacucggcgaaguccagcagcucu

SEQ ID NO: 206 - RNA, 24 nt, Artificial Sequence
  Synthetic 637 UL9
gguguccguguaccagucccuggg

SEQ ID NO: 207 - RNA, 23 nt, Artificial Sequence
  Synthetic 454 UL22
gccuaacggggcugcucgcgacg

SEQ ID NO: 208 - RNA, 24 nt, Artificial Sequence
  Synthetic 488 UL29
ggccaggugcucgccggcaaauuc

SEQ ID NO: 209 - RNA, 24 nt, Artificial Sequence
  Synthetic 586 UL54
gggcggaccgccgggcgagcggcg

SEQ ID NO: 210 - RNA, 22 nt, Artificial Sequence
  Synthetic 658 UL9
gugacugugguccgcgcgccaa

SEQ ID NO: 211 - RNA, 21 nt, Artificial Sequence
  Synthetic 445 UL22
ggcuaucagcuggccuucgug

SEQ ID NO: 212 - RNA, 23 nt, Artificial Sequence
  Synthetic 363 RS1
ggaccuggccuucgccggggccg

SEQ ID NO: 213 - RNA, 24 nt, Artificial Sequence
  Synthetic 429 UL19
gagcuccagaaagcgcacgagcga

SEQ ID NO: 214 - RNA, 20 nt, Artificial Sequence
  Synthetic 294 RL2
ggggcgucgacccgggacga

SEQ ID NO: 215 - RNA, 24 nt, Artificial Sequence
  Synthetic 458 UL22
gguccccgcgggccagacgcuaga

SEQ ID NO: 216 - RNA, 21 nt, Artificial Sequence
  Synthetic 306 RL2
gguagcgcgugaggccgcccg

SEQ ID NO: 217 - RNA, 22 nt, Artificial Sequence
  Synthetic 352 RS1
gccuucuacgcgcgcuaucgcg

SEQ ID NO: 218 - RNA, 22 nt, Artificial Sequence
  Synthetic 475 UL29
gcagaaagcccagcgguccgag

SEQ ID NO: 219 - RNA, 21 nt, Artificial Sequence
  Synthetic 647 UL9
gugcucgucgucuccugucgu

SEQ ID NO: 220 - RNA, 23 nt, Artificial Sequence
  Synthetic 357 RS1
guggcgcuggaccaggccugcuu

SEQ ID NO: 221 - RNA, 21 nt, Artificial Sequence
  Synthetic 421 UL19
gcuucgcuuuagcucggcgac

SEQ ID NO: 222 - RNA, 24 nt, Artificial Sequence
  Synthetic 520 UL37
gccgccccgggcggccgguucgag

SEQ ID NO: 223 - RNA, 20 nt, Artificial Sequence
  Synthetic 599 UL54
gggcgcagcggcagguugug

SEQ ID NO: 224 - RNA, 23 nt, Artificial Sequence
  Synthetic 365 UL1
gucccggauuggacacggucuug

SEQ ID NO: 225 - RNA, 24 nt, Artificial Sequence
  Synthetic 463 UL22
ggggagaaauacacguaccuaccu

SEQ ID NO: 226 - RNA, 24 nt, Artificial Sequence
  Synthetic 308 RL2
gcaccgacggccccgcccgaggag

SEQ ID NO: 227 - RNA, 20 nt, Artificial Sequence
  Synthetic 487 UL29
gguccacguacugcaggguc

SEQ ID NO: 228 - RNA, 23 nt, Artificial Sequence
  Synthetic 619 UL6
guccugggcguacgaagggaugu

SEQ ID NO: 229 - RNA, 24 nt, Artificial Sequence
  Synthetic 326 RS1
ggucaaacaugagggccggucgcc

SEQ ID NO: 230 - RNA, 23 nt, Artificial Sequence
  Synthetic 282 RL2
gugugcccggaagacgcgccacg

SEQ ID NO: 231 - RNA, 22 nt, Artificial Sequence
  Synthetic 347 RS1
gaccucgacgacgcccggcggg

SEQ ID NO: 232 - RNA, 23 nt, Artificial Sequence
  Synthetic 333 RS1
ggagcugcuguugcgcgcggcgc

SEQ ID NO: 233 - RNA, 20 nt, Artificial Sequence
  Synthetic 307 RL2
guagcgcgugaggccgcccg

SEQ ID NO: 234 - RNA, 23 nt, Artificial Sequence
  Synthetic 630 UL9
ggccugggccggcagcgacgccu

SEQ ID NO: 235 - RNA, 20 nt, Artificial Sequence
  Synthetic 270 RL2
gauagugggcgugacgccca

SEQ ID NO: 236 - RNA, 21 nt, Artificial Sequence
  Synthetic 636 UL9
guuuagggucagcagcaugug

SEQ ID NO: 237 - RNA, 20 nt, Artificial Sequence
  Synthetic 423 UL19
gacaacggccgccuggccac

SEQ ID NO: 238 - RNA, 21 nt, Artificial Sequence
  Synthetic 578 UL48
guaguagagcccgaggccguc

SEQ ID NO: 239 - RNA, 21 nt, Artificial Sequence
  Synthetic 302 RL2
gcagggugcggcggcuccagc

SEQ ID NO: 240 - RNA, 21 nt, Artificial Sequence
  Synthetic 378 UL15
gcggggcugguucgguucggc

SEQ ID NO: 241 - RNA, 22 nt, Artificial Sequence
  Synthetic 436 UL19
gccgcggcauaccgguaucccg

SEQ ID NO: 242 - RNA, 21 nt, Artificial Sequence
  Synthetic 379 UL15
gagcagguuaaaguccuggcc

SEQ ID NO: 243 - RNA, 20 nt, Artificial Sequence
  Synthetic 542 UL37
gaucaacacccugaucgacg

SEQ ID NO: 244 - RNA, 21 nt, Artificial Sequence
  Synthetic 266 RL2
ggcgacgugugcgccgugugc

SEQ ID NO: 245 - RNA, 22 nt, Artificial Sequence
  Synthetic 558 UL37
guccgcguccgccagugcgccg

SEQ ID NO: 246 - RNA, 23 nt, Artificial Sequence
  Synthetic 395 UL19
ggauuccggcgugcaacgccccg

SEQ ID NO: 247 - RNA, 20 nt, Artificial Sequence
  Synthetic 612 UL6
gucguaguccgccacgcgcg

SEQ ID NO: 248 - RNA, 24 nt, Artificial Sequence
  Synthetic 498 UL32
gagucgccggauacgugggcguac

SEQ ID NO: 249 - RNA, 23 nt, Artificial Sequence
  Synthetic 653 UL9
gggaagcgauccacucuccggac

SEQ ID NO: 250 - RNA, 23 nt, Artificial Sequence
  Synthetic 434 UL19
guaugccgcggccauggugccga

SEQ ID NO: 251 - RNA, 23 nt, Artificial Sequence
  Synthetic 437 UL22
gggccaggacuucgaccgggguc

SEQ ID NO: 252 - RNA, 24 nt, Artificial Sequence
  Synthetic 336 RS1
gcuguugcgcgcggcgcccgagau

SEQ ID NO: 253 - RNA, 22 nt, Artificial Sequence
  Synthetic 643 UL9
guggucggcgaguacgccaugc

SEQ ID NO: 254 - RNA, 20 nt, Artificial Sequence
  Synthetic 328 RS1
gagggccggucgccagggga

SEQ ID NO: 255 - RNA, 23 nt, Artificial Sequence
  Synthetic 435 UL19
ggccgcggcauaccgguaucccg

SEQ ID NO: 256 - RNA, 21 nt, Artificial Sequence
  Synthetic 422 UL19
ggacaacggccgccuggccac

SEQ ID NO: 257 - RNA, 21 nt, Artificial Sequence
  Synthetic 299 RL2
guuacccgcggucucggggag

SEQ ID NO: 258 - RNA, 21 nt, Artificial Sequence
  Synthetic 588 UL54
gucggacgaggcgucugggug

SEQ ID NO: 259 - RNA, 22 nt, Artificial Sequence
  Synthetic 341 RS1
gccaccccauggccuccgcguc

SEQ ID NO: 260 - RNA, 21 nt, Artificial Sequence
  Synthetic 322 RS1
gccggcgcuggcgagcagccc

SEQ ID NO: 261 - RNA, 23 nt, Artificial Sequence
  Synthetic 470 UL22
gggcgguguaucacgcggcccgc

SEQ ID NO: 262 - RNA, 24 nt, Artificial Sequence
  Synthetic 660 UL9
gcgugacugugguccgcgcgccaa

SEQ ID NO: 263 - RNA, 23 nt, Artificial Sequence
  Synthetic 418 UL19
gugcgcggcaaaccguuccauga

SEQ ID NO: 264 - RNA, 22 nt, Artificial Sequence
  Synthetic 269 RL2
gacgugugcgccgugugcacgg

SEQ ID NO: 265 - RNA, 20 nt, Artificial Sequence
  Synthetic 335 RS1
gcugcuguugcgcgcggcgc

SEQ ID NO: 266 - RNA, 21 nt, Artificial Sequence
  Synthetic 370 UL15
ggggcgaccacacggccaagc

SEQ ID NO: 267 - RNA, 21 nt, Artificial Sequence
  Synthetic 315 RL2
gaugcagaagcggugcaugca

SEQ ID NO: 268 - RNA, 23 nt, Artificial Sequence
  Synthetic 632 UL9
gcgaucuacgggagcugcggugc

SEQ ID NO: 269 - RNA, 23 nt, Artificial Sequence
  Synthetic 459 UL22
guccccgcgggccagacgcuaga

SEQ ID NO: 270 - RNA, 20 nt, Artificial Sequence
  Synthetic 325 RS1
ggcgaucgaggccagggccc

SEQ ID NO: 271 - RNA, 21 nt, Artificial Sequence
  Synthetic 309 RL2
gaggaggacgcggccccggcg

SEQ ID NO: 272 - RNA, 21 nt, Artificial Sequence
  Synthetic 485 UL29
gaccgccugguccacguacug

SEQ ID NO: 273 - RNA, 23 nt, Artificial Sequence
  Synthetic 259 RL2
gaugucuggguguuucccugcga

SEQ ID NO: 274 - RNA, 21 nt, Artificial Sequence
  Synthetic 543 UL37
ggguguugaucaccacggagg

SEQ ID NO: 275 - RNA, 20 nt, Artificial Sequence
  Synthetic 532 UL37
gcgccgucucgcggagaagg

SEQ ID NO: 276 - RNA, 24 nt, Artificial Sequence
  Synthetic 517 UL33
gcgcaugacgcggccgucugguuc

SEQ ID NO: 277 - RNA, 23 nt, Artificial Sequence
  Synthetic 645 UL9
ggcggucguucauaauguaauug

SEQ ID NO: 278 - RNA, 22 nt, Artificial Sequence
  Synthetic 262 RL2
gacagcacggacacggaacugu

SEQ ID NO: 279 - RNA, 20 nt, Artificial Sequence
  Synthetic 563 UL37
gugucggagccggugaccgc

SEQ ID NO: 280 - RNA, 20 nt, Artificial Sequence
  Synthetic 332 RS1
gugagcucggccacggcgcg

SEQ ID NO: 281 - RNA, 21 nt, Artificial Sequence
  Synthetic 611 UL6
ggucguaguccgccacgcgcg

SEQ ID NO: 282 - RNA, 24 nt, Artificial Sequence
  Synthetic 476 UL29
gacgcgcagcuccuugggaaaccc

SEQ ID NO: 283 - RNA, 23 nt, Artificial Sequence
  Synthetic 648 UL9
gugugcucgucgucuccugucgu

SEQ ID NO: 284 - RNA, 24 nt, Artificial Sequence
  Synthetic 629 UL9
gcccaggccgccgagacggaggag

SEQ ID NO: 285 - RNA, 23 nt, Artificial Sequence
  Synthetic 610 UL6
gcggucguaguccgccacgcgcg

SEQ ID NO: 286 - RNA, 21 nt, Artificial Sequence
  Synthetic 330 RS1
gucgccaggggacggggaaca

SEQ ID NO: 287 - RNA, 23 nt, Artificial Sequence
  Synthetic 477 UL29
gggcgacguugcccgcgugaccc

SEQ ID NO: 288 - RNA, 23 nt, Artificial Sequence
  Synthetic 642 UL9
gguggucggcgaguacgccaugc

SEQ ID NO: 289 - RNA, 23 nt, Artificial Sequence
  Synthetic 579 UL48
gggaaggccacgucgccgugggc

SEQ ID NO: 290 - RNA, 23 nt, Artificial Sequence
  Synthetic 500 UL32
ggucgccggggaccgcgagccgg

SEQ ID NO: 291 - RNA, 23 nt, Artificial Sequence
  Synthetic 261 RL2
ggacagcacggacacggaacugu

SEQ ID NO: 292 - RNA, 21 nt, Artificial Sequence
  Synthetic 324 RS1
gcaccaccacgcgcacguccu

SEQ ID NO: 293 - RNA, 21 nt, Artificial Sequence
  Synthetic 518 UL33
gccgugcacacacgcgguguc

SEQ ID NO: 294 - RNA, 21 nt, Artificial Sequence
  Synthetic 311 RL2
gguucgcggccugagccaggg

SEQ ID NO: 295 - RNA, 20 nt, Artificial Sequence
  Synthetic 539 UL37
ggcgugaacccagcgucgau

SEQ ID NO: 296 - RNA, 21 nt, Artificial Sequence
  Synthetic 538 UL37
gggcgugaacccagcgucgau

SEQ ID NO: 297 - RNA, 24 nt, Artificial Sequence
  Synthetic 420 UL19
gaagcuucgcuuuagcucggcgac

SEQ ID NO: 298 - RNA, 20 nt, Artificial Sequence
  Synthetic 292 RL2
gcgucggaguggaacagccu

SEQ ID NO: 299 - RNA, 23 nt, Artificial Sequence
  Synthetic 489 UL29
gccaggugcucgccggcaaauuc

SEQ ID NO: 300 - RNA, 20 nt, Artificial Sequence
  Synthetic 556 UL37
guccgccagugcgccgugga

SEQ ID NO: 301 - RNA, 22 nt, Artificial Sequence
  Synthetic 595 UL54
ggcccuuugacgccgagaccag

SEQ ID NO: 302 - RNA, 20 nt, Artificial Sequence
  Synthetic 473 UL22
guguugcugcguucccgggc

SEQ ID NO: 303 - RNA, 21 nt, Artificial Sequence
  Synthetic 515 UL33
guagcgcgcgucuagacucuc

SEQ ID NO: 304 - RNA, 24 nt, Artificial Sequence
  Synthetic 264 RL2
gagggcgacgugugcgccgugugc

SEQ ID NO: 305 - RNA, 22 nt, Artificial Sequence
  Synthetic 413 UL19
ggggccgccacguacgccccgu

SEQ ID NO: 306 - RNA, 24 nt, Artificial Sequence
  Synthetic 412 UL19
gcgccuggcccacgggcggguccg

SEQ ID NO: 307 - RNA, 21 nt, Artificial Sequence
  Synthetic 263 RL2
gggcugcuggggccgcagggc

SEQ ID NO: 308 - RNA, 22 nt, Artificial Sequence
  Synthetic 354 RS1
gcuaucgcgacggguacgucag

SEQ ID NO: 309 - RNA, 20 nt, Artificial Sequence
  Synthetic 587 UL54
ggaccgccgggcgagcggcg

SEQ ID NO: 310 - RNA, 24 nt, Artificial Sequence
  Synthetic 561 UL37
ggacgugucggagccggugaccgc

SEQ ID NO: 311 - RNA, 20 nt, Artificial Sequence
  Synthetic 568 UL37
guccguccgagggcggaccg

SEQ ID NO: 312 - RNA, 22 nt, Artificial Sequence
  Synthetic 546 UL37
gacgaacucaggaacggcacgu

SEQ ID NO: 313 - RNA, 23 nt, Artificial Sequence
  Synthetic 502 UL32
gcucgcgguccccggcgaccguc

SEQ ID NO: 314 - RNA, 24 nt, Artificial Sequence
  Synthetic 486 UL29
gccugguccacguacugcaggguc

SEQ ID NO: 315 - RNA, 24 nt, Artificial Sequence
  Synthetic 300 RL2
ggagcagggugcggcggcuccagc

SEQ ID NO: 316 - RNA, 23 nt, Artificial Sequence
  Synthetic 323 RS1
ggccagguccucgcccggcagcg

SEQ ID NO: 317 - RNA, 22 nt, Artificial Sequence
  Synthetic 627 UL9
gcccugcuuauggcccacaagc

SEQ ID NO: 318 - RNA, 24 nt, Artificial Sequence
  Synthetic 499 UL32
ggggaccgcgagccggaggagucg

SEQ ID NO: 319 - RNA, 20 nt, Artificial Sequence
  Synthetic 314 RL2
gaugcagaagcggugcaugc

SEQ ID NO: 320 - RNA, 24 nt, Artificial Sequence
  Synthetic 548 UL37
guguuugaucccgacgugccguuc

SEQ ID NO: 321 - RNA, 24 nt, Artificial Sequence
  Synthetic 530 UL37
gcccucgagggcggggaagcggau

SEQ ID NO: 322 - RNA, 21 nt, Artificial Sequence
  Synthetic 296 RL2
gcggaaguccagggcgcccac

SEQ ID NO: 323 - RNA, 23 nt, Artificial Sequence
  Synthetic 467 UL22
gugcgugauguccagcuccgucg

SEQ ID NO: 324 - RNA, 22 nt, Artificial Sequence
  Synthetic 490 UL29
gcgccccucgucggcggaaccc

SEQ ID NO: 325 - RNA, 24 nt, Artificial Sequence
  Synthetic 501 UL32
ggcucgcgguccccggcgaccguc

SEQ ID NO: 326 - RNA, 24 nt, Artificial Sequence
  Synthetic 331 RS1
guccgugagcucggccacggcgcg

SEQ ID NO: 327 - RNA, 23 nt, Artificial Sequence
  Synthetic 483 UL29
gggaccgccugguccacguacug

SEQ ID NO: 328 - RNA, 20 nt, Artificial Sequence
  Synthetic 560 UL37
gcuccgacacgucccaugcg

SEQ ID NO: 329 - RNA, 22 nt, Artificial Sequence
  Synthetic 444 UL22
gggcuaucagcuggccuucgug

SEQ ID NO: 330 - RNA, 24 nt, Artificial Sequence
  Synthetic 628 UL9
gauccgccgaaauauggcgcugcu

SEQ ID NO: 331 - RNA, 24 nt, Artificial Sequence
  Synthetic 482 UL29
ggggaccgccugguccacguacug

SEQ ID NO: 332 - RNA, 24 nt, Artificial Sequence
  Synthetic 351 RS1
gcgccuucuacgcgcgcuaucgcg

SEQ ID NO: 333 - RNA, 22 nt, Artificial Sequence
  Synthetic 597 UL54
gccuuggcggucgaugcggccc

SEQ ID NO: 334 - RNA, 24 nt, Artificial Sequence
  Synthetic 358 RS1
gggcccgcugcgccgcauggcggc

SEQ ID NO: 335 - RNA, 23 nt, Artificial Sequence
  Synthetic 355 RS1
guacaccccggacgcggaggcca

SEQ ID NO: 336 - RNA, 22 nt, Artificial Sequence
  Synthetic 547 UL37
guuugaucccgacgugccguuc

SEQ ID NO: 337 - RNA, 20 nt, Artificial Sequence
  Synthetic 544 UL37
gguguugaucaccacggagg

SEQ ID NO: 338 - RNA, 23 nt, Artificial Sequence
  Synthetic 557 UL37
gguccgcguccgccagugcgccg

SEQ ID NO: 339 - RNA, 24 nt, Artificial Sequence
  Synthetic 258 RL2
ggaugucuggguguuucccugcga

SEQ ID NO: 340 - RNA, 20 nt, Artificial Sequence
  Synthetic 461 UL22
gcauggauaucacgagcccc

SEQ ID NO: 341 - RNA, 24 nt, Artificial Sequence
  Synthetic 424 UL19
ggccuggacguugcgagcgagcug

SEQ ID NO: 342 - RNA, 24 nt, Artificial Sequence
  Synthetic 651 UL9
gacgagcacacucguguccggaga

SEQ ID NO: 343 - RNA, 22 nt, Artificial Sequence
  Synthetic 589 UL54
gccgcuccgucggacgaggcgu

SEQ ID NO: 344 - RNA, 22 nt, Artificial Sequence
  Synthetic 376 UL15
gcguaacgccuacuauagcguu

SEQ ID NO: 345 - RNA, 23 nt, Artificial Sequence
  Synthetic 348 RS1
ggcgucaucgaccucgucggacu

SEQ ID NO: 346 - RNA, 24 nt, Artificial Sequence
  Synthetic 284 RL2
gccugcccauccuggacauggaga

SEQ ID NO: 347 - RNA, 21 nt, Artificial Sequence
  Synthetic 559 UL37
ggcuccgacacgucccaugcg

SEQ ID NO: 348 - RNA, 20 nt, Artificial Sequence
  Synthetic 650 UL9
gacgagcacacucguguccg

SEQ ID NO: 349 - RNA, 20 nt, Artificial Sequence
  Synthetic 267 RL2
gcgacgugugcgccgugugc

SEQ ID NO: 350 - RNA, 24 nt, Artificial Sequence
  Synthetic 604 UL6
ggcggauaucgccgggguggccga

SEQ ID NO: 351 - RNA, 24 nt, Artificial Sequence
  Synthetic 554 UL37
ggcggggaagagcaggcuguacgc

SEQ ID NO: 352 - RNA, 20 nt, Artificial Sequence
  Synthetic 361 RS1
ggcgugcugcugcuguccac

SEQ ID NO: 353 - RNA, 23 nt, Artificial Sequence
  Synthetic 541 UL37
ggugaucaacacccugaucgacg

SEQ ID NO: 354 - RNA, 21 nt, Artificial Sequence
  Synthetic 634 UL9
gaagcgcacgcguaugcaguu

SEQ ID NO: 355 - RNA, 21 nt, Artificial Sequence
  Synthetic 402 UL19
ggcgggcaggcgcaagccgag

SEQ ID NO: 356 - RNA, 22 nt, Artificial Sequence
  Synthetic 349 RS1
gcgucaucgaccucgucggacu

SEQ ID NO: 357 - RNA, 22 nt, Artificial Sequence
  Synthetic 310 RL2
guucuugguucgcggccugagc

SEQ ID NO: 358 - RNA, 23 nt, Artificial Sequence
  Synthetic 605 UL6
gcggauaucgccgggguggccga

SEQ ID NO: 359 - RNA, 20 nt, Artificial Sequence
  Synthetic 491 UL29
gccccucgucggcggaaccc

SEQ ID NO: 360 - RNA, 22 nt, Artificial Sequence
  Synthetic 334 RS1
gagcugcuguugcgcgcggcgc

SEQ ID NO: 361 - RNA, 23 nt, Artificial Sequence
  Synthetic 291 RL2
gccgcacccugcuccccgagacc

SEQ ID NO: 362 - RNA, 21 nt, Artificial Sequence
  Synthetic 478 UL29
gcgacguugcccgcgugaccc

SEQ ID NO: 363 - RNA, 24 nt, Artificial Sequence
  Synthetic 618 UL6
gacccgcggcuguuuccgcccuug

SEQ ID NO: 364 - RNA, 20 nt, Artificial Sequence
  Synthetic 427 UL19
gcucgugcgcuuucuggagc

SEQ ID NO: 365 - RNA, 24 nt, Artificial Sequence
  Synthetic 644 UL9
gggcggucguucauaauguaauug

SEQ ID NO: 366 - RNA, 24 nt, Artificial Sequence
  Synthetic 356 RS1
gcuccagaacccgcgcgugguccc

SEQ ID NO: 367 - RNA, 22 nt, Artificial Sequence
  Synthetic 497 UL32
gucgccggauacgugggcguac

SEQ ID NO: 368 - RNA, 24 nt, Artificial Sequence
  Synthetic 481 UL29
ggcgcgugauggacauguuuaaca

SEQ ID NO: 369 - RNA, 23 nt, Artificial Sequence
  Synthetic 359 RS1
ggcccgcugcgccgcauggcggc

SEQ ID NO: 370 - RNA, 23 nt, Artificial Sequence
  Synthetic 275 RL2
ggcggugcguccgaggaagaggc

SEQ ID NO: 371 - RNA, 21 nt, Artificial Sequence
  Synthetic 360 RS1
gggcgugcugcugcuguccac

SEQ ID NO: 372 - RNA, 24 nt, Artificial Sequence
  Synthetic 443 UL22
guaaaauagcgcccgguggacgac

SEQ ID NO: 373 - RNA, 24 nt, Artificial Sequence
  Synthetic 295 RL2
gcugcggaaguccagggcgcccac

SEQ ID NO: 374 - RNA, 20 nt, Artificial Sequence
  Synthetic 276 RL2
ggugcguccgaggaagaggc

SEQ ID NO: 375 - RNA, 20 nt, Artificial Sequence
  Synthetic 607 UL6
gauaucgccgggguggccga

SEQ ID NO: 376 - RNA, 23 nt, Artificial Sequence
  Synthetic 609 UL6
gacgacgcugcuggacuuugccc

SEQ ID NO: 377 - RNA, 22 nt, Artificial Sequence
  Synthetic 484 UL29
ggaccgccugguccacguacug

SEQ ID NO: 378 - RNA, 24 nt, Artificial Sequence
  Synthetic 403 UL19
gguaggucacgacguacgagacca

SEQ ID NO: 379 - RNA, 23 nt, Artificial Sequence
  Synthetic 404 UL19
guaggucacgacguacgagacca

SEQ ID NO: 380 - RNA, 21 nt, Artificial Sequence
  Synthetic 280 RL2
gcggggccgucggugcccugg

SEQ ID NO: 381 - RNA, 22 nt, Artificial Sequence
  Synthetic 339 RS1
gccugguccagcgccacguccc

SEQ ID NO: 382 - RNA, 20 nt, Artificial Sequence
  Synthetic 602 UL6
guauaccccagcugcccgug

SEQ ID NO: 383 - RNA, 21 nt, Artificial Sequence
  Synthetic 274 RL2
gcaggaggcggcggugcgucc

SEQ ID NO: 384 - RNA, 20 nt, Artificial Sequence
  Synthetic 405 UL19
ggucacgacguacgagacca

SEQ ID NO: 385 - RNA, 21 nt, Artificial Sequence
  Synthetic 472 UL22
gcgguguaucacgcggcccgc

SEQ ID NO: 386 - RNA, 22 nt, Artificial Sequence
  Synthetic 555 UL37
gcguccgccagugcgccgugga

SEQ ID NO: 387 - RNA, 23 nt, Artificial Sequence
  Synthetic 301 RL2
gagcagggugcggcggcuccagc

SEQ ID NO: 388 - RNA, 23 nt, Artificial Sequence
  Synthetic 338 RS1
ggccugguccagcgccacguccc

SEQ ID NO: 389 - RNA, 24 nt, Artificial Sequence
  Synthetic 603 UL6
gaacgcccgggaggcggauaucgc

SEQ ID NO: 390 - RNA, 22 nt, Artificial Sequence
  Synthetic 396 UL19
gauuccggcgugcaacgccccg

SEQ ID NO: 391 - RNA, 24 nt, Artificial Sequence
  Synthetic 531 UL37
gagaagcgcggacagcggcguggc

SEQ ID NO: 392 - RNA, 21 nt, Artificial Sequence
  Synthetic 606 UL6
ggauaucgccgggguggccga

SEQ ID NO: 393 - RNA, 20 nt, Artificial Sequence
  Synthetic 495 UL29
ggcaucgcagugccacggcg

SEQ ID NO: 394 - RNA, 21 nt, Artificial Sequence
  Synthetic 468 UL22
gcgugauguccagcuccgucg

SEQ ID NO: 395 - RNA, 21 nt, Artificial Sequence
  Synthetic 277 RL2
ggaagaggcgcgggucgggcc

SEQ ID NO: 396 - RNA, 21 nt, Artificial Sequence
  Synthetic 442 UL22
gguggcuugagccagcgcguc

SEQ ID NO: 397 - RNA, 20 nt, Artificial Sequence
  Synthetic 278 RL2
ggaagaggcgcgggucgggc

SEQ ID NO: 398 - RNA, 23 nt, Artificial Sequence
  Synthetic 594 UL54
gggcccuuugacgccgagaccag

SEQ ID NO: 399 - RNA, 20 nt, Artificial Sequence
  Synthetic 279 RL2
gaagaggcgcgggucgggcc

SEQ ID NO: 400 - RNA, 24 nt, Artificial Sequence
  Synthetic 608 UL6
ggacgacgcugcuggacuuugccc

SEQ ID NO: 401 - RNA, 20 nt, Artificial Sequence
  Synthetic 492 UL29
gugucccucgcgguccaccu

SEQ ID NO: 402 - RNA, 24 nt, Artificial Sequence
  Synthetic 281 RL2
gucggugcccugggagggagacaa

SEQ ID NO: 403 - RNA, 24 nt, Artificial Sequence
  Synthetic 469 UL22
ggggcgguguaucacgcggcccgc

SEQ ID NO: 404 - RNA, 21 nt, Artificial Sequence
  Synthetic 303 RL2
gauguuccccgucuccauguc

SEQ ID NO: 405 - RNA, 24 nt, Artificial Sequence
  Synthetic 353 RS1
gcgcuaucgcgacggguacgucag

SEQ ID NO: 406 - RNA, 22 nt, Artificial Sequence
  Synthetic 432 UL19
gaugcaaacagccuguacgacg

SEQ ID NO: 407 - RNA, 21 nt, Artificial Sequence
  Synthetic 329 RS1
gagggccggucgccaggggac

SEQ ID NO: 408 - RNA, 23 nt, Artificial Sequence
  Synthetic 450 UL22
ggcggccaacccggcaagcgcgc

SEQ ID NO: 409 - RNA, 20 nt, Artificial Sequence
  Synthetic 101 UL30
guacggccccgaguucguga

SEQ ID NO: 410 - RNA, 26 nt, Artificial Sequence
  Synthetic 208 RL2
gaggccgccgaggacgucaggggggu

SEQ ID NO: 411 - RNA, 27 nt, Artificial Sequence
  Synthetic 135 UL48
gcgaggucgugaagcuggaauacgagu

SEQ ID NO: 412 - DNA, 209 bp, Artificial Sequence
  Synthetic LAT promoter
ctgcagacaggggcaccgcgcccggaaatccattaggccgcagacgaggaaaataaaattacatcacctacccacgtggtgctgtggcctgtttttgctgcgtcatctcagcctttataaaagcgggggcgcggccgtgccgatcgcgggtggtgcgaaagactttccgggcgcgtccgggtgccgcggctctccgggcccccctgcag

SEQ ID NO: 413 - DNA, 965 bp, Artificial Sequence
  Synthetic RL2 promoter
cccgggagctccgcaccaagccgctctccggagagacgatggcaggagccgcgcatatatacgcttggagccagcccgccctcacagggcgggcccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacagtctggtcgcatttgcacctcggcactcggagcgagacgcagcagccaggcagactcgggccgccccctctccgcatcaccacagaagccccgcctacgttgcgacccccagggaccctccgtccgcgaccctccagccgcatacgaccccc

SEQ ID NO: 414 - DNA, 635 bp, Artificial Sequence
  Synthetic RS1 promoter
cccgggccccgccccctgcccgttcctcgttagcatgcggaacggaagcggaaaccgccggatcgggcggtaatgagatgccatgcggggcggggcgcggacccacccgccctcgcgccccgcccatggcagatggcgcggatgggcggggccgggggttcgaccaacgggccgcggccacgggcccccggcgtgccggcgtcggggcggggtcgtgcataatggaattccgttcggggtgggcccgccgggggggcggggggccggcggcctccgctgctcctccttcccgccggcccctgggactatatgagcccgaggacgccccgatcgtccacacggagcgcggctgccgacacggatccacgacccgacgcgggaccgccagagacagaccgtcagacgctcgccgcgccgggacgccgatacgcggacgaagcgcgggagggggatcggccgtccctgtcctttttcccacccaagcatcgaccggtccgcgctagttccgcgtcgacggcgggggtcgtcggggtccgtgggtctcgccccctccccccatcgagagtccgtaggtgacctaccgtgctacgtccgccgtcgcagccgtatccccggaggatcgccccgcatcggcg

SEQ ID NO: 415 - DNA, 837 bp, Artificial Sequence
  Synthetic UL23 promoter
ggatccaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctcaatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaataccgagcgaccctgcagcgacccgcttaacagcgtcaacagcgtgccgcagatcttggtggcgtgaaactcccgcacctcttcggccagcgccttgtagaagcgcgt

SEQ ID NO: 416 - DNA, 546 bp, Artificial Sequence
  Synthetic UL27 promoter
tcaacgggcccctctttgatcactccacccacagcttcgcccagccccccaacaccgcgctgtattacagcgtcgagaacgtggggctcctgccgcacctgaaggaggagctcgcccggttcatcatgggggcggggggctcgggtgctgattgggccgtcagcgaatttcagaggttttactgttttgacggcatttccggaataacgcccactcagcgcgccgcctggcgatatattcgcgagctgattatcgccaccacactctttgcctcggtctaccggtgcggggagctcgagttgcgccgcccggactgcagccgcccgacctccgaaggtcgttaccgttacccgcccggcgtatatctcacgtacgactccgactgtccgctggtggccatcgtcgagagcgcccccgacggctgtatcggcccccggtcggtcgtggtctacgaccgagacgttttctcgatcctctactcggtcctccagcacctcgcccccaggctacctgacggggggcacgacgggcccccgtagtcccgcc

SEQ ID NO: 417 - DNA, 450 bp, Artificial Sequence
  Synthetic UL29 promoter
cgggcggcgagctgctgcgcggcgccccggccggcggcccggtttattcgcgtcggcccggccggccgggcttatggaccgccggcggccgacaggagagtgacgtagccggtgggcgtggaggggctggggcggaccggcacgcccccaggtaaagtgtacatataccaaccgcataccagacgcacccgacccggagcacctgaccgtaagcatctgtgcctctcgcagggaccccgcgttgccggccgccggggttcatcggcaccccgtggttacccggggggttgtcggtgaaggggagggattcattccccaaccccggtctccaaccctccccttgaccgtcgccgcccccccccggattttgacgctcgggagacataccttgtcgggcgtccgtcgtcgtgccgggattacctccgttcgcggaccgattgacaaaaggac

SEQ ID NO: 418 - DNA, 206 bp, Artificial Sequence
  Synthetic UL37 promoter
cccgggcctgggtccgcgaacgggatgccgggacttaagtggccgtataacaccccgcgaagacgcggggtactcgcaacgcctgcgggggtcctggagggccgcgggggatcgataattcgccgctccctacagcgcacgacagtcattcccgcccggtctcgtcgttggtctacgctgtcccccacccacgcgagccgggcgtc

SEQ ID NO: 419 - DNA, 246 bp, Artificial Sequence
  Synthetic UL38 promoter
cccgggggattgtccggatgtgcgggcagcccggacggcgtgggttgcggactttctgcggggcggcccaaatggccctttaaacgtgtgtatacggacgcgccgggccagtcggccaacacaacccaccggaggcggtagccgcgtttggctgtggggtgggtggttccgccttgcgtgagtgtcctttcgacccccccccccctccctcccccgggtcttgctaggtcgcgatctggggtcgca

SEQ ID NO: 420 - DNA, 466 bp, Artificial Sequence
  Synthetic UL39 promoter
ccgctgtcactcgttgttcgttgacccgggcgtccgccaaataaagccactgaaacccgaaacgcgagtgttgtaacgtcctttgggcgggaggaagccacaaaatgcaaatgggatacatggaaggaacacacccccgtgactcaggacatcggtgtgtccttttgggtttcactgaaactggcccgcgccccacccctgcgcgatgtggataaaaagccagcgcgggtggtttagggtaccacaggtgggtgctttggaaacttgccggtcgccgtgctcctgtgagcttgcgtccctccccggtttcctttgcgctcccgccttccggacctgctctcgcctatcttctttggctctcggtgcgattcgtcaggcagcggccttgtcgaatctcgaccccaccactcgccggacccgccgacgtcccctctcgagcccgccgaaacccgccgcgtctgttgaa

SEQ ID NO: 421 - DNA, 326 bp, Artificial Sequence
  Synthetic UL42 promoter
atttcgatggcccaactccacgcggattggtgcagcaccctgcatgcgccggtgcgggccaaccttccccccgctcattgcctcttccaaaagggtgtggcctaacgagctgggggcgtatttaatcaggctagcgcggcgggcctgccgtagtttctggctcggtgagcgacggtccggttgcttgggtcccctggctgccatcaaaaccccaccctcgcagcggcatacgccccctccgcgtcccgcacccgagaccccggcccggctgccctcaccaccgaagcccacctcgtcactgtggggtgttcccagcccgcgttggg

SEQ ID NO: 422 - DNA, 512 bp, Artificial Sequence
  Synthetic UL44 promoter
gcaggtcatcaacctcgggttggtgtttcggttttccgaggttgtcgtgtatgcggcgctagggggtgccgtgtggatttcgttggcgcaggtgctggggctccggcgtcgcctgcacaggaaggaccccggggacggggcccggttggcggcgacgcttcggggcctcttcttctccgtgtacgcgctggggtttggggtgggggtgctgctgtgccctccggggtcaacgggcgggcggtcgggcgattgatatatttttcaataaaaggcattagtcccgaagaccgccggtgtgtgatgatttcgccataacacccaaaccccggatggggcccgggtataaattccggaaggggacacgggctaccctcactatcgagggcgcttggtcgggaggccgcatcgaacgcacacccccatccggtggtccgtgtggaggtcgttttcagtgcccggtctcgctttgccgggaacgctagccgatccctcgcgagggggaggcgtcgggc

SEQ ID NO: 423 - DNA, 373 bp, Artificial Sequence
  Synthetic UL48 promoter
ggggttcattcggtgttggcgttgcgtgcctttgtttcccaatccgacggggaccgggactgggtggcggggggtgggttggacagccgccctcggttcgccttcacgtgacaggagccaatgtggggggaagtcacgaggtacggggcggcccgtgcgggttgcttaaatgcgtggtggcgaccacgggctgtcattcctcgggaacggacggggttcccgctgcccacttccccccataaggtccgtccggtcctctaacgcgtttgggggttttctcttcccgcgccgtcgggcgtcccacactctctgggcgggcggggacgatcgcatcaaaagcccgatatcgtctttcccgtatcaaccccaccca

SEQ ID NO: 424 - DNA, 401 bp, Artificial Sequence
  Synthetic UL54 promoter
accccgcccatgggtcccaattggccgtcccgttaccaagaccaacccagccagcgtatccacccccgcccgggtccccgcggaagcggaacggggtatgtgatatgctaattaaatacatgccacgtacttatggtgtctgattggtccttgtctgtgccggaggtggggcgggggccccgcccggggggcggaacgaggaggggtttgggagagccggccccggcaccacgggtataaggacatccaccacccggccggtggtggtgtgcagccgtgttccaaccacggtcacgcttcggtgcctctccccgattcgggcccggtcgctcgctaccggtgcgccaccaccagaggccatatccgacaccccagccccgacggcagccgacagcccggtc

SEQ ID NO: 425 - DNA, 485 bp, Artificial Sequence
  Synthetic US6 promoter
ctgcttgagctcctgcgtcgtacgtgccgcgggtgggggcgttaccatccctacatggacccagttgtcgtataatttcccccccccccccccttctccgcgtgggtgatgtcgggtccaaactcccgacaccaccagctggcatggtataaatcaccggtgcgccccccaaaccatgtccggcagggggatgggggggcgaatgcggagggcacccaacaacaccgggctaaccaggaaatccgtggccccggcccccaataaagatcgcggtagcccggccgtgtgacactatcgtccataccgaccacaccgacgaatcccccaagggggaggggccattttacgaggaggaggggtataacaaagtctgtctttaaaaagcaggggttagggagttgttcggtcataagcttcagcgcgaacgaccaactaccccgatcatcagttatccttaaggtctcttttgtgtggtgcgttccggt

(19) United States
(12) Patent Application Publication
(10) Pub. No.: US 2020/0392208 A1
(43) Pub. Date: Dec. 17, 2020

(54) CRISPR/CAS-RELATED METHODS AND COMPOSITIONS FOR TREATING HERPES SIMPLEX VIRUS (HSV) RELATED KERATITIS

(71) Applicant: EDITAS MEDICINE, INC., Cambridge, MA (US)

(72) Inventors: Benjamin Aryeh Diner, Somerville, MA (US); Christopher M. Owens, Belmont, MA (US); Ari Friedland, Cambridge, MA (US)

(73) Assignee: EDITAS MEDICINE, INC., Cambridge, MA (US)

(21) Appl. No.: 16/944,584

(22) Filed: Jul. 31, 2020

Related U.S. Application Data
(63) Continuation of application No. PCT/US19/16391, filed on Feb. 1, 2019.
(60) Provisional application No. 62/663,720, filed on Apr. 27, 2018.
(60) Provisional application No. 62/625,114, filed on Feb. 1, 2018.

(51) Int. Cl. C07K 16/08; A61P 17/00; A61K 39/245; A61P 31/22; A61K 39/085; A61P 29/00; A61P 31/14

(52) U.S. Cl. CPC C07K 16/087; A61P 17/00; A61K 39/245; A61P 31/14; A61K 39/085; A61P 29/00; A61P 31/22

(57) ABSTRACT

CRISPR/CAS-related systems, compositions and methods for editing HSV-1 genes in human cells are described, as are cells and compositions including cells edited according to the same. Methods for treating HSV-related keratitis using the said CRISPR/CAS-related systems, compositions and methods are also described.

### CROSS-REFERENCE TO RELATED APPLICATIONS

This application is a Continuation of International Patent Application No. PCT/US19/016391 filed on Feb. 1, 2019, which claims priority to U.S. Provisional Application No. 62/625,114 filed on Feb. 1, 2018, the content of each of which is incorporated by reference in its entirety and to each of which priority claims.

### SEQUENCE LISTING

The specification further incorporates by reference the Sequence Listing submitted herewith via EFS on Jul. 31, 2020. Pursuant to 37 C.F.R. § 1.52(e)(5), the Sequence Listing text file, identified as 084177_0240_SL.txt, is 90,699 bytes and was created on Jul. 31, 2020. The Sequence Listing electronically filed herewith, does not extend beyond the scope of the specification and thus does not contain new matter.

### FIELD

The present disclosure relates to CRISPR/Cas-related methods and components for editing a target nucleic acid sequence, or modulating expression of a target nucleic acid sequence, and applications thereof in connection with herpes simplex virus type 1 (HSV-1). The present disclosure also relates to methods for treating HSV-related keratitis using CRISPR/Cas-related components.

### BACKGROUND

CRISPRs (Clustered Regularly Interspaced Short Palindromic Repeats) evolved in bacteria and archaea as an adaptive immune system to defend against viral attack. Upon exposure to a virus, short segments of viral DNA are integrated into the CRISPR locus. RNA is transcribed from a portion of the CRISPR locus that includes the viral sequence. That RNA, which contains sequence complementary to the viral genome, mediates targeting of a Cas9 protein to a target sequence in the viral genome. The Cas9 protein, in turn, cleaves and thereby silences the viral target.

Recently, the CRISPR/Cas system has been adapted for genome editing in eukaryotic cells. The introduction of site-specific double strand breaks (DSBs) allows for target sequence alteration through endogenous DNA repair mechanisms, for example non-homologous end-joining (NHEJ) or homology-directed repair (HDR).

Herpes simplex virus-related keratitis annually impacts over 58,000 individuals in the US (Farooq et al., 2012; Survey of Ophthalmology 57: 448-62). Three major classes of keratitis have been recognized: epithelial, stromal, and endothelial keratitis. Each debilitating episode of keratitis lasts 17 to 28 days, with longer duration associated with individuals with recurrent episodes. Recurrent stromal keratitis is associated with a high risk of blindness. About 1.5% of HSV keratitis patients experience devastating vision loss, BCVA >20/200, each year. By adulthood, up to 80% of the population in the United States is infected with HSV-1 (Liesegang, 2001; Conea 20(1):1-13). Incidence of HSV keratitis in adults may increase due to increasing numbers of baby boomers reaching age >60 and the severity of ocular manifestations is also known to increase with age. The annual incidence of new cases of HSV keratitis in the US is projected to be >33,000 individuals, with ˜2000 having stromal keratitis.

Recurrent herpes simplex virus (HSV) keratitis, a result of latent virus reactivation within the trigeminal ganglia (TG), is considered the leading cause of infectious corneal blindness worldwide. Current standard of care, such as antivirals, fails to block latent virus reactivation or immune responses to viral proteins associated with devastating vision loss, and for many patients, is contraindicated. Therefore, there remains a need for treatment of HSV-related keratitis.

### SUMMARY

The presently disclosed subject matter relates to RNA-guided nuclease-related, e.g., CRISPR/Cas-related, methods, genome editing systems, and compositions for editing a target nucleic acid sequence, or modulating expression of a target nucleic acid sequence, and applications thereof in connection with herpes simplex virus type 1 (HSV-1). The presently disclosed subject matter also provides genome editing systems, compositions, vectors, and methods of treating HSV-related keratitis using CRISPR/Cas-related components to edit a target HSV-1 gene.

In one aspect, the presently disclosed subject matter relates to a genome editing system including a first gRNA molecule which includes a first targeting domain that is complementary with a first target sequence of a first HSV-1 gene, a second gRNA molecule which includes a second targeting domain that is complementary with a second target sequence of a second HSV-1 gene, and an RNA-guided nuclease.

In another aspect, the presently disclosed subject matter relates to a composition including a first gRNA molecule which includes a first targeting domain that is complementary with a first target sequence of a first HSV-1 gene, a second gRNA molecule which includes a second targeting domain that is complementary with a second target sequence of a second HSV-1 gene, and an RNA-guided nuclease. In another aspect, the presently disclosed subject matter relates to a vector including a polynucleotide encoding (a) a first gRNA molecule which includes a first targeting domain that is complementary with a first target sequence of a first HSV-1 gene, (b) a second gRNA molecule which includes a second targeting domain that is complementary with a second target sequence of a second HSV-1 gene, and (c) an RNA-guided nuclease.

In various non-limiting embodiments, the first HSV-1 gene is different from the second HSV-1 gene.

In various non-limiting embodiments, the first HSV-1 gene is the same as the second HSV-1 gene.

In various non-limiting embodiments, each of the first and second HSV-1 genes is selected from the group consisting of immediate early HSV-1 genes, early HSV-1 genes, and late HSV-1 genes.

In various non-limiting embodiments, each of the first and second HSV-1 genes are selected from the group consisting of immediate early HSV-1 genes, early HSV-1 genes, and late HSV-1 genes.

In various non-limiting embodiments, the immediate-early HSV-1 genes are selected from the group consisting of a RL2 gene, a RS1 gene, a UL54 gene, a US1 gene, a US1.5 gene, and a US12 gene. In certain embodiments, the immediate-early HSV-1 genes are selected from the group consisting of a RL2 gene, a RS1 gene, and a UL54 gene.

In various non-limiting embodiments, the early HSV-1 genes are selected from the group consisting of a UL5 gene, a UL8 gene, a UL9 gene, a UL23 gene, a UL29 gene, a UL30 gene, a UL42 gene, and a UL52 gene. In certain embodiments, the early HSV-1 gene is a UL29 gene.

In various non-limiting embodiments, the late HSV-1 genes are selected from the group consisting of a UL1 gene, a UL6 gene, a UL15 gene, a UL16 gene, a UL18 gene, a UL19 gene, a UL22 gene, a UL26 gene, a UL26.5 gene, a UL27 gene, a UL28 gene, a UL31 gene, a UL32 gene, a UL33 gene, a UL34 gene, a UL35 gene, a UL36 gene, a UL37 gene, a UL38 gene, a UL48 gene, a UL49.5 gene, and a US6 gene. In certain embodiments, the late HSV-1 genes are selected from the group consisting of a UL6 gene, a UL15 gene, a UL19 gene, a UL22 gene, a UL32 gene, a UL33 gene, a UL37 gene, and a UL48 gene.

In various non-limiting embodiments, the first HSV-1 gene is a late HSV-1 gene, and the second HSV-1 gene is an immediate early HSV-1 gene, an early HSV-1 gene, or a late HSV-1 gene. In certain embodiments, the first HSV-1 gene is a late HSV-1 gene, and the second HSV-1 gene is an immediate early HSV-1 gene. In certain embodiments, the first HSV-1 gene is a UL48 gene, and the second HSV-1 gene is a RL2 gene.

In various non-limiting embodiments, the first HSV-1 gene is a late HSV-1 gene, and the second HSV-1 gene is a late HSV-1 gene. In certain embodiments, the first HSV-1 gene is a late HSV-1 gene, and the second HSV-1 gene is an early HSV-1 gene. In certain embodiments, the first HSV-1 gene is an early HSV-1 gene, and the second HSV-1 gene is an early HSV-1 gene. In certain embodiments, the first HSV-1 gene is an early HSV-1 gene, and the second HSV-1 gene is an immediate early HSV-1 gene. In certain embodiments, the first HSV-1 gene is an immediate early HSV-1 gene, and the second HSV-1 gene is an immediate early HSV-1 gene.

In various non-limiting embodiments, the first targeting domain includes a nucleotide sequence selected from SEQ ID NOs: 1-411, and the second targeting domain includes a nucleotide sequence selected from SEQ ID NOs: 1-411. In various non-limiting embodiments, the first targeting domain includes a nucleotide sequence selected from SEQ ID NOs: 1-54, 410, and 411, and the second targeting domain includes a nucleotide sequence selected from SEQ ID NOs: 1-54, 410, and 411. In various non-limiting embodiments, the first targeting domain includes a nucleotide sequence selected from SEQ ID NOs: 1-25, 410, and 411, and the second targeting domain includes a nucleotide sequence selected from SEQ ID NOs: 1-25, 410, and 411. In various non-limiting embodiments, the first targeting domain includes a nucleotide sequence selected from SEQ ID NOs: 1-14, 410, and 411, and the second targeting domain includes a nucleotide sequence selected from SEQ ID NOs: 1-14, 410, and 411. In various non-limiting embodiments, the first targeting domain includes the nucleotide sequence set forth in SEQ ID NO: 410, and second targeting domain includes the nucleotide sequence set forth in SEQ ID NO: 411.

In various non-limiting embodiments, the system, composition or vector further includes a Cas9 molecule and a second Cas9 molecule that are configured to form complexes with the first and second gRNAs. In certain embodiments, at least one of the first and second Cas9 molecules includes an S. pyogenes Cas9 molecule or an S. aureus Cas9 molecule. In certain embodiments, at least one of the first and second Cas9 molecules includes a wild-type Cas9 molecule, a mutant Cas9 molecule, or a combination thereof. In certain embodiments, the mutant Cas9 molecule includes a D10A mutation.

In certain embodiments, the vector is a viral vector. In certain embodiments, the vector is an Adeno-associated virus (AAV) vector. In certain embodiments, the said AAV vector is a serotype 1, 2, 3, 4, 5, 6, 7, 8 or 9 vector.

In certain embodiments, the system, composition or vector further includes a third gRNA molecule comprising a third targeting domain that is complementary with a third target sequence of a third HSV-1 gene. In certain embodiments, the system, composition or vector further includes a fourth gRNA molecule comprising a fourth targeting domain that is complementary with a fourth target sequence of a fourth HSV-1 gene. In certain embodiments, the system, composition or vector further includes a fifth gRNA molecule comprising a fifth targeting domain that is complementary with a fifth target sequence of a fifth HSV-1 gene. One or two or all of the third, fourth and fifth HSV-1 genes can be the same as or different from one or both of the first and second HSV-1 genes.

In another aspect, the presently disclosed subject matter relates to a method of altering a first HSV-1 gene and a second HSV-1 gene in a cell, including administrating to the cell one of: (i) a genome editing system including a first gRNA molecule that includes a first targeting domain that is complementary with a first target sequence of the first HSV-1 gene, a second gRNA molecule that includes a second targeting domain that is complementary with a second target sequence of the second HSV-1 gene, and at least an RNA-guided nuclease; (ii) a genome editing system including a first polynucleotide encoding a first gRNA molecule that includes a first targeting domain that is complementary with a first target sequence of the first HSV-1 gene, a second polynucleotide encoding a second gRNA molecule that includes a second targeting domain that is complementary with a second target sequence of the second HSV-1 gene, and a third polynucleotide encoding an RNA-guided nuclease; (iii) a composition including a first gRNA molecule that includes a first targeting domain that is complementary with a first target sequence of the first HSV-1 gene, a second gRNA molecule that includes a second targeting domain that is complementary with a target sequence of second HSV-1 gene, and at least an RNA-guided nuclease; and (iv) a vector including a polynucleotide encoding (a) a first gRNA molecule which includes a first targeting domain that is complementary with a first target sequence of a first HSV-1 gene, (b) a second gRNA molecule which includes a second targeting domain that is complementary with a second target sequence of a second HSV-1 gene, and (c) an RNA-guided nuclease.

In another aspect, the presently disclosed subject matter relates to a method for treating or preventing a HSV-related disease in a subject, including administrating to the subject one of: (i) a genome editing system including a first gRNA molecule that includes a first targeting domain that is complementary with a first target sequence of the first HSV-1 gene, a second gRNA molecule that includes a second targeting domain that is complementary with a second target sequence of the second HSV-1 gene, and at least an RNA-guided nuclease; (ii) a genome editing system including a first polynucleotide encoding a first gRNA molecule that includes a first targeting domain that is complementary with a first target sequence of the first HSV-1 gene, a second polynucleotide encoding a second gRNA molecule that includes a second targeting domain that is complementary with a second target sequence of the second HSV-1 gene, and a third polynucleotide encoding an RNA-guided nuclease; (iii) a composition including a first gRNA molecule that includes a first targeting domain that is complementary with a first target sequence of the first HSV-1 gene, a second gRNA molecule that includes a second targeting domain that is complementary with a target sequence of second HSV-1 gene, and at least an-RNA guided nuclease; and (iv) a vector including a polynucleotide encoding (a) a first gRNA molecule which includes a first targeting domain that is complementary with a first target sequence of a first HSV-1 gene, (b) a second gRNA molecule which includes a second targeting domain that is complementary with a second target sequence of a second HSV-1 gene, and (c) an RNA-guided nuclease. In certain embodiments, the HSV-related disease is a recurrent HSV-1 ocular keratitis. In certain embodiments, the HSV-related disease is a recurrent HSV-2 ocular keratitis.

In certain embodiments, the subject is a human subject. In certain embodiments, the administration is initiated prior to the subject is exposed to a virus. In certain embodiments, the administration is initiated prior to the HSV-related disease onset. In certain embodiments, the administration is initiated in an advanced stage of the HSV-related disease. In certain embodiments, the administration is initiated in an early stage of the HSV-related disease.

### BRIEF DESCRIPTION OF THE DRAWINGS

The accompanying drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides can be depicted as linear sequences, or as schematic two- or three-dimensional structures; these depictions are intended to be illustrative rather than limiting or binding to any particular model or theory regarding their structure.

FIG. 1 depicts gRNA development scheme.

FIG. 2 depicts the scheme of rabbit HSV-1 ocular keratitis reactivation study.

FIG. 3 shows tear swab plaque score from the rabbit HSV-1 ocular keratitis reactivation study.

FIG. 4 shows Slit Lamp Examination (SLE) corneal lesion scores from the rabbit HSV-1 ocular keratitis reactivation study.

FIG. 5 shows HSV-1 copy number assessment per rabbit trigeminal ganglion (TG) from the rabbit HSV-1 ocular keratitis reactivation study.

FIG. 6 depicts the scheme of primary in vitro cell-based gRNA screen.

FIG. 7 shows gRNA hits selected from gRNA screen.

FIG. 8 shows EC₅₀ of the selected RNP.

FIG. 9 depicts the differences between gene editing and standard of care in treating HSV-related keratitis.

FIG. 10 shows the results of virus inhibition assay for 55 candidate gRNAs (SEQ ID NOs.: 1-54) targeting 12 essential HSV genes.

FIG. 11 shows the results of virus inhibition assay. This assay was performed to test the ability of 55 candidate gRNAs, after complexed to form RNPs, in inhibition of viral replication.

FIG. 12 shows the results of virus inhibition assay for 24 gRNAs (SEQ ID NOs: 1-12, and 14-25) for each of the 12 essential HSV-1 genes, after complexed to form RNPs.

FIG. 13 shows the results of the intracellular BAC cutting assay for screening the 24 gRNAs. HSV-1 production from the BAC was measured by ddPCR.

FIG. 14 shows the 12 gRNAs (SEQ ID NOs.: 1-12) and their ability in reducing viral replication as compared to a scrambled gRNA, measured by an intracellular BAC cutting assay.

FIG. 15 shows the effects of RNP transfection on BAC copy numbers per U502 genome. Optimal plasmid dose for pairwise combination screen was determined based on this result. For each plasmid doses on the bar figure (left panel), the left bar represents results of gRNA 480, middle bar represents results of gRNA 570, right bar represents results of gRNA 417.

FIG. 16 shows the exemplary gRNA combinations tested.

FIG. 17 shows the results for assessing multiplexing of gRNAs. Vero cells were nucleofected with plasmids expressing SaCas9 and selected gRNAs. Cells were then challenged with WT HSV-1. HSV copies were measured in the supernatant collected from the culture. C2: 2 gRNAs combination; C3: 3 gRNAs combination; C4: 4 gRNAs combination; C5: 5 gRNAs combination.

FIG. 18 is a representative image showing the in-situ hybridization data for mCherry transgene transcripts at TG in rabbits received AAV through corneal intrastromal injection. Arrows indicate the positive stain for mCherry transgene transcripts.

FIG. 19 is a representative image showing the in-situ hybridization data for mCherry transgene transcripts at TG in rabbits received AAV through direct TG injection. Arrows indicate the positive stain for mCherry transgene transcripts.

FIG. 20 shows the results of tear swab plaque assay.

FIG. 21 shows the representative images of plaque assay.

FIGS. 22A-22B shows (A) HSV genome measured in tear swaps by qPCR and (B) HSV infectivity measured in tear swaps by plaque assay. Each data point represents % swabs positive per treatment group eye.

FIG. 23 shows the area under the curve (AUC) analysis for measuring HSV genome (top panels), and HSV plaques (bottom panels).

FIG. 24 shows the measured AUC for HSV genome and HSV infectivity in each group.

FIG. 25 shows the levels of viral genomes measured in rabbit corneas and TGs.

FIG. 26 shows the levels of SaCas9 expression in rabbit corneas and TGs.

FIG. 27 shows the correlation between AAV genomes and Cas9 expression in rabbit corneas.

FIG. 28 shows the results of in situ hybridization (ISH)-based detection of AAV and productive or latent HSV in corneas and TGs

FIG. 29 shows the results of the slit lamp examination (SLE), which allows the observation and scoring of HSV-induced pathology.

FIG. 30 shows the presently disclosed exemplary 2-gRNA combinations. gRNAs are identified by their ID numbers disclosed in Table 2. “Cnb” column shows combination numbers. “gRNA” columns show gRNA ID numbers.

FIG. 31 shows the presently disclosed exemplary 3-gRNA combinations. gRNAs are identified by their ID numbers disclosed in Table 2. “Cnb” column shows combination numbers. “gRNA” columns show gRNA ID numbers.

FIG. 32 shows the presently disclosed exemplary 4-gRNA combinations. gRNAs are identified by their ID numbers disclosed in Table 2. “Cnb” column shows combination numbers. “gRNA” columns show gRNA ID numbers.

FIG. 33 shows the presently disclosed exemplary 5-gRNA combinations. gRNAs are identified by their ID numbers disclosed in Table 2. “Cnb” column shows combination numbers. “gRNA” columns show gRNA ID numbers.

### DETAILED DESCRIPTION

### Definitions and Abbreviations

Unless otherwise specified, each of the following terms has the meaning associated with it in this section.

The indefinite articles “a” and “an” refer to at least one of the associated noun, and are used interchangeably with the terms “at least one” and “one or more.” For example, “a module” means at least one module, or one or more modules.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The conjunctions “or” and “and/or” are used interchangeably as non-exclusive disjunctions.

The phrase “consisting essentially of” means that the species recited are the predominant species, but that other species can be present in trace amounts or amounts that do not affect structure, function or behavior of the subject composition. For instance, a composition that consists essentially of a particular species will generally comprise 90%, 95%, 96%, or more of that species.

“Domain” is used to describe a segment of a protein or nucleic acid. Unless otherwise indicated, a domain is not required to have any specific functional property.

An “indel” is an insertion and/or deletion in a nucleic acid sequence. An indel can be the product of the repair of a DNA double strand break, such as a double strand break formed by a genome editing system of the present disclosure. An indel is most commonly formed when a break is repaired by an “error prone” repair pathway such as the NHEJ pathway described below.

“Gene conversion” refers to the alteration of a DNA sequence by incorporation of an endogenous homologous sequence (e.g. a homologous sequence within a gene array). “Gene correction” refers to the alteration of a DNA sequence by incorporation of an exogenous homologous sequence, such as an exogenous single- or double stranded donor template DNA. Gene conversion and gene correction are products of the repair of DNA double-strand breaks by HDR pathways such as those described below.

Indels, gene conversion, gene correction, and other genome editing outcomes are typically assessed by sequencing (most commonly by “next-gen” or “sequencing-by-synthesis” methods, though Sanger sequencing can still be used) and are quantified by the relative frequency of numerical changes (e.g., ±1, ±2 or more bases) at a site of interest among all sequencing reads. DNA samples for sequencing can be prepared by a variety of methods known in the art, and can involve the amplification of sites of interest by polymerase chain reaction (PCR), the capture of DNA ends generated by double strand breaks, as in the GUIDEseq process described in Tsai et al. (Nat. Biotechnol. 34(5): 483 (2016), incorporated by reference herein) or by other means well known in the art. Genome editing outcomes can also be assessed by in situ hybridization methods such as the FiberComb™ system commercialized by Genomic Vision (Bagneux, France), and by any other suitable methods known in the art.

“Alt-HDR,” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA. In a normal cell, cHDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded.

Unless indicated otherwise, the term “HDR” as used herein encompasses both canonical HDR and alt-HDR.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

“Replacement” or “replaced,” when used with reference to a modification of a molecule (e.g. a nucleic acid or protein), does not require a process limitation but merely indicates that the replacement entity is present.

“Subject” means a human or non-human animal. A human subject can be any age (e.g., an infant, child, young adult, or adult), and can suffer from a disease, or can be in need of alteration of a gene. Alternatively, the subject can be an animal, which term includes, but is not limited to, mammals, birds, fish, reptiles, amphibians, and more particularly non-human primates, rodents (such as mice, rats, hamsters, etc.), rabbits, guinea pigs, dogs, cats, and so on. In certain embodiments of this disclosure, the subject is livestock, e.g., a cow, a horse, a sheep, or a goat. In certain embodiments, the subject is poultry.

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” refer to the prevention of a disease in a mammal, e.g., in a human, including (a) avoiding or precluding the disease; (b) affecting the predisposition toward the disease; or (c) preventing or delaying the onset of at least one symptom of the disease.

The term “keratitis” or “ocular keratitis” refers to a condition in which the eye's cornea, the clear dome on the front surface of the eye, becomes inflamed. In certain embodiments, the ocular keratitis is HSV-1 ocular keratitis. In certain embodiments, the ocular keratitis is HSV-2 ocular keratitis.

A “Kit” refers to any collection of two or more components that together constitute a functional unit that can be employed for a specific purpose. By way of illustration (and not limitation), one kit according to this disclosure can include a guide RNA complexed or able to complex with an RNA-guided nuclease, and accompanied by (e.g. suspended in, or suspendable in) a pharmaceutically acceptable carrier. The kit can be used to introduce the complex into, for example, a cell or a subject, for the purpose of causing a desired genomic alteration in such cell or subject.

The components of a kit can be packaged together, or they can be separately packaged. Kits according to this disclosure also optionally include directions for use (DFU) that describe the use of the kit e.g., according to a method of this disclosure. The DFU can be physically packaged with the kit, or it can be made available to a user of the kit, for instance by electronic means.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 1, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence can be encoded by either DNA or RNA, for example in gRNA targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The term “variant” refers to an entity such as a polypeptide, polynucleotide or small molecule that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity.

As used herein, the term “promoter” refers to a region (i.e., a DNA sequence) of a genome that initiates the transcription of a gene.

### Overview

Provided herein are compositions, systems, and methods for the treatment and prevention of HSV-related ocular infections, including but not limited to HSV-related keratitis, HSV-related retinitis, HSV-related encephalitis and HSV-related meningitis. In certain embodiments, the methods disclosed herein involve gene editing approaches using an RNA-guided nuclease to target HSV-1 genomes. In certain embodiments, the HSV-1 genome is a latent HSV-1 genome. In certain embodiments, the HSV-1 genome is a reactive HSV-1 genome. In certain embodiments, the HSV-1 genome is a shedding HSV-1 genome. In certain embodiments, the HSV-1 genome is a replicative HSV-1 genome. In certain embodiments, the HSV-1 genome is an active HSV-1 genome. In certain embodiments, the gene editing approach comprises delivering at least two gRNAs targeting at least one HSV-1 gene, including, but not limited to, immediate-early, early, and/or late HSV-1 genes.

### HSV-Related Ocular Disease

HSV infections, e.g., HSV-1 and/or HSV-2 infections of the eye, either primary or reactivation infections, are called HSV-related ocular disease. HSV-related ocular disease most commonly causes infection of the anterior chamber of the eye, known as keratitis, stromal keratitis and/or disciform keratitis. HSV-related ocular disease may, more rarely, cause infection of the posterior chamber of the eye, known as retinitis. HSV-1 keratitis is acutely painful and unpleasant. It may, in rare instances, cause scarring, secondary infection with bacterial pathogens and rarely, blindness. HSV-related retinitis is a rare manifestation of HSV-related ocular disease but carries a much higher risk of permanent visual damage.

Reactivation infections occur in the eye via anterograde transport of the virus into the eye from the trigeminal ganglion, along the ophthalmic branch of the trigeminal nerve (the fifth cranial nerve) and into the eye. Re-activation of the virus may also occur from within the cornea. Latency within the trigeminal ganglion is established via one of two mechanisms. First, HSV-1 or HSV-2 can travel via retrograde transport along the trigeminal nerve from the eye (after an eye infection) into the trigeminal ganglion. Alternatively, it can spread to the trigeminal ganglion via hematogenous spread following infection of the oral mucosa, genital region, or other extraocular site. After establishing latent infection of the trigeminal ganglion, at any time, particularly in the event of an immunocompromised host, the virus can re-establish infection by traveling anterograde along the trigeminal nerve and into the eye.

When ocular herpes affects the posterior chamber of the eye, it causes retinitis. In adults, HSV-1 is responsible for the majority of cases of HSV-retinitis (Pepose et al., Ocular Infection and Immunity 1996; Mosby 1155-1168). In neonates and children, HSV-2 is responsible for the majority of cases of HSV-retinitis (Pepose et al., Ocular Infection and Immunity 1996; Mosby 1155-1168). HSV-related retinitis can lead to acute retinal necrosis (ARN), which will destroy the retina within 2 weeks without treatment (Banerjee and Rouse, Human Herpesviruses 2007; Cambridge University Press, Chapter 35). Even with treatment, the risk of permanent visual damage following ARN is higher than 50% (Roy et al., Ocular Immunology and Inflammation 2014; 22(3):170-174).

Keratitis is the most common form of ocular herpes. HSV keratitis can manifest as dendritic keratitis, stromal keratitis, blepharitis and conjunctivitis. HSV-1 is responsible for the majority of HSV-associated keratitis, accounting for 58% of cases (Dawson et. al., Survey of Ophthalmology 1976; 21(2): 121-135). HSV-2 accounts for the remainder of HSV-associated keratitis cases, or approximately 42% of cases. In the U.S., there are approximately 48,000 cases of recurrent or primary HSV-related keratitis infections annually (Liesegang et. al., 1989; 107(8): 1155-1159). Of all cases of HSV-related keratitis, approximately 1.5-3% of subjects experience severe, permanent visual impairment (Wilhelmus et. al., Archives of Ophthalmology 1981; 99(9): 1578-82). The risk to a subject of permanent visual damage due to HSV-related ocular disease increases with increasing numbers of ocular related HSV-reactivations.

Overall, stromal keratitis represents approximately 15% of keratitis cases and is associated with the highest risk of permanent visual damage from keratitis. Stromal keratitis results in scarring and irregular astigmatism. Previous ocular HSV infection increases the risk for developing stromal infection, which means that subjects who have had a prior ocular HSV infection have an increased risk for permanent visual damage on reactivation. In children, stromal keratitis represents up to 60% of all keratitis cases. Therefore, children are particularly at risk for permanent visual damage from HSV-associated keratitis. A retrospective study in the U.S. from 1950-1982 found that there are approximately 2.6 new or recurrent stromal keratitis cases per 100,000 person years, or approximately 8,000 cases of stromal keratitis annually (Liesegang et. al., 1989; 107(8): 1155-1159). A more recent study in France in 2002 estimated the incidence of new or recurrent stromal keratitis cases to be 9.6 per 100,000 (Labetoulle et al., Ophthalmology 2005; 112(5):888-895). The incidence of HSV-associated keratitis may be increasing in the developed world (Farooq and Shukla 2012; Survey of Ophthalmology 57(5): 448-462).

The genome editing systems, compositions and methods described herein can be used for the treatment, prevention and/or reduction of HSV-1 and/or HSV-2 ocular infections, including but not limited to HSV-1 stromal keratitis, HSV-1 dendritic keratitis, HSV-1 blepharitis, HSV-1 conjunctivitis, HSV-1 retinitis, HSV-2 stromal keratitis, HSV-2 dendritic keratitis, HSV-2 blepharitis, HSV-2 conjunctivitis, and HSV-2 retinitis.

### Herpes Simplex Virus Type 1

Herpes simplex virus type 1 (HSV-1) is a ubiquitous and highly contagious pathogen. HSV-1 is contained within an icosahedral particle, and enters the host via infection of epithelial cells within the skin and mucous membranes. HSV-1 produces immediate early genes within the epithelial cells, which encode enzymes and binding proteins necessary for viral synthesis. After primary infection, the virus travels up sensory nerve axons via retrograde transport to the sensory dorsal root ganglion (DRG). Within the DRG, it establishes a latent infection. The latent infection persists for the lifetime of the host. Within the DRG cell, the virus uncoats, viral DNA is transported into the nucleus, and key viral RNAs associated with latency are transcribed (including the LAT RNAs).

Most subjects develop the HSV-1 infection during childhood. During primary infection, the virus infects cells of the oropharynx and ano-genital region, causing painful vesicles in the affected region. HSV-1 infection persists for the lifetime of the host, and can cause permanent neurologic sequelae and blindness.

Reactivation of HSV-1 infections occurs in the oropharynx and ano-genital region of eye and central nervous system, and can have severe and damaging HSV manifestation, leading to blindness and permanent neurologic disability.

Methods to Treat, Prevent and/or Reduce HSV-Related Ocular Keratitis

Disclosed herein are the approaches to treat, prevent, and/or reduce HSV-related ocular keratitis, using the systems, compositions, vectors, and methods described herein. HSV-related ocular infection may be caused by an HSV-1 and/or HSV-2 infection. The methods, systems, vectors, and compositions disclosed herein can be used to treat, prevent, and/or reduce HSV-1 infection, HSV-2 infection, or both HSV-1 and HSV-2 infections. In certain embodiments, the HSV-related ocular keratitis is recurrent ocular keratitis, including, but not limited to, HSV-1 recurrent ocular keratitis and HSV-2 recurrent ocular keratitis.

Recurrent ocular keratitis is a result of latent virus reactivation within the trigeminal ganglia (TG). HSV-1 or HSV-2 relies on essential viral genes, such as immediate-early, early and late genes, for infection, proliferation and assembly. A gene editing approach using CRISP/Cas9 to target latent HSV-1 genomes, knocking out viral genes, e.g., essential viral genes, individually or in combination can limit viral resistance and treat recurrent HSV ocular infection. As the HSV-1 or

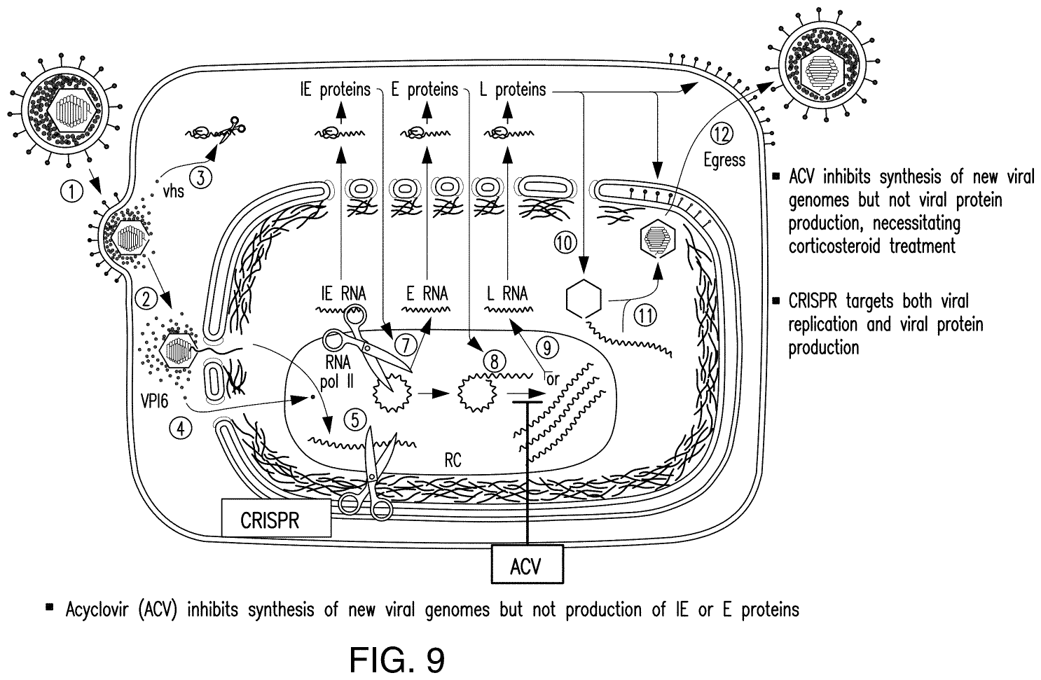

HSV-2 virus establishes latency in discrete, localized regions within the body, targeted knockout at the region of latency (e.g., the trigeminal dorsal root ganglion, e.g., the cervical dorsal root ganglia, e.g., the sacral dorsal root ganglia), can reduce or eliminate latent infection by disabling the HSV-1 and/or HSV-2 virus. Non-limiting differences between gene editing and standard of care in treating HSV-related diseases are shown in FIG. 9.

Described herein are the approaches to treat ocular keratitis by editing viral genome and knocking out one or more HSV-1 genes (e.g., essential HSV-1 viral genes). In certain embodiments, the viral genome is a latent viral genome. In certain embodiments, the viral genome is a reactive viral genome. In certain embodiments, the viral genome is a shedding viral genome. In certain embodiments, the viral genome is a replicative viral genome. In certain embodiments, the viral genome is an active viral genome. In certain embodiments, the approaches disclosed herein are for treatment of recurrent ocular keratitis by editing latent viral genome and knocking out one or more HSV-1 genes (e.g., essential HSV-1 viral genes). Methods described herein include knocking out one or more HSV-1 gene. In certain embodiments, the method comprises knocking out one HSV-1 gene, which can be an essential HSV-1 viral gene or a non-essential HSV-1 viral gene. In certain embodiments, the HSV-1 gene is an essential HSV-1 viral gene. In certain embodiments, the method comprises knocking out two or more HSV-1 genes. In certain embodiments, the method comprises knocking out two HSV-1 genes, e.g., two essential HSV-1 viral genes, two non-essential HSV-1 viral genes, or one essential HSV-1 viral gene and one non-essential HSV-1 viral gene.

“Essential viral gene” refers to a viral gene that is essential in certain but not necessarily all circumstances for the survival, replication, and/or propagation of the virus in vivo. “Essential HSV-1 gene” refers to an HSV-1 gene that is essential in certain but not all circumstances for the survival replication, and/or propagation of HSV-1 virus in vivo. Non-limiting examples of essential HSV-1 genes include RL2 gene, RS1 gene, UL54 gene, US1 gene, US1.5 gene, US12 gene, UL5 gene, UL8 gene, UL9 gene, UL23 gene, UL29 gene, UL30 gene, UL42 gene, UL52 gene, UL1 gene, UL6 gene, UL15 gene, UL16 gene, UL18 gene, UL19 gene, UL22 gene, UL26 gene, UL26.5 gene, UL27 gene, UL28 gene, UL31 gene, UL32 gene, UL33 gene, UL34 gene, UL35 gene, UL36 gene, UL37 gene, UL38 gene, UL48 gene, UL49.5 gene, and US6 gene.

Non-limiting examples of HSV-1 genes include immediate-early HSV-1 genes (or “IE gene”), early HSV-1 genes (or “E gene”), and late HSV-1 genes (or “L gene”).

“Immediate-early gene” or “IE gene” or “a gene” refers to genes that are activated and transcribed immediately after viral infection, in the absence of de novo protein synthesis. The IE proteins encoded by the corresponding IE genes are responsible for regulating viral gene expression during subsequent phases of the replication cycle (Sanfilippo et al., Journal of Virology (2018); 92(2)224-39). The IE genes act in part to up-regulate the expression of the early genes. Non-limiting examples of immediate-early genes of HSV-1 include RL2 gene, RS1 gene, UL54 gene, US1 gene, US1.5 gene, and US12 gene. In certain embodiments, the immediate-early genes are selected from the group consisting of a RL2 gene, a RS1 gene, and a UL54 gene.

“Early gene” or “E gene” or “β gene” refers to genes that encode proteins required for viral DNA synthesis. The expression of early genes is regulated by the IE proteins (Pesola et al., Journal of Virology (2005); 79(23):14516-25). In HSV-1, the function of several early genes is to turn off the expression of the immediate-early gene and to induce the expression of the late genes. Non-limiting examples of early genes of HSV-1 include, but not limited to UL5 gene, UL8 gene, UL9 gene, UL23 gene, UL29 gene, UL30 gene, UL42 gene, and UL52 gene. In certain embodiments, the early gene is a UL29 gene.

“Late gene” or “L gene” or “γ gene” refers to genes that are required for DNA replication for maximal expression. Late genes mainly encode structural proteins, and start to be transcribed following viral DNA replication. The expression of late genes ultimately leads to the assembly and release of infectious particles (Gruffat, Frontiers in Microbiology (2016); 7:869). Non-limiting examples of late genes of HSV-1 include UL1 gene, UL6 gene, UL15 gene, UL16 gene, UL18 gene, UL19 gene, UL22 gene, UL26 gene, UL26.5 gene, UL27 gene, UL28 gene, UL31 gene, UL32 gene, UL33 gene, UL34 gene, UL35 gene, UL36 gene, UL37 gene, UL38 gene, UL48 gene, UL49.5 gene, and US6 gene. In certain embodiments, the late genes are selected from the group consisting of a UL6 gene, a UL15 gene, a UL19 gene, a UL22 gene, a UL32 gene, a UL33 gene, a UL37 gene, and a UL48 gene.

RL2 encodes ICP0, a 775 amino acid protein that is a transactivator of gene expression. The RL2 gene is one of five immediate early genes expressed by herpes viruses. ICP0 is involved in activating the expression of delayed early and late genes (Lees-Miller et al. 1996, Journal of Virology 70(11): 7471-7477). ICP0 is thought to be involved in neurovirulence. In cell culture, ICP0 has been found to be required for reactivation from latency (Leib et al. 1989, Journal of Virology 63:759-768). Deletion mutants not expressing RL2 have been shown to be unable to replicate in vitro (Sacks and Schaffer 1987, Journal of Virology 61(3):829-839). In certain embodiments, knockout of RL2 can disable the ability of HSV-1 and/or HSV-2 to reactivate from latency. In certain embodiments, knockout of RL2 can disable the ability of HSV-1 and/or HSV-2 to replicate. In certain embodiments, knockout of RL2 can disable the ability of HSV-1 and/or HSV-2 to infect and/or establish latent infections in neural tissue.

RS1 plays an important role in the expression of the immediate early genes by HSV-1 and HSV-2. RS1 is one of five immediate early genes expressed by herpes viruses and is a major transcriptional regulator. RS1 encodes the viral protein ICP4. ICP4 is important for controlling the overall expression of both early and late genes produced by HSV-1 and HSV-2. The RS1 gene is similar in HSV-1 and HSV-2

UL54 encodes ICP27, a highly conserved, multi-functional protein. ICP27 is involved in transcription, RNA processing, RNA export and translation (Sandri-Goldin, Frontiers in Bioscience 2008; 13:5241-5256). ICP27 also shuts off host gene expression during HSV-1 infection. Knockout of UL54 can disable HSV-1 transcription, translation and RNA processing and therefore prevent and/or cure HSV-1 infection.

US1 gene encodes ICP22, that is required for the activation of cdc2 and degradation of the cyclin A and B. ICP22 and the UL13 protein kinase act together to mediate the degradation of cyclin B. Knocking out ICP22 and UL13 results in the accumulation of a set of late proteins. (See Knipe et al., “Chapter 60: Herpes Simplex Viruses,” FIELD VIROLOGY, Lippincott Williams & Wilkins, 6^{th} ed. (2013)).

US1.5 gene encodes a nonessential protein, and the α22 methionine 171 codon is the initiator codon of the of the US1.5 ORF. Some known functions of ICP22 map to the US1.5 ORF. (See Knipe).

US12 gene is also called α47 gene, and encodes ICP47. ICP47 can block the translocation of antigenic peptides into the ER, and thus prohibits the presentation of those peptides at the cell surface. (See Knipe).

UL29 encodes for ICP8, and plays several crucial roles in viral infection. ICP8 participates in the opening of the viral DNA origin to initiate replication by interacting with the origin-binding protein, and can disrupt loops, hairpins and other secondary structures present on ssDNA to reduce and eliminate pausing of viral DNA polymerase at specific sites during elongation. ICP8 also promotes viral DNA recombination by performing strand-transfer, characterized by the ability to transfer a DNA strand from a linear duplex to a complementary single-stranded DNA circle. ICP8 can also catalyze the renaturation of complementary single strands. Additionally, ICP8 reorganizes the host cell nucleus, leading to the formation of pre-replicative sites and replication compartments. This process is driven by the protein which can form double-helical filaments in the absence of DNA.

UL5 encodes a protein that is part of the heterotrimeric helicase-primase complex. The function of UL5 is dependent on its interactions with ICP8. (See Knipe).

UL8 encodes a subunit of the putative primase that is part of the heterotrimeric helicase-primase complex. UL8 is required for the unwinding of DNA coated by ICP8. (See Knipe).

UL9 encodes a viral origin binding protein. UL9 is required for the initiation of DNA synthesis. Degradation of UL9 enables a rolling cycle type of DNA replication. (See Knipe).

UL23 encodes a wide-spectrum nucleoside kinase TK. TK can phosphorylate purine and pyrimidine nucleosides and their analogs. TK plays a role in HSV infections treatment. (See Knipe).

UL30 encodes a protein that is the catalytic subunit of the viral DNA polymerase. (See Knipe).

UL42 encodes a DNA polymerase processivity factor that is an accessary protein of DNA polymerase. UL42 is required for viral replication. (See Knipe).

UL52 encodes a subunit of the HSV helicase/primase complex that has strong affinity for ICP8. ICP8 and the helicase/primase complex (UL8/UL5/UL52) form a nuclear complex in transfected cells. (See Knipe).

UL6 encodes a protein that forms a portal in the viral capsid through which viral DNA is translocated during DNA packaging. The UL6 protein assembles as a dodecamer at a single fivefold axe of the T=16 icosahedric capsid, and binds to the molecular motor that translocates the viral DNA, termed terminase (White et al., Journal of Virology 2003; 77(11):6351-8).

The UL15 protein of HSV-1 plays a key role a key role in localizing the terminase complex to DNA replication compartments, and that it can interact independently with UL28 and UL33 (Higgs et al., Journal of General Virology 2008; 89(7):1709-15). The UL15 protein, together with UL28 and UL33 proteins, form a terminase complex responsible for cleavage and packaging of the viral genome into pre-assembled capsids.

UL19 (also known as VP5) encodes the HSV-1 major capsid protein, VPS. Proper assembly of the viral capsid is known to be an essential part of viral replication, assembly, maturation and infection (Homa et al., Reviews of Medical Virology 1997; 7(2):107-122). RNAi-mediated knockdown of VP5 along with another capsid protein, VP23, in vitro, greatly diminished HSV-1 proliferation (Jin et al., PLoS One 2014; 9(5): e96623). Knockout of UL19 can disable HSV-1 proliferation and therefore prevent, treat or cure HSV-1 infection.

UL22 encodes glycoprotein H (gH), which is one of the four glycoproteins essential for virus entry. The other three glycoproteins are gD, gL, and gB. A coordinated interaction among multiple viral glycoproteins is required to mediate fusion of the viral envelope with the cell membrane. gD binds to a cellular receptor activates a gH/gL heterodimer, and this step subsequently triggers gB, the conserved herpesvirus fusion protein, to mediate virus-cell or cell-cell membrane fusion (Fan et al., Journal of Virology 2015; 89(14):7159-69).

UL32 encodes packaging protein UL32, and plays a role in efficient localization of neo-synthesized capsids to nuclear replication compartments, thereby controlling cleavage and packaging of virus genomic DNA. Additionally, the UL32 protein plays a role in cleavage and/or packaging of viral DNA and in maturation and/or translocation of viral glycoproteins to the plasma membrane (Chang et al., Journal of Virology 1996; 70(6):3938-46.)

UL33 encodes a 130 amino acid protein that is essential for the cleavage of concatemeric viral DNA into monomeric genomes and their packaging into preformed capsids. UL33 protein, together with UL15 and UL28 proteins form a protein complex called terminase. During HSV-1 infection, empty procapsids are assembled and subsequently filled with the viral genome by means of a terminase (Heming et al., Journal of Virology 2014; 88(1)225-236).

UL37 encodes a 120-kDa phosphorylated polypeptide that resides in the tegument structure of the virion and is important for morphogenesis. The UL37 polypeptide is expressed late in the virus replication cycle and is a component of both mature virions and light particles. During HSV-1 infection, the UL37 polypeptide is distributed throughout the infected cell but is predominantly localized to the cytoplasm (Desai et al., Journal of Virology 2008; 82(22): 11354-11361.

UL48 encodes the viral protein known as VP16 in HSV-1. VP-16 has been shown to be important in viral egress, the process by which the assembled viral capsid leaves the host nucleus and enters the cytoplasm (Mossman et al., Journal of Virology 2000; 74(14): 6287-6289). Mutation of UL48 in cell culture decreased the ability of HSV-1 to assemble efficiently (Svobodova et al., Journal of Virology 2012; 86(1): 473-483). Knockout of UL48 can disable HSV-1 assembly and egress and therefore prevent and/or cure HSV-1 infection.

UL1 encodes glycoprotein L that regulates the fusogenic activity of glycoprotein gH encoded by UL22. (See Knipe).

UL16 encodes a tegument protein that forms complexes with UL11, gE, VP22, and UL2. UL16 is essential for virus replication because it helps the DNA DNA-containing capsids to exit the nucleus of infected cells. (Gao et al., Journal of Virology 2017; 91(10): e00350-17).

UL18 encodes a capsid protein VP23. Inhibiting the expression of VP23 and/or VP5 affects the replication of HSV-1. (Jin et al., PLoS ONE 2014; 9(5): e96623).

UL26 encodes a capsid scaffolding protein that plays a role in capsid assembly. It acts as a scaffold protein by binding major capsid protein in the cytoplasm, and inducing the nuclear localization of both proteins. (See Knipe).

UL26.5 encodes a protein that is the same as the C-terminal sequence of the UL26 protein. (See Knipe).

UL27 encodes an envelope glycoprotein B (gB) that is essential for the fusion of the viral envelop with the host cell plasma membrane. (See Knipe).

UL28 encodes a protein that is a subunit of a tripartite terminase. UL28 interacts with UL15 and UL6, and is essential for cleavage of replicated concatemeric viral DNA. (White et al., Journal of Virology (2003); 77(11):6351-8).

UL31 encodes a catalytic subunit of the viral DNA polymerase. (See Knipe).

UL34 encodes a type II membrane protein that is required for efficient envelopment of progeny virions at the nuclear envelope. (Reynolds et al., Journal of Virology (2001); 75(18):8803-8817).

UL35 encodes VP26 that is a capsid protein. VP26 forms a hexameric structure that is located on the outer surface of each hexon. (See Knipe).

UL36 encodes VP1-2 that is a tegument protein. VP1-2 is required for the exit of virions from the cytoplasm of the host cell. (See Knipe).

UL38 encodes VP19C that is a capsid protein. VP19C interacts with VP23, forming a triplex connecting adjacent hexons and pentons. (See Knipe).

UL49.5 encodes a gN membrane-associated protein that is enriched in virions. UL49.5 protein interacts with gM. (See Knipe).

UL6 encodes a portal protein that interacts with UL15 and UL28. All three proteins are essential for cleavage of replicated concatemeric viral DNA (White et al., Journal of Virology 2003; 77(11):6351-8).

In certain embodiments, inhibiting essential viral functions, e.g., viral gene transcription, viral genome replication and viral capsid formation, decreases the duration of recurrent infection and/or decrease shedding of viral particles. In certain embodiments, subjects also experience shorter duration(s) of illness, decreased risk of transmission to sexual partners, decreased risk of transmission to the fetus in the case of pregnancy and/or the potential for full clearance of HSV-1 (cure).

Knockout of one or more copies (e.g., 1, 2, 3, 4, 5, 6, 7, 8, 9 or 10 copies) of one or more HSV-1 genes can be performed prior to disease onset or after disease onset (including early in the disease course).

In certain embodiments, the method disclosed herein comprises a prophylactic treatment of a virus infection in a subject. In certain embodiments, the method disclosed herein comprises initiating treatment of a subject prior to the subject is exposed to a virus. In certain embodiments, the method disclosed herein comprises initiating treatment of a subject who is at risk of being exposed to the virus. In certain embodiments, the method disclosed herein comprises initiating treatment of a subject at risk of developing a virus infection or a virus-infection related disease. Subjects at risk of developing a virus infection or a virus-infection related disease include but not limited to healthcare workers, immune deficient patients, children, elders, and pregnant women.

In certain embodiments, the method disclosed herein comprises initiating treatment of a subject prior to disease onset. In certain embodiments, the method comprises initiating treatment of a subject after disease onset. In certain embodiments, the method comprises initiating treatment of a subject well after disease onset, e.g., 1, 2, 3, 4, 5, 6, 7, 8, 9, 10, 12, 16, 24, 36, 48 or more months after onset of HSV-1 infection. In certain embodiments, the method comprises initiating treatment of a subject well after disease onset, e.g., 1, 2, 3, 4, 5, 10, 15, 20, 25, 40, 50 or 60 years after onset of HSV-1 infection. This can be effective as disease progression is slow in some cases and a subject can present well into the course of illness.

In certain embodiments, the method comprises initiating treatment of a subject in an advanced stage of disease, e.g., during latent periods. In certain embodiments, the method comprises initiating treatment of a subject in the case of severe, acute disease affecting eyes. Overall, initiation of treatment for subjects at all stages of disease is expected to improve healing, decrease duration of disease and be of benefit to subjects.

In certain embodiments, the method comprises initiating treatment of a subject prior to disease expression. In certain embodiments, the method comprises initiating treatment of a subject in an early stage of disease, e.g., when a subject has been exposed to HSV-1 or is thought to have been exposed to HSV-1. In certain embodiments, the method comprises initiating treatment of a subject prior to disease expression. In certain embodiments, the method comprises initiating treatment of a subject in an early stage of disease, e.g., when a subject has been tested positive for HSV-1 infections but has no signs or symptoms.

In certain embodiments, the method comprises initiating treatment at the appearance of any of the following symptoms consistent or associated with optic HSV: pain, photophobia, blurred vision, tearing, redness/injection, loss of vision, floaters, or flashes. In certain embodiments, the method comprises initiating treatment at the appearance of any of the following findings on ophthalmologic exam consistent or associated with optic HSV, also known as HSV-related keratitis: small, raised clear vesicles on corneal epithelium; irregular corneal surface, punctate epithelial erosions; dense stromal infiltrate; ulceration; necrosis; focal, multifocal, or diffuse cellular infiltrates; immune rings; neovascularization; or ghost vessels at any level of the cornea.

In certain embodiments, the method comprises initiating treatment at the appearance of any of the following findings on ophthalmologic exam consistent or associated with HSV-1 retinitis or acute retinal necrosis: reduced visual acuity; uveitis; vitritis; scleral injection; inflammation of the anterior and/or vitreous chamber/s; vitreous haze; optic nerve edema; peripheral retinal whitening; retinal tear; retinal detachment; retinal necrosis; evidence of occlusive vasculopathy with arterial involvement, including arteriolar sheathing and arteriolar attenuation.

In certain embodiments, the method comprises initiating treatment prior to organ transplantation or immediately following organ transplantation.

In certain embodiments, the method comprises initiating treatment in case of suspected exposure to HSV-1.

In certain embodiments, the method comprises initiating treatment of a subject who suffers from or is at risk of developing severe manifestations of HSV-1 infections, e.g., neonates, subjects with HIV, subjects who are on immunosuppressant therapy following organ transplantation, subjects who have cancer, subjects who are undergoing chemotherapy, subjects who will undergo chemotherapy, subjects who are undergoing radiation therapy, subjects who will undergo radiation therapy.

Both HIV positive subjects and post-transplant subjects can experience severe HSV-1 activation or reactivation, including HSV-encephalitis and meningitis, due to immunodeficiency. Neonates are also at risk for severe HSV-encephalitis due to maternal-fetal transmission during childbirth. Inhibiting essential viral functions, e.g., viral gene transcription, viral genome replication and viral capsid formation, can provide superior protection to said populations at risk for severe HSV-1 infections. Subjects can experience lower rates of HSV-1 encephalitis and/or lower rates of severe neurologic sequelae following HSV-1 encephalitis, which will profoundly improve quality of life.

In certain embodiments, the method comprises initiating treatment in a subject who has been tested positive for HSV-1 infection via viral culture, direct fluorescent antibody study, skin biopsy, PCR, blood serologic test, CSF serologic test, CSF PCR, or brain biopsy.

In certain embodiments, the method comprises initiating treatment in any subject who has been exposed to HSV-1 and at high risk for severe sequelae from HSV infection.

In certain embodiments, a cell is manipulated by editing (e.g., introducing a mutation in) one or more target HSV-1 genes. In certain embodiments, the expression of one or more target genes are modulated, e.g., in vivo.

In certain embodiments, the method comprises delivery of gRNA by an AAV. Non-limiting exemplary AAV vectors include serotype 1, 2, 3, 4, 5, 6, 7, 8 or 9 vector. In certain embodiments, the method comprises delivery of gRNA by a lentivirus. In certain embodiments, the method comprises delivery of gRNA by a nanoparticle. In certain embodiments, the method comprises delivery of gRNA by a gel-based AAV for topical therapy.

In certain embodiments, the method further comprising treating the subject a second antiviral therapy, e.g., an anti-HSV-1 therapy described herein. The systems and compositions described herein can be administered concurrently with, prior to, or subsequent to, one or more additional therapies or therapeutic agents. The systems, composition and the other therapy or therapeutic agent can be administered in any order. In certain embodiments, the effect of the two treatments is synergistic. Exemplary anti-HSV-1 therapies include, but are not limited to, acyclovir, valacyclovir, famciclovir, penciclovir, or a vaccine.

### Genome Editing Systems

Various genome editing systems known in the art can be used for the methods disclosed herein. Non-limiting examples of genome editing systems that can be used with the presently disclosed subject matter include, but are not limited to CRISPR systems, zinc-finger nuclease (ZFN) systems, transcription activator-like effector nuclease (TALEN) systems, meganuclease (MN) systems, MegaTAL systems, other targeted endonuclease systems, and other chimeric endonuclease systems.

In certain embodiments, the genome editing system has RNA-guided DNA editing activity. In certain embodiments, the genome editing system includes at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Naturally occurring CRISPR systems are organized evolutionarily into two classes and five types (Makarova et al. Nat Rev Microbiol. 2011 June; 9(6): 467-477 (Makarova), incorporated by reference herein), and while genome editing systems of the present disclosure can adapt components of any type or class of naturally occurring CRISPR system, the embodiments presented herein are generally adapted from Class 2, and type II or V CRISPR systems. Class 2 systems, which encompass types II and V, are characterized by relatively large, multidomain RNA-guided nuclease proteins (e.g., Cas9 or Cpf1) and one or more guide RNAs (e.g., a crRNA and, optionally, a tracrRNA) that form ribonucleoprotein (RNP) complexes that associate with (i.e. target) and cleave specific loci complementary to a targeting (or spacer) sequence of the crRNA. Genome editing systems according to the present disclosure similarly target and edit cellular DNA sequences, but differ significantly from CRISPR systems occurring in nature. For example, the unimolecular guide RNAs described herein do not occur in nature, and both guide RNAs and RNA-guided nucleases according to this disclosure can incorporate any number of non-naturally occurring modifications.

The genome editing system disclosed herein can be delivered into subjects or cells using a retroviral vector, e.g., gamma-retroviral vectors, and lentiviral vectors. Combinations of retroviral vector and an appropriate packaging line are suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art. Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for gene editing of the mammalian cell disclosed herein. For example, a nucleic acid molecule can be introduced into the cells/subjects by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of nucleic acid molecules into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

Genome editing systems can be implemented (e.g. administered or delivered to a cell or a subject) in a variety of ways, and different implementations can be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as a lipid or polymer micro- or nano-particle, micelle, liposome, etc. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus; and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or can be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (Maeder), which is incorporated by reference herein, describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e. flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino, et al. (“Cotta-Ramusino”), incorporated by reference herein, describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al. (“Palestrant”, incorporated by reference herein) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

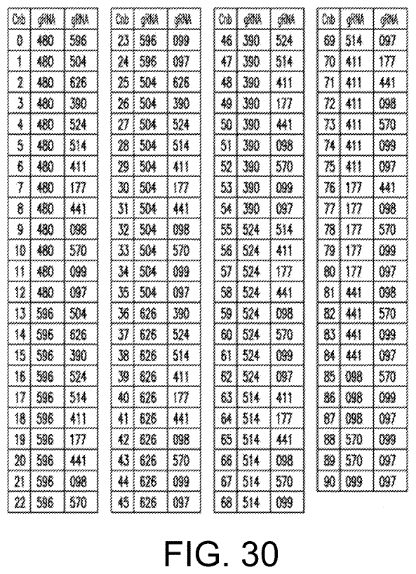

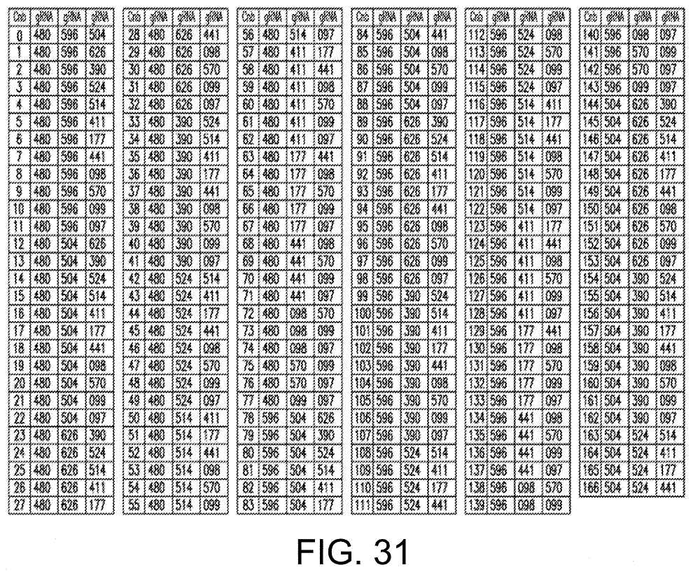

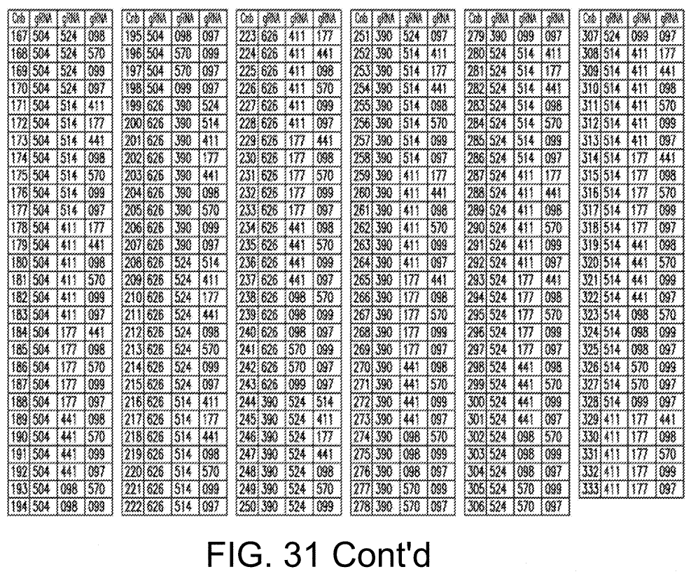

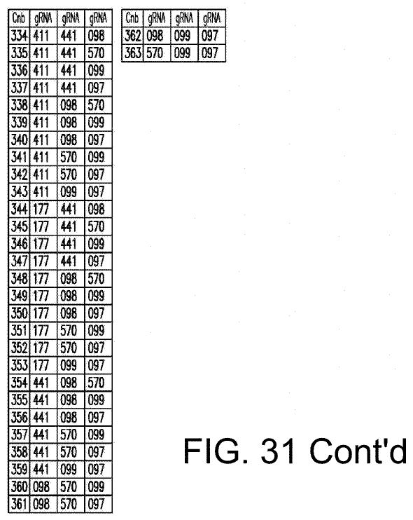

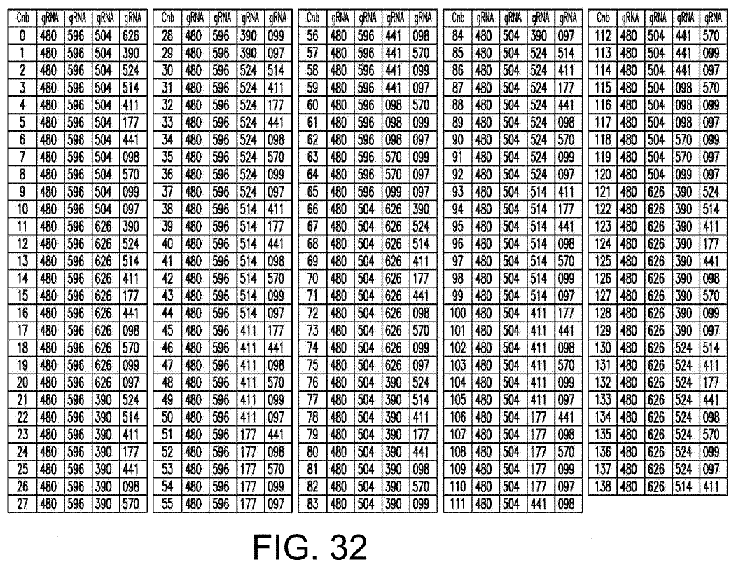

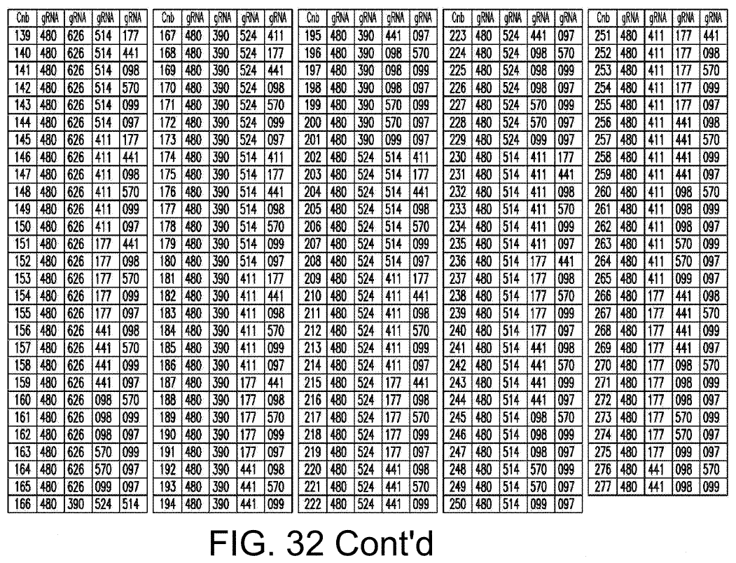

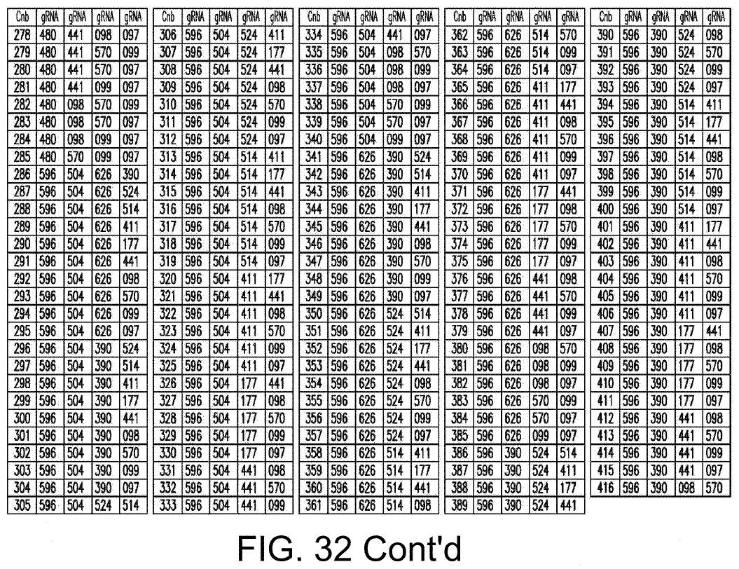

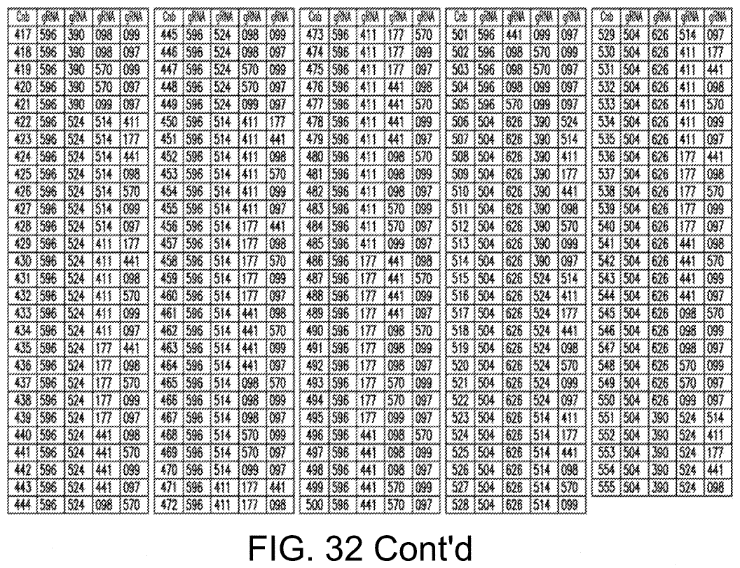

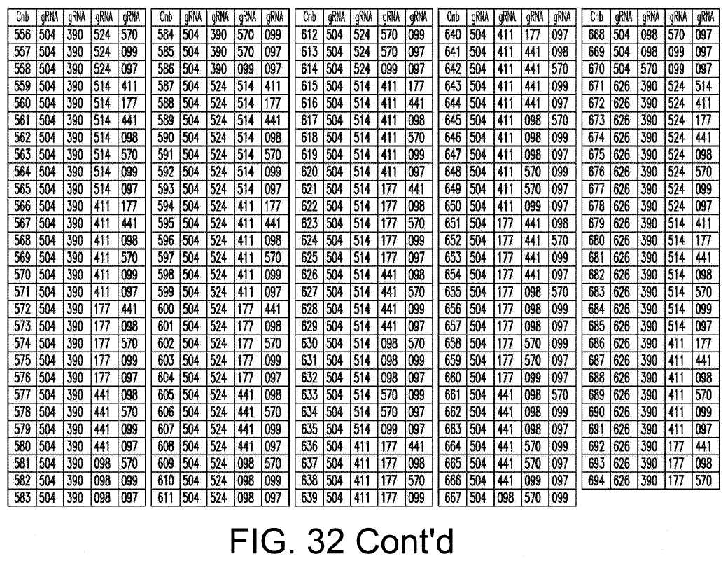

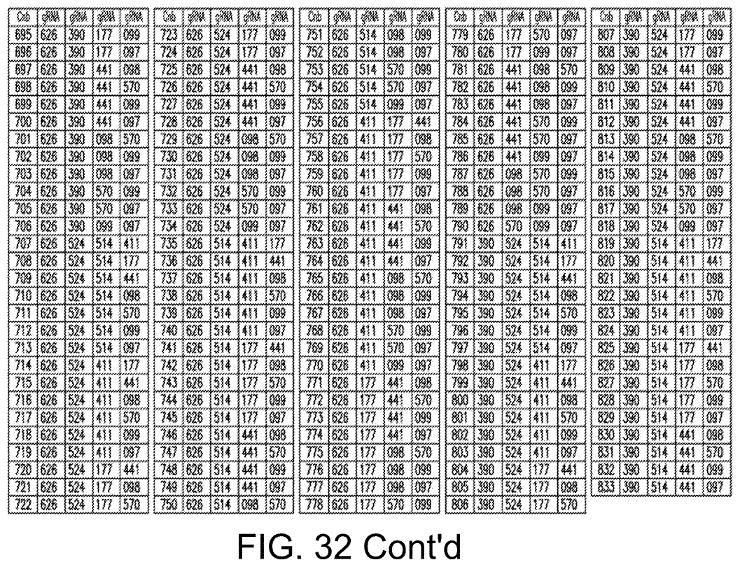

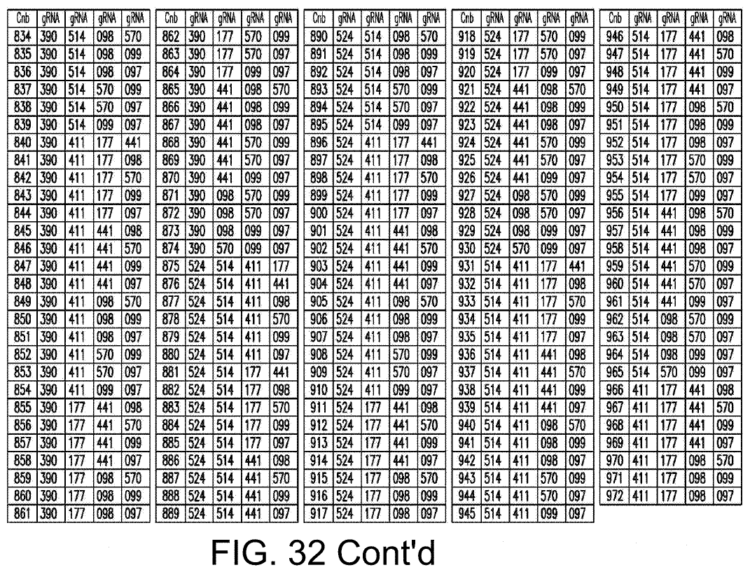

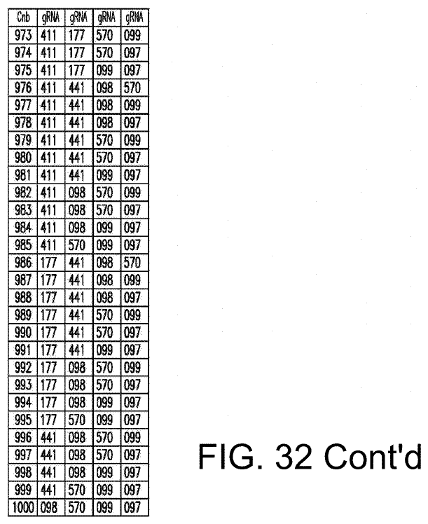

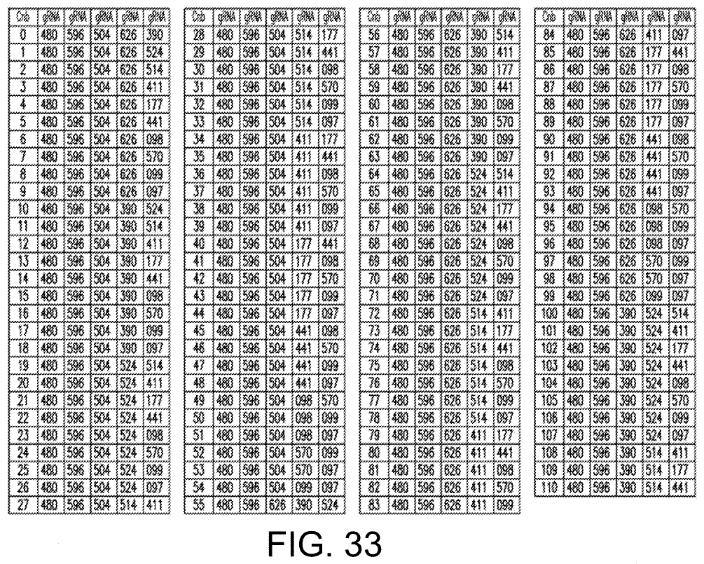

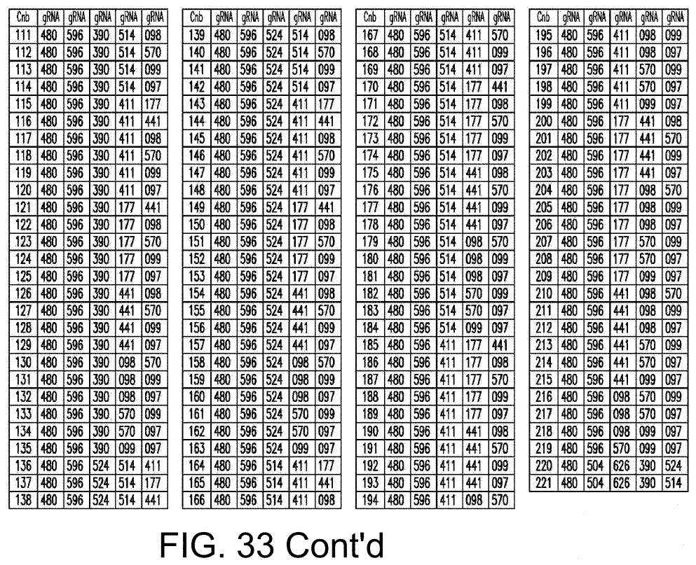

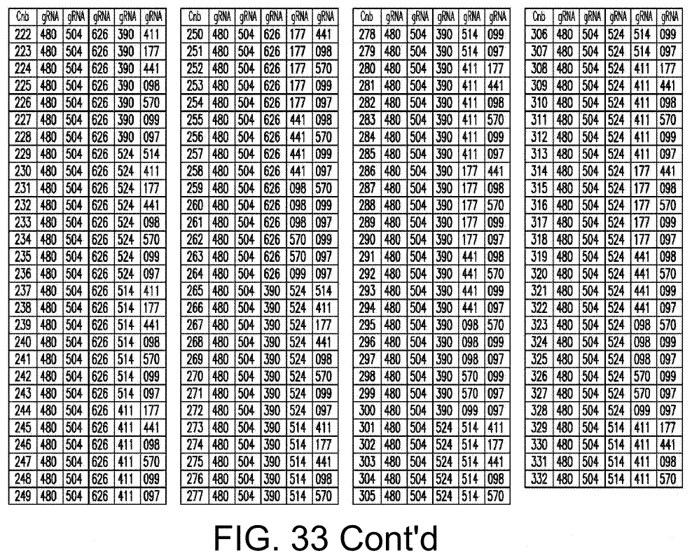

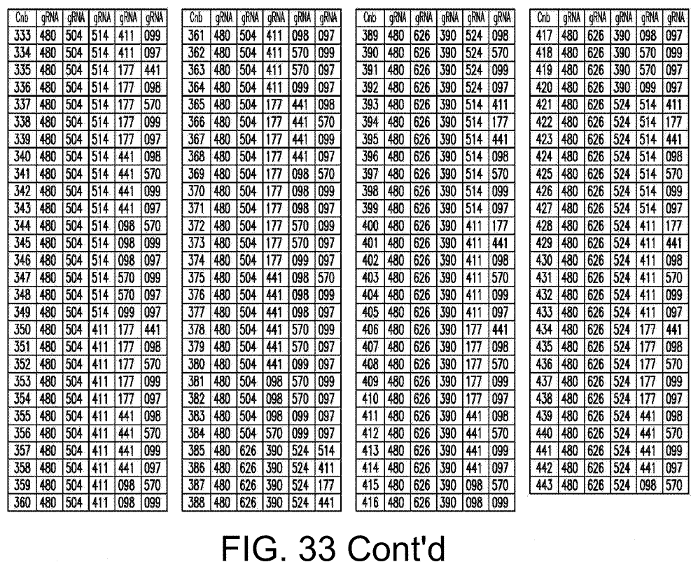

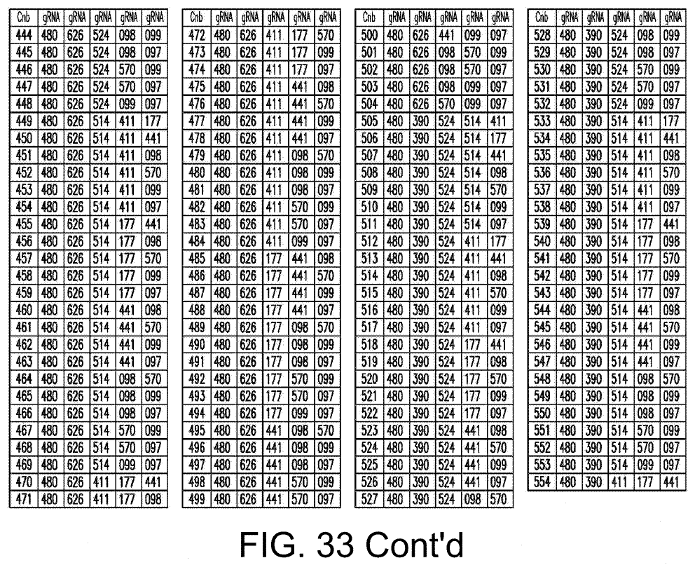

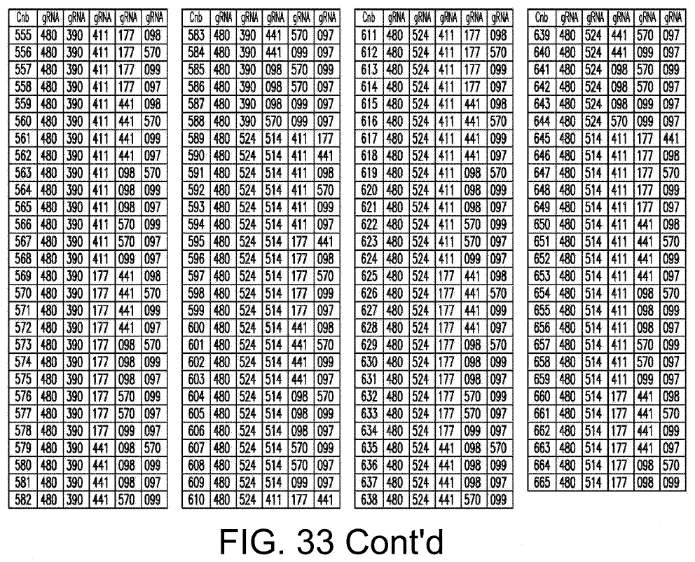

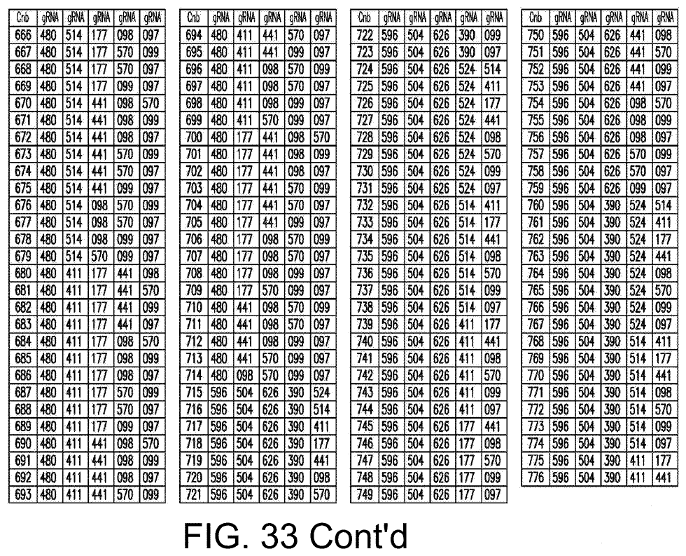

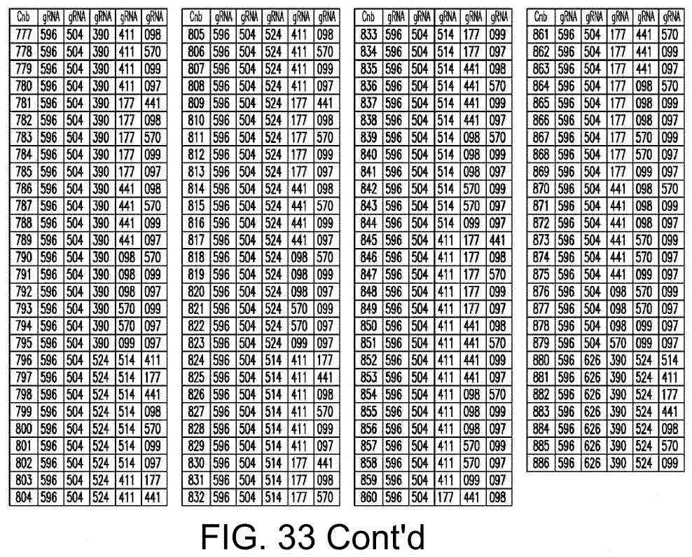

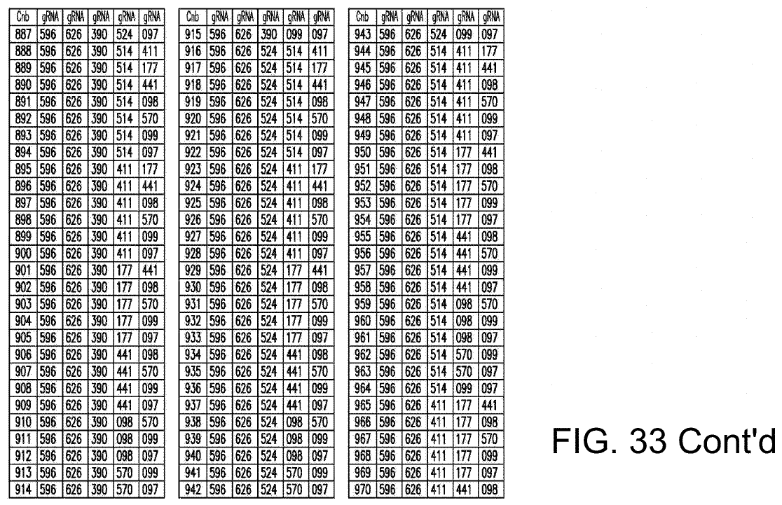

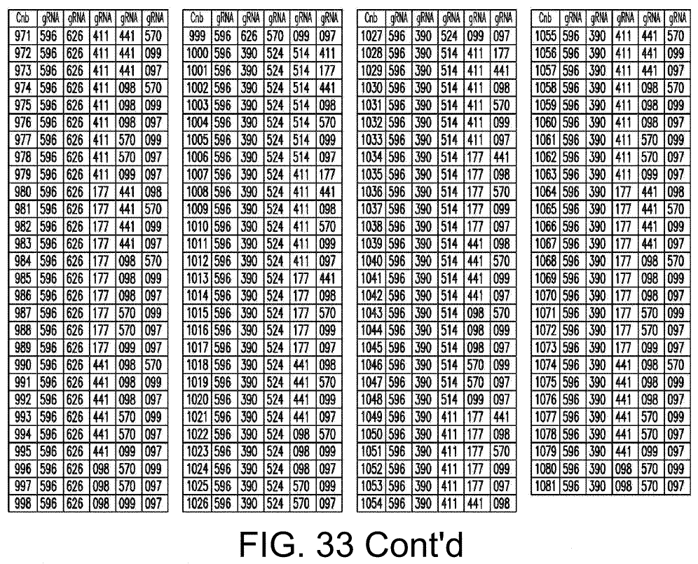

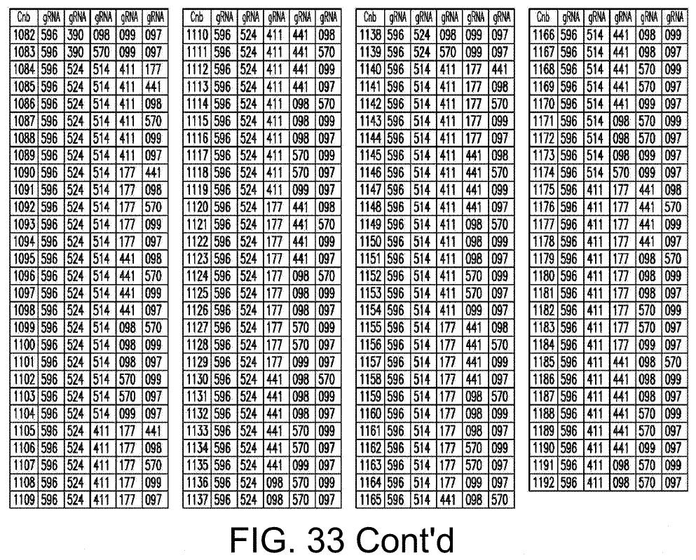

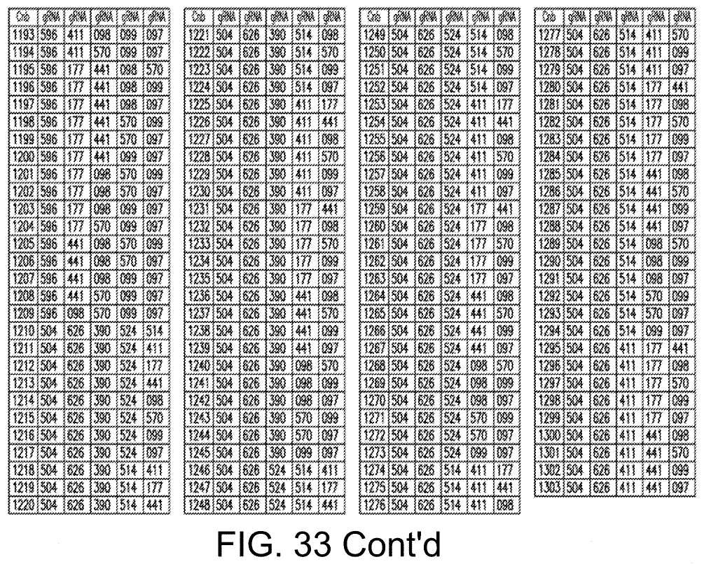

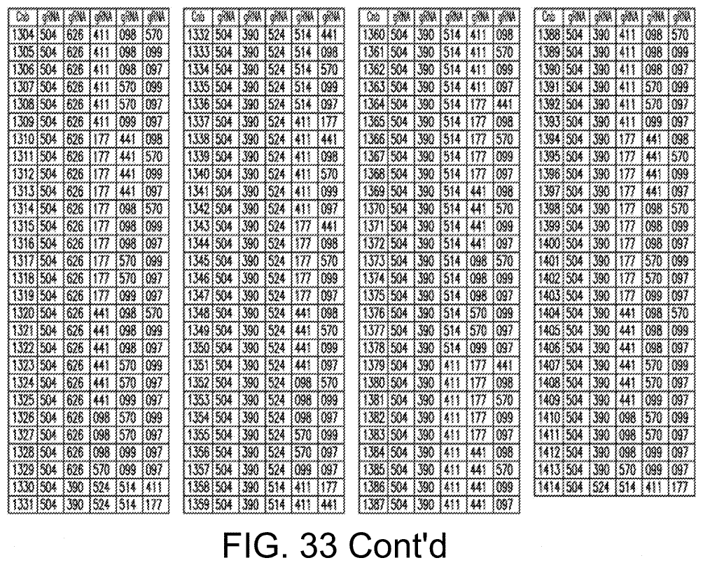

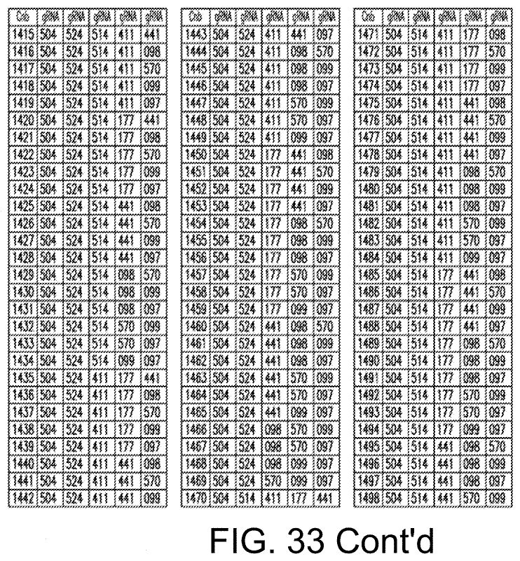

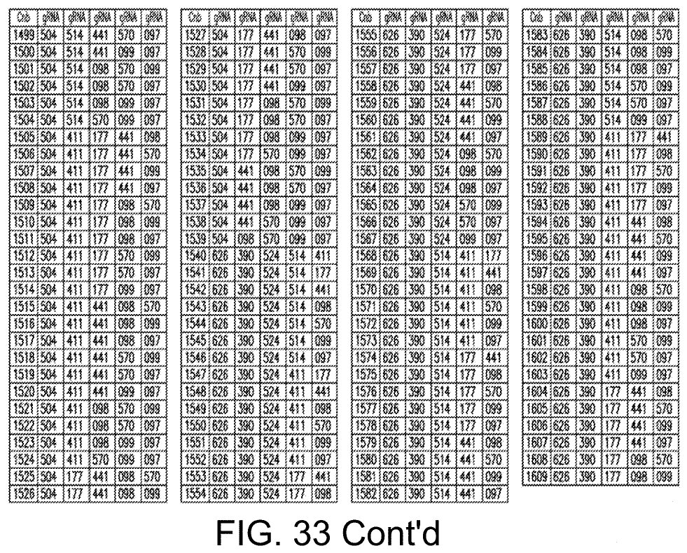

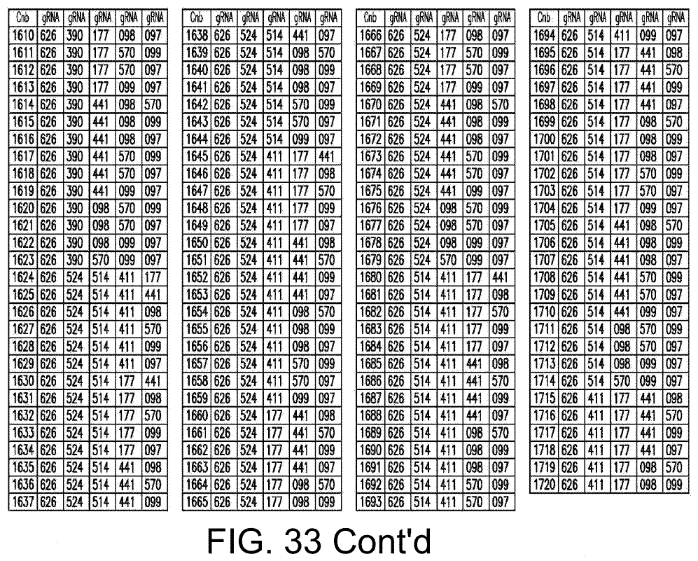

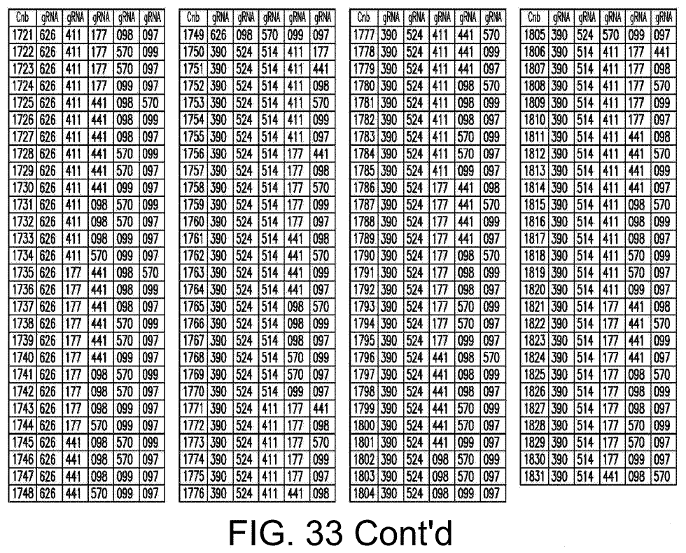

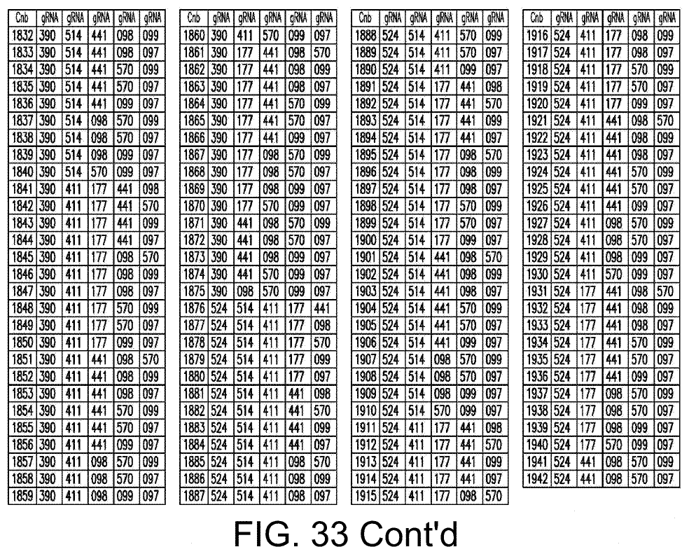

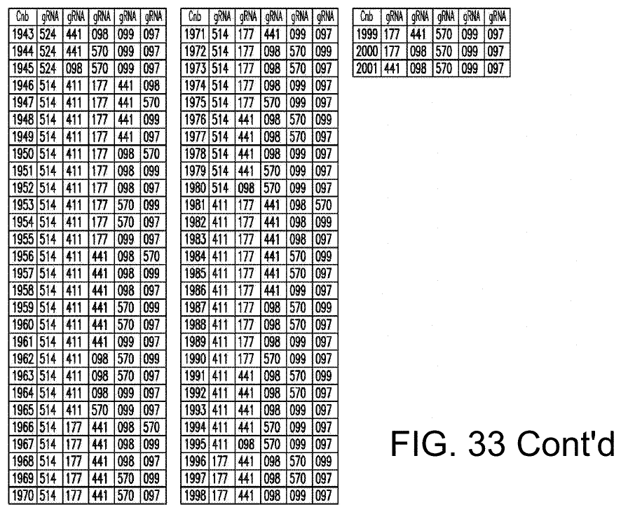

In various non-limiting embodiments, the genome editing systems of the present disclosure target two specific nucleotide sequences through the use of a combination of two gRNAs. FIG. 30 lists non-limiting exemplary combinations of two gRNAs of the present disclosure. In various non-limiting embodiments, the genome editing systems of the present disclosure target three specific nucleotide sequences through the use of a combination of three gRNAs. FIG. 31 lists non-limiting exemplary combinations of three gRNAs of the present disclosure. In various non-limiting embodiments, the genome editing systems of the present disclosure target four specific nucleotide sequences through the use of a combination of four gRNAs. FIG. 32 lists non-limiting exemplary combinations of four gRNAs of the present disclosure. In various non-limiting embodiments, the genome editing systems of the present disclosure target five specific nucleotide sequences through the use of a combination of five gRNAs. FIG. 33 lists non-limiting exemplary combinations of five gRNAs of the present disclosure.

Genome editing systems can, in some instances, form double strand breaks that are repaired by cellular DNA double-strand break mechanisms such as NHEJ or HDR. These mechanisms are described throughout the literature, for example by Davis & Maizels, PNAS, 111(10):E924-932, Mar. 11, 2014 (Davis) (describing Alt-HDR); Frit et al. DNA Repair 17(2014) 81-97 (Frit) (describing Alt-NHEJ); and Iyama and Wilson III, DNA Repair (Amst.) 2013-August; 12(8): 620-636 (Iyama) (describing canonical HDR and NHEJ pathways generally).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing single- or double-strand breaks. For example, a genome editing system can include an RNA-guided nuclease fused to a functional domain that acts on DNA, thereby modifying the target sequence or its expression. As one example, an RNA-guided nuclease can be connected to (e.g. fused to) a cytidine deaminase functional domain, and can operate by generating targeted C-to-A substitutions. Exemplary nuclease/deaminase fusions are described in Komor et al. Nature 533, 420-424 (19 May 2016) (“Komor”), which is incorporated by reference. Alternatively, a genome editing system can utilize a cleavage-inactivated (i.e. a “dead”) nuclease, such as a dead Cas9 (dCas9), and can operate by forming stable complexes on one or more targeted regions of cellular DNA, thereby interfering with functions involving the targeted region(s) including, without limitation, mRNA transcription, chromatin remodeling, etc.

In certain embodiments, the genome editing system or composition comprises a first gRNA molecule comprising a first targeting domain that is complementary with a first target sequence of a first HSV-1 gene and a second gRNA molecule comprising a second targeting domain that is complementary with a second target sequence of a second HSV-1 gene. The first and second HSV-1 genes can be independently selected from immediate early HSV-1 genes (e.g., those disclosed herein), early HSV-1 genes (e.g., those disclosed herein), and late HSV-1 genes (e.g., those disclosed herein). In certain embodiments, at least one of the first and second HSV-1 genes is a late HSV-1 gene. For example, the first HSV-1 gene is a late HSV-1 gene, and the second HSV-1 gene is an immediate early HSV-1 gene, an early HSV-1 gene, or a late HSV-1 gene. In certain embodiments, the first HSV-1 gene is a late HSV-1 gene, and the second HSV-1 gene is an immediate early HSV-1 gene. In certain embodiments, the first HSV-1 gene is a UL48 gene, and the second HSV-1 gene is a RL2 gene.

In certain embodiments, the expression of at least one component of a genome editing system disclosed herein (e.g., at least one gRNA molecule, or at least one RNA-guided nuclease) is regulated by a promoter derived from a genome of HSV-1, and/or HSV-2. The promoters disclosed herein have the advantages of 1) being resistant to HSV-1 dependent cellular gene silencing during reactivation, 2) has differential expression at latency and reactivation of HSV-1 (e.g., weak expression at latency and strong expression at reactivation, and/or 3) only has activity in target tissues (e.g., latency tissues and cells, e.g., trigeminal dorsal root ganglion, the cervical dorsal root ganglia, and the sacral dorsal root ganglia).

In certain embodiments, the promoters disclosed herein are activated by HSV-1 gene expression, and the activated promoter in turn induces the expression of one or more components of the gene editing system. As a result, the induced components of the gene editing system are expressed when the HSV-1 genes are expressed. For example, upon introduction of a gene editing system into a cell infected by HSV-1, expression of the gene editing system is modulated by the transcriptional activity of HSV-1 via activation of the promoter.

In certain embodiments, the promoter is operably linked to a polynucleotide encoding at least one gRNA molecule, and/or a polynucleotide encoding an RNA-guided nuclease.

In certain embodiments, the component(s) of the genome editing system exhibiting induction of expression in response to viral promoter activation has a low expression level during HSV-1 latency, e.g., an expression level lower than the expression level during HSV-1 reactivation. In certain embodiments, the component(s) of the genome editing system exhibiting induction of expression in response to viral promoter activation has a high expression level during HSV-1 reactivation. In certain embodiments, the expression level of the component(s) of the genome editing system exhibiting induction of expression in response to viral promoter activation during HSV-1 latency is at least about 10%, at least about 20%, at least about 30%, at least about 40% or at least about 50% lower than the expression level of the component(s) of the genome editing system exhibiting induction of expression in response to viral promoter activation during HSV-1 reactivation.

In certain embodiments, the promoter disclosed herein is derived from a gene (e.g., a DNA sequence encoding and directing the expression of a protein) of the Herpesviridae family. In certain embodiments, the promoter is derived from a gene of a virus of Alphaherpesvirinae subfamily, Betaherpesvirinae subfamily, or Gammaherpesvirinae subfamily. In certain embodiments, the promoter is derived from a gene of a Iltovirus, a Mardivirus, a Simplexvirus, a Scutavirus, or a Varicellovirus. In certain embodiments, the promoter is derived from a gene of an Cytomegalovirus (CMV), a Morumegalovirus, a Proboscivirus, or a Roseolovirus. In certain embodiments, the promoter is derived from a gene of a Lymphocryptovirus, a Macavirus, a Percavirus, or a Rhadinovirus. In certain embodiments, the promoter is derived from a gene of a Cytomegalovirus (HCMV), a Kaposi Sarcoma-Associated Herpesvirus (KSHV), an Epstein-Barr virus (EBV), or a Varicella-Zoster virus (VZV). In certain embodiments, the promoter is derived from a gene of a Human Immunodeficiency Virus (HIV) or a Human Papillomavirus (HPV). In certain embodiments, the promoter is derived from a HSV gene. In certain embodiments, the promoter is derived from a HSV-2 gene. In certain embodiments, the promoter is derived from an HSV-1 gene. In certain embodiments, the gene is an immediate-early gene, a late gene, or an early gene of HSV-1. In certain embodiments, the gene is selected from the group consisting of LAT, RL2, US12, S1, UL54, UL23, UL29, UL39, US6, UL19, UL37, UL27, UL44, and UL38. Non-limiting exemplary promoters that can be used with the present disclosure include SEQ ID NOs: 412-425 as follows:

LAT promoter
[SEQ ID NO.: 412]
CTGCAGACAGGGGCACCGCGCCCGGAAATCCATTAGGCCGCAGACGAGG
AAAATAAAATTACATCACCTACCCACGTGGTGCTGTGGCCTGTTTTTGC
TGCGTCATCTCAGCCTTTATAAAAGCGGGGGCGCGGCCGTGCCGATCGC
GGGTGGTGCGAAAGACTTTCCGGGCGCGTCCGGGTGCCGCGGCTCTCCG
GGCCCCCCTGCAG
RL2 promoter
[SEQ ID NO.: 413]
CCCGGGAGCTCCGCACCAAGCCGCTCTCCGGAGAGACGATGGCAGGAGC
CGCGCATATATACGCTTGGAGCCAGCCCGCCCTCACAGGGCGGGCCCGC
CTCGGGGGCGGGACTGGCCAATCGGCGGCCGCCAGCGCGGCGGGGCCCG
GCCAACCAGCGTCCGCCGAGTCTTCGGGGCCCGGCCCATTGGGCGGGAG
TTACCGCCCAATGGGCCGGGCCGCCCACTTCCCGGTATGGTAATTAAAA
ACTTGCAAGAGGCCTTGTTCCGCTTCCCGGTATGGTAATTAGAAACTCA
TTAATGGGCGGCCCCGGCCGCCCTTCCCGCTTCCGGCAATTCCCGCGGC
CCTTAATGGGCAACCCCGGTATTCCCCGCCTCCCGCGCCGCGCGTAACC
ACTCCCCTGGGGTTCCGGGTTATGCTAATTGCTTTTTTGGCGGAACACA
CGGCCCCTCGCGCATTGGCCCGCGGGTCGCTCAATGAACCCGCATTGGT
CCCCTGGGGTTCCGGGTATGGTAATGAGTTTCTTCGGGAAGGCGGGAAG
CCCCGGGGCACCGACGCAGGCCAAGCCCCTGTTGCGTCGGCGGGAGGGG
CATGCTAATGGGGTTCTTTGGGGGACACCGGGTTGGGCCCCCAAATCGG
GGGCCGGGCCGTGCATGCTAATGATATTCTTTGGGGGCGCCGGGTTGGT
CCCCGGGGACGGGGCCGCCCCGCGGTGGGCCTGCCTCCCCTGGGACGCG
CGGCCATTGGGGGAATCGTCACTGCCGCCCCTTTGGGGAGGGGAAAGGC
GTGGGGTATAAGTTAGCCCTGGCCCGACAGTCTGGTCGCATTTGCACCT
CGGCACTCGGAGCGAGACGCAGCAGCCAGGCAGACTCGGGCCGCCCCCT
CTCCGCATCACCACAGAAGCCCCGCCTACGTTGCGACCCCCAGGGACCC
TCCGTCCGCGACCCTCCAGCCGCATACGACCCCC
RS1 promoter
[SEQ ID NO.: 414]
CCCGGGCCCCGCCCCCTGCCCGTTCCTCGTTAGCATGCGGAACGGAAGC
GGAAACCGCCGGATCGGGCGGTAATGAGATGCCATGCGGGGCGGGGCGC
GGACCCACCCGCCCTCGCGCCCCGCCCATGGCAGATGGCGCGGATGGGC
GGGGCCGGGGGTTCGACCAACGGGCCGCGGCCACGGGCCCCCGGCGTGC
CGGCGTCGGGGCGGGGTCGTGCATAATGGAATTCCGTTCGGGGTGGGCC
CGCCGGGGGGGCGGGGGGCCGGCGGCCTCCGCTGCTCCTCCTTCCCGCC
GGCCCCTGGGACTATATGAGCCCGAGGACGCCCCGATCGTCCACACGGA
GCGCGGCTGCCGACACGGATCCACGACCCGACGCGGGACCGCCAGAGAC
AGACCGTCAGACGCTCGCCGCGCCGGGACGCCGATACGCGGACGAAGCG
CGGGAGGGGGATCGGCCGTCCCTGTCCTTTTTCCCACCCAAGCATCGAC
CGGTCCGCGCTAGTTCCGCGTCGACGGCGGGGGTCGTCGGGGTCCGTGG
GTCTCGCCCCCTCCCCCCATCGAGAGTCCGTAGGTGACCTACCGTGCTA
CGTCCGCCGTCGCAGCCGTATCCCCGGAGGATCGCCCCGCATCGGCG
UL23 promoter
[SEQ ID NO.: 415]
GGATCCAAATGAGTCTTCGGACCTCGCGGGGGCCGCTTAAGCGGTGGTT
AGGGTTTGTCTGACGCGGGGGGAGGGGGAAGGAACGAAACACTCTCATT
CGGAGGCGGCTCGGGGTTTGGTCTTGGTGGCCACGGGCACGCAGAAGAG
CGCCGCGATCCTCTTAAGCACCCCCCCGCCCTCCGTGGAGGCGGGGGTT
TGGTCGGCGGGTGGTAACTGGCGGGCCGCTGACTCGGGCGGGTCGCGCG
CCCCAGAGTGTGACCTTTTCGGTCTGCTCGCAGACCCCCGGGCGGCGCC
GCCGCGGCGGCGACGGGCTCGCTGGGTCCTAGGCTCAATGGGGACCGTA
TACGTGGACAGGCTCTGGAGCATCCGCACGACTGCGGTGATATTACCGG
AGACCTTCTGCGGGACGAGCCGGGTCACGCGGCTGACGCGGAGCGTCCG
TTGGGCGACAAACACCAGGACGGGGCACAGGTACACTATCTTGTCACCC
GGAGGCGCGAGGGACTGCAGGAGCTTCAGGGAGTGGCGCAGCTGCTTCA
TCCCCGTGGCCCGTTGCTCGCGTTTGCTGGCGGTGTCCCCGGAAGAAAT
ATATTTGCATGTCTTTAGTTCTATGATGACACAAACCCCGCCCAGCGTC
TTGTCATTGGCGAATTCGAACACGCAGATGCAGTCGGGGCGGCGCGGTC
CCAGGTCCACTTCGCATATTAAGGTGACGCGTGTGGCCTCGAATACCGA
GCGACCCTGCAGCGACCCGCTTAACAGCGTCAACAGCGTGCCGCAGATC
TTGGTGGCGTGAAACTCCCGCACCTCTTCGGCCAGCGCCTTGTAGAAGC
GCGT
UL27 promoter
[SEQ ID NO.: 416]
TCAACGGGCCCCTCTTTGATCACTCCACCCACAGCTTCGCCCAGCCCCC
CAACACCGCGCTGTATTACAGCGTCGAGAACGTGGGGCTCCTGCCGCAC
CTGAAGGAGGAGCTCGCCCGGTTCATCATGGGGGCGGGGGGCTCGGGTG
CTGATTGGGCCGTCAGCGAATTTCAGAGGTTTTACTGTTTTGACGGCAT
TTCCGGAATAACGCCCACTCAGCGCGCCGCCTGGCGATATATTCGCGAG
CTGATTATCGCCACCACACTCTTTGCCTCGGTCTACCGGTGCGGGGAGC
TCGAGTTGCGCCGCCCGGACTGCAGCCGCCCGACCTCCGAAGGTCGTTA
CCGTTACCCGCCCGGCGTATATCTCACGTACGACTCCGACTGTCCGCTG
GTGGCCATCGTCGAGAGCGCCCCCGACGGCTGTATCGGCCCCCGGTCGG
TCGTGGTCTACGACCGAGACGTTTTCTCGATCCTCTACTCGGTCCTCCA
GCACCTCGCCCCCAGGCTACCTGACGGGGGGCACGACGGGCCCCCGTAG
TCCCGCC
UL29 promoter
[SEQ ID NO.: 417]
CGGGCGGCGAGCTGCTGCGCGGCGCCCCGGCCGGCGGCCCGGTTTATTC
GCGTCGGCCCGGCCGGCCGGGCTTATGGACCGCCGGCGGCCGACAGGAG
AGTGACGTAGCCGGTGGGCGTGGAGGGGCTGGGGCGGACCGGCACGCCC
CCAGGTAAAGTGTACATATACCAACCGCATACCAGACGCACCCGACCCG
GAGCACCTGACCGTAAGCATCTGTGCCTCTCGCAGGGACCCCGCGTTGC
CGGCCGCCGGGGTTCATCGGCACCCCGTGGTTACCCGGGGGGTTGTCGG
TGAAGGGGAGGGATTCATTCCCCAACCCCGGTCTCCAACCCTCCCCTTG
ACCGTCGCCGCCCCCCCCCGGATTTTGACGCTCGGGAGACATACCTTGT
CGGGCGTCCGTCGTCGTGCCGGGATTACCTCCGTTCGCGGACCGATTGA
CAAAAGGAC
UL37 promoter
[SEQ ID NO.: 418]
CCCGGGCCTGGGTCCGCGAACGGGATGCCGGGACTTAAGTGGCCGTATA
ACACCCCGCGAAGACGCGGGGTACTCGCAACGCCTGCGGGGGTCCTGGA
GGGCCGCGGGGGATCGATAATTCGCCGCTCCCTACAGCGCACGACAGTC
ATTCCCGCCCGGTCTCGTCGTTGGTCTACGCTGTCCCCCACCCACGCGA
GCCGGGCGTC
UL38 promoter
[SEQ ID NO.: 419]
CCCGGGGGATTGTCCGGATGTGCGGGCAGCCCGGACGGCGTGGGTTGCG
GACTTTCTGCGGGGCGGCCCAAATGGCCCTTTAAACGTGTGTATACGGA
CGCGCCGGGCCAGTCGGCCAACACAACCCACCGGAGGCGGTAGCCGCGT
TTGGCTGTGGGGTGGGTGGTTCCGCCTTGCGTGAGTGTCCTTTCGACCC
CCCCCCCCCTCCCTCCCCCGGGTCTTGCTAGGTCGCGATCTGGGGTCGC
A
UL39 promoter
[SEQ ID NO.: 420]
CCGCTGTCACTCGTTGTTCGTTGACCCGGGCGTCCGCCAAATAAAGCCA
CTGAAACCCGAAACGCGAGTGTTGTAACGTCCTTTGGGCGGGAGGAAGC
CACAAAATGCAAATGGGATACATGGAAGGAACACACCCCCGTGACTCAG
GACATCGGTGTGTCCTTTTGGGTTTCACTGAAACTGGCCCGCGCCCCAC
TCCCTGCGCGATGTGGATAAAAAGCCAGCGCGGGTGGTTAGGGTACCAC
AGGTGGGTGCTTTGGAAACTTGCCGGTCGCCGTGCTCCTGTGAGCTTGC
GTCCCTCCCCGGTTTCCTTTGCGCTCCCGCCTTCCGGACCTGCTCTCGC
CTATCTTCTTTGGCTCTCGGTGCGATTCGTCAGGCAGCGGCCTTGTCGA
ATCTCGACCCCACCACTCGCCGGACCCGCCGACGTCCCCTCTCGAGCCC
GCCGAAACCCGCCGCGTCTGTTGAA
UL42 promoter
[SEQ ID NO.: 421]
ATTTCGATGGCCCAACTCCACGCGGATTGGTGCAGCACCCTGCATGCGC
CGGTGCGGGCCAACCTTCCCCCCGCTCATTGCCTCTTCCAAAAGGGTGT
GGCCTAACGAGCTGGGGGCGTATTTAATCAGGCTAGCGCGGCGGGCCTG
CCGTAGTTTCTGGCTCGGTGAGCGACGGTCCGGTTGCTTGGGTCCCCTG
GCTGCCATCAAAACCCCACCCTCGCAGCGGCATACGCCCCCTCCGCGTC
CCGCACCCGAGACCCCGGCCCGGCTGCCCTCACCACCGAAGCCCACCTC
GTCACTGTGGGGTGTTCCCAGCCCGCGTTGGG
UL44 promoter
[SEQ ID NO.: 422]
GCAGGTCATCAACCTCGGGTTGGTGTTTCGGTTTTCCGAGGTTGTCGTG
TATGCGGCGCTAGGGGGTGCCGTGTGGATTTCGTTGGCGCAGGTGCTGG
GGCTCCGGCGTCGCCTGCACAGGAAGGACCCCGGGGACGGGGCCCGGTT
GGCGGCGACGCTTCGGGGCCTCTTCTTCTCCGTGTACGCGCTGGGGTTT
GGGGTGGGGGTGCTGCTGTGCCCTCCGGGGTCAACGGGCGGGCGGTCGG
GCGATTGATATATTTTTCAATAAAAGGCATTAGTCCCGAAGACCGCCGG
TGTGTGATGATTTCGCCATAACACCCAAACCCCGGATGGGGCCCGGGTA
TAAATTCCGGAAGGGGACACGGGCTACCCTCACTATCGAGGGCGCTTGG
TCGGGAGGCCGCATCGAACGCACACCCCCATCCGGTGGTCCGTGTGGAG
GTCGTTTTCAGTGCCCGGTCTCGCTTTGCCGGGAACGCTAGCCGATCCC
TCGCGAGGGGGAGGCGTCGGGC
UL48 promoter
[SEQ ID NO.: 423]
GGGGTTCATTCGGTGTTGGCGTTGCGTGCCTTTGTTTCCCAATCCGACG
GGGACCGGGACTGGGTGGCGGGGGGTGGGTTGGACAGCCGCCCTCGGTT
CGCCTTCACGTGACAGGAGCCAATGTGGGGGGAAGTCACGAGGTACGGG
GCGGCCCGTGCGGGTTGCTTAAATGCGTGGTGGCGACCACGGGCTGTCA
TTCCTCGGGAACGGACGGGGTTCCCGCTGCCCACTTCCCCCCATAAGGT
CCGTCCGGTCCTCTAACGCGTTTGGGGGTTTTCTCTTCCCGCGCCGTCG
GGCGTCCCACACTCTCTGGGCGGGCGGGGACGATCGCATCAAAAGCCCG
ATATCGTCTTTCCCGTATCAACCCCACCCA
UL54 promoter
[SEQ ID NO.: 424]
ACCCCGCCCATGGGTCCCAATTGGCCGTCCCGTTACCAAGACCAACCCA
GCCAGCGTATCCACCCCCGCCCGGGTCCCCGCGGAAGCGGAACGGGGTA
TGTGATATGCTAATTAAATACATGCCACGTACTTATGGTGTCTGATTGG
TCCTTGTCTGTGCCGGAGGTGGGGCGGGGGCCCCGCCCGGGGGGCGGAA
CGAGGAGGGGTTTGGGAGAGCCGGCCCCGGCACCACGGGTATAAGGACA
TCCACCACCCGGCCGGTGGTGGTGTGCAGCCGTGTTCCAACCACGGTCA
CGCTTCGGTGCCTCTCCCCGATTCGGGCCCGGTCGCTCGCTACCGGTGC
GCCACCACCAGAGGCCATATCCGACACCCCAGCCCCGACGGCAGCCGAC
AGCCCGGTC
US6 promoter
[SEQ ID NO.: 425]
CTGCTTGAGCTCCTGCGTCGTACGTGCCGCGGGTGGGGGCGTTACCATC
CCTACATGGACCCAGTTGTCGTATAATTTCCCCCCCCCCCCCCCTTCTC
CGCGTGGGTGATGTCGGGTCCAAACTCCCGACACCACCAGCTGGCATGG
TATAAATCACCGGTGCGCCCCCCAAACCATGTCCGGCAGGGGGATGGGG
GGGCGAATGCGGAGGGCACCCAACAACACCGGGCTAACCAGGAAATCCG
TGGCCCCGGCCCCCAATAAAGATCGCGGTAGCCCGGCCGTGTGACACTA
TCGTCCATACCGACCACACCGACGAATCCCCCAAGGGGGAGGGGCCATT
TTACGAGGAGGAGGGGTATAACAAAGTCTGTCTTTAAAAAGCAGGGGTTA
GGGAGTTGTTCGGTCATAAGCTTCAGCGCGAACGACCAACTACCCCGATC
ATCAGTTATCCTTAAGGTCTCTTTTGTGTGGTGCGTTCCGGT

The present disclosure further provides compositions comprising such gene editing systems, vector encoding such gene editing systems, and use of such gene editing systems, compositions and vectors, e.g., where the expression of at least one component of the genome editing system disclosed herein (e.g., at least one gRNA molecule, and/or at least one RNA-guided nuclease) is regulated by a promoter derived from a genome of HSV-1, and/or HSV-2.

In certain embodiments, the genome editing system is a ZFN system. The ZFN can act as restriction enzyme, which is generated by combining a zinc finger DNA-binding domain with a DNA-cleavage domain. A zinc finger domain can be engineered to target specific DNA sequences, which allows the zinc-finger nuclease to target desired sequences within genomes. The DNA-binding domains of individual ZFNs typically contain a plurality of individual zinc finger repeats and can each recognize a plurality of base pairs. The most common method to generate new zinc-finger domain is to combine smaller zinc-finger “modules” of known specificity. The most common cleavage domain in ZFNs is the non-specific cleavage domain from the type IIs restriction endonuclease FokI. ZFN modulates the expression of proteins by producing double-strand breaks (DSBs) in the target DNA sequence, which will, in the absence of a homologous template, be repaired by non-homologous end-joining (NHEJ). Such repair may result in deletion or insertion of base-pairs, producing frame-shift and preventing the production of the harmful protein (Durai et al., Nucleic Acids Res.; 33 (18): 5978-90.) Multiple pairs of ZFNs can also be used to completely remove entire large segments of genomic sequence (Lee at al., Genome Res.; 20 (1): 81-9).

In certain embodiments, the genetic engineering system is a transcription activator-like effector nuclease (TALEN) system. TALENs are restriction enzymes that can be engineered to cut specific sequences of DNA. TALEN systems operate on a similar principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome (Boch et al., Nature Biotechnology; 29(2):135-6).

In certain embodiments, the genetic engineering system is a meganuclease system. Meganucleases are endodeoxyribonucleases that can recognize large sites of DNA sequences, for example, 12-40 base-pair long. Because these large recognition sites generally only occur once in a genome, meganucleases can be used for genome engineering and gene editing, by targeting a particular gene or a particular sequence of the genome, and editing the genome or the gene (Redondo et al., Nature; 456:107-111). In certain embodiments, the meganuclease is a natural occurring meganuclease. In certain embodiment, the meganuclease is a genetically-engineered meganuclease that is created through rational design and selection, and is engineered to target novel sequences Non-limiting examples of meganucleases include I-TevI, I-SceI, and PI-SceI.

In certain embodiments, the genetic engineering system is a MegaTAL system. MegaTAL refers to a group of hybrid endonuclease that are derived from the fusion of a meganuclease with a TAL effector. Advantages of MegaTALs include having high rates of DNA modification with high target site specificity. In certain embodiments, the MegaTAL comprises a TAL effector domain and a cleavage sequence of a meganuclease cleavage domain (Boissel et al., Nucleic Acid Research; 42(4):2591-2601).

Based on the instant disclosure, additional suitable nucleases or nuclease systems will be apparent to those of skill in the art.

Guide RNA (gRNA) Molecules

The terms “guide RNA” and “gRNA” refer to any nucleic acid that promotes the specific association (or “targeting”) of an RNA-guided nuclease such as a Cas9 or a Cpf1 to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (Briner), which is incorporated by reference), and in Cotta-Ramusino.

In bacteria and archaea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. This duplex can facilitate the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). (Mali et al. Science. 2013 Feb. 15; 339(6121): 823-826 (“Mali”); Jiang et al. Nat Biotechnol. 2013 March; 31(3): 233-239 (“Jiang”); and Jinek et al., 2012 Science August 17; 337(6096): 816-821 (“Jinek”), all of which are incorporated by reference herein.)

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu et al., Nat Biotechnol. 2013 September; 31(9): 827-832, (“Hsu”), incorporated by reference herein), “complementarity regions” (Cotta-Ramusino), “spacers” (Briner) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that can influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes. (Nishimasu et al., Cell 156, 935-949, Feb. 27, 2014 (Nishimasu 2014) and Nishimasu et al., Cell 162, 1113-1126, Aug. 27, 2015 (Nishimasu 2015), both incorporated by reference herein). It should be noted that the first and/or second complementarity domains can contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner.

While the foregoing description has focused on gRNAs for use with Cas9, it should be appreciated that other RNA-guided nucleases have been (or can in the future be) discovered or invented which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (Zetsche I), incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Those of skill in the art will appreciate that, although structural differences can exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs can be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

gRNA Design

Methods for selection and validation of target sequences as well as off-target analyses have been described previously, e.g., in Mali; Hsu; Fu et al., 2014 Nat biotechnol 32(3): 279-84, Heigwer et al., 2014 Nat methods 11(2):122-3; Bae et al. (2014) Bioinformatics 30(10): 1473-5; and Xiao A et al. (2014) Bioinformatics 30(8): 1180-1182. Each of these references is incorporated by reference herein. In certain non-limiting embodiment, gRNA design can involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

In certain embodiments, gRNAs targeting herpes viral genes can be further screened using primary in vitro cell-based gRNA screen. In certain embodiments, candidate gRNAs can be complexed into ribonucleoproteins (RNP), and then delivered to susceptible or permissive cells through a vector. In certain embodiments, the vector is a lipofection reagent. The susceptible or permissible cells are then infected with herpes virus, and the gRNAs' ability to inhibit viral replication can be evaluated by measuring the production of virions from the infected cells. Non-limiting exemplary vitro cell lines that can be used with the disclosed invention are MRCS, HFF, Vero, Vero 76, HEK293T and other epithelial cell lines. In certain embodiments, cells are seeded at a density ranging from about 5,000-20,000 cells per well of a 96-well plate. In certain embodiments, RNP transfection dose can range from about 10 uM to about 1 fM.

In certain embodiments, the virion production can be measured by qPCR as the secreted virion DNA copy number. Other exemplary assays measuring virion production include, but are not limited to, plaque assay, Western Blot, ELISA, Non-Homologous End Joining/Next-Generation Sequencing (NHEJ/NGS). Exemplary susceptible and permissible cells include, but not limited to, 293T, Vero, and Hela cells. Exemplary herpesviruses include, but not limited to, HSV-1, HSV-2., CMV, KSHV, VZV, and EBV.

In certain embodiments, the RNPs disclosed herein have minimal or no off-target effects. In certain embodiments, the off-target effect of an RNP is measured by Digenome-seq analysis (Kim et al., Nature Methods (2015); 12:237-243). In certain embodiments, the off-target effect of an RNA is indicated by an off-target count as measured by Digenome-seq analysis. In certain embodiments, the off-target count is measured by Digenome-seq analysis at 1000 nM of the RNP. In certain embodiments, the off-target count is measured by the Digenome-seq analysis at 100 nM of the RNP.

In certain embodiments, the off-target count as measured Digenome-seq analysis of the RNPs disclosed herein at 1000 nM is less than about 20, less than about 19, less than about 18, less than about 17, less than about 16, less than about 15, less than about 14, less than about 13, less than about 12, less than about 11, less than about 10, less than about 9, less than about 8, less than about 7, less than about 6, less than about 5, less than about 4, less than about 3, less than about 2, or less than about 1. In certain embodiments, the off-target count of the RNPs disclosed herein as measured Digenome-seq at 1000 nM is zero or is about zero. In certain embodiments, the off-target count of the RNPs disclosed herein as measured Digenome-seq at 100 nM is less than about 20, less than about 19, less than about 18, less than about 17, less than about 16, less than about 15, less than about 14, less than about 13, less than about 12, less than about 11, less than about 10, less than about 9, less than about 8, less than about 7, less than about 6, less than about 5, less than about 4, less than about 3, less than about 2, or less than about 1. In certain embodiments, the off-target count of the RNPs disclosed herein as measured Digenome-seq at 100 nM is zero or is about zero.

Exemplary Targeting Domains for Knockout of HSV-1 Genes.

Exemplary targeting domains of the gRNAs targeting one or more HSV-1 genes (e.g., essential HSV-1 viral gene(s)) are provided in SEQ ID NO.: 1-411. In certain embodiments, the targeting domains of the gRNAs targeting one or more HSV-1 genes are selected from SEQ ID NO.: 1-54, 410, and 411. In certain embodiments, the targeting domains of the gRNAs targeting one or more HSV-1 genes are selected from SEQ ID NO.: 1-25, 410, and 411. In certain embodiments, the targeting domains of the gRNAs targeting one or more HSV-1 genes are selected from SEQ ID NO.: 1-14, 410, and 411. In certain embodiments, the targeting domains of the gRNAs targeting one or more HSV-1 genes are selected from Table 2.

In certain embodiments, the targeting domains start with a 5′G, e.g., in the C-terminal domain. In certain embodiments, the targeting domain hybridizes to the target domain on a HSV-1 gene through complementary base pairing. Any of the targeting domains disclosed in Table 2 can be used with a S. aureus Cas9 molecule that generates a double-stranded break (Cas9 nuclease) or a single-stranded break (Cas9 nickase).

In certain embodiments, two or more (e.g., three, four, or five) gRNA molecules are used with one Cas9 molecule. In certain embodiments, two or more (e.g., three, four, or five) gRNAs are used with two or more Cas9 molecules, and at least one Cas9 molecule can be from a different species than the other Cas9 molecule(s). For example, when two gRNA molecules are used with two Cas9 molecules, one Cas9 molecule can be from one species and the other Cas9 molecule can be from a different species. Both Cas9 species can be used to generate a single or double-strand break, as desired.

Any of the targeting domains provided in SEQ ID NO.: 1-411 can be used with a Cas9 nickase molecule to generate a single strand break.

Any of the targeting domains provided in SEQ ID NO.: 1-411 can be used with a Cas9 nuclease molecule to generate a double strand break.

In certain embodiments, any upstream gRNA described herein can be paired with any downstream gRNA described herein. When an upstream gRNA designed for use with one species of Cas9 is paired with a downstream gRNA designed for use from a different species of Cas9, both Cas9 species are used to generate a single or double-strand break, as desired.

TABLE 2

| SEQ | gRNA | Target | |
|---|---|---|---|
| ID No. | ID | Gene | Targeting domain |
| 1 | 177 | RL2 | GGAGGCCGCCGAGGACGUCAG |
| 2 | 098 | RS1 | GUCAUCGACCUCGUCGGACU |
| 3 | 626 | UL6 | GUCUGUCAGGUACGUUUGCAG |
| 4 | 390 | UL15 | GCUGCUCGAGCAGAUACUCGA |
| 5 | 411 | UL19 | GUCGGGCUGCAUAAACUGCUC |
| 6 | 441 | UL22 | GACUUCGACCGGGGUCUCGGA |
| 7 | 480 | UL29 | GCGUGAUGGACAUGUUUAACA |
| 8 | 504 | UL32 | GCCCGUCUCCUGGACUGCACG |
| 9 | 514 | UL33 | GCGCUGCGGUCCCAGACCCUG |
| 10 | 524 | UL37 | GCCAGAACGUAGGGGACGAAGC |
| 11 | 570 | UL48 | GGCGAGGUCGUGAAGCUGGAAU |
| 12 | 596 | UL54 | GCCCUUUGACGCCGAGACCAG |
| 13 | 097 | RL2 | GAGGCCGCCGAGGACGUCAG |
| 14 | 571 | UL48 | GCGAGGUCGUGAAGCUGGAAU |
| 14 | 099 | UL48 | GCGAGGUCGUGAAGCUGGAAU |
| 15 | 272 | RL2 | GCUCAGGCCGCGAACCAAGAA |
| 16 | 345 | RS1 | GGCGGCGGCCCGUCGGUGGGGCC |
| 17 | 385 | UL15 | GUGCGUGGCGAUGGCGACGG |
| 18 | 407 | UL19 | GCCUGCAGCAGGUAAAACACCG |
| 19 | 466 | UL22 | GGUGCGUGAUGUCCAGCUCCGUCG |
| 20 | 479 | UL29 | GCGCGUGAUGGACAUGUUUAACA |
| 21 | 503 | UL32 | GCGCCCGUCUCCUGGACUGCACG |
| 22 | 535 | UL37 | GCACUACCUUUCGGCCCUGG |
| 23 | 551 | UL37 | GUUCUUGGCCCUGGUGAGUAA |
| 24 | 100 | UL54 | GACCUGGAAUCGGACAGCAGCG |
| 25 | 616 | UL6 | GGGAGGACGACGAGACCAAGG |
| 26 | 178 | RL2 | GCGGAGGCCGCCGAGGACGUCAG |
| 27 | 625 | UL6 | GAUGUCUGUCAGGUACGUUUGCAG |
| 28 | 410 | UL19 | GUUGUCGGGCUGCAUAAACUGCUC |
| 29 | 271 | RL2 | GGCUCAGGCCGCGAACCAAGAA |
| 30 | 398 | UL19 | GGGGUGCGCGGGGUCCGUCA |
| 31 | 406 | UL19 | GGCCUGCAGCAGGUAAAACACCG |
| 32 | 285 | RL2 | GCCCAUCCUGGACAUGGAGAC |
| 33 | 573 | UL48 | GUCAACGGAGCGCUCACCGUCCG |
| 34 | 614 | UL6 | GACGUGCGCGUGUUCCCGGA |
| 35 | 509 | UL32 | GACUGCGCCAGGAACGAGUAGU |
| 36 | 613 | UL6 | GCAGACGUGCGCGUGUUCCCGGA |
| 37 | 564 | UL37 | GCUCUUGGCCGAGAACCUCC |
| 38 | 536 | UL37 | GCACGCACUACCUUUCGGCCCUGG |
| 39 | 508 | UL32 | GCCAAGGUUCCGAUGAGGGC |
| 40 | 617 | UL6 | GAUUCGGCAAGGGCGGAAACAGCC |
| 41 | 305 | RL2 | GCAGGUAGCGCGUGAGGCCGCCCG |
| 42 | 416 | UL19 | GUACGGAUUGGCGGCUUCGA |
| 43 | 440 | UL22 | GGACUUCGACCGGGGUCUCGGA |
| 44 | 426 | UL19 | GUAAUUGUGUGUGGGUUGCUCGA |
| 45 | 569 | UL48 | GGGCGAGGUCGUGAAGCUGGAAU |
| 46 | 584 | UL48 | GUACCAUGCUCGAUACCUGGAAC |
| 47 | 494 | UL29 | GUCCACCUCGGUGAGAUGGAGGG |
| 48 | 615 | UL6 | GGCAGACGUGCGCGUGUUCCCGGA |
| 49 | 417 | UL19 | GCCGCGUCAACGUUCAUCAGCG |
| 50 | 430 | UL19 | GCUCCAGAAAGCGCACGAGCGA |
| 51 | 337 | RS1 | GUUGCGCGCGGCGCCCGAGAU |
| 52 | 449 | UL22 | GGGCGGCCAACCCGGCAAGCGCGC |
| 53 | 391 | UL19 | GCGCUGACCUACGCGCUCAUGG |
| 54 | 572 | UL48 | GCUGGAAUACGAGUCCAACUUCGC |
| 228 | 619 | UL6 | GUCCUGGGCGUACGAAGGGAUGU |
| 157 | 620 | UL6 | GGUCCUGGGCGUACGAAGGGAUGU |
| 175 | 621 | UL6 | GCUCCAGGUCCUGGGCGUACGA |
| 198 | 622 | UL6 | GCGCUCCAGGUCCUGGGCGUACGA |
| 102 | 623 | UL6 | GAAUGCGGCGAUCGCCCCGC |
| 92 | 624 | UL6 | GACGAAUGCGGCGAUCGCCCCGC |
| 65 | 388 | UL15 | GUGCGCCUGCAGACCGACCCGG |
| 68 | 389 | UL15 | GCGCCUGCAGACCGACCCGG |
| 88 | 408 | UL19 | GCCGUUCACCACGCGCCAGGUGGC |
| 141 | 409 | UL19 | GUUCACCACGCGCCAGGUGGC |
| 169 | 438 | UL22 | GGCCAGGACUUCGACCGGGGUC |
| 186 | 439 | UL22 | GCCAGGACUUCGACCGGGGUC |
| 96 | 523 | UL37 | GGCCAGAACGUAGGGGACGAAGC |
| 57 | 525 | UL37 | GAAGCCCGGGUCGGCGAGGACGU |
| 62 | 527 | UL37 | GCCCGGGUCGGCGAGGACGU |
| 83 | 574 | UL48 | GACCUGGAGAGCUGGCGUCAGUUG |
| 301 | 595 | UL54 | GGCCCUUUGACGCCGAGACCAG |
| 369 | 481 | UL6 | GACGAAUGCGGCGAUCGCCCCGC |
| 410 | 208 | RL2 | GAGGCCGCCGAGGACGUCAGGGGGGU |
| 411 | 135 | UL48 | GCGAGGUCGUGAAGCUGGAAUACGAGU |

gRNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, can be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5)ppp(5)G cap analog, a m7G(5)ppp(5)G cap analog, or a 3′-O-Me-m7G(5)ppp(5)G anti reverse cap analog (ARCA)), as shown below:

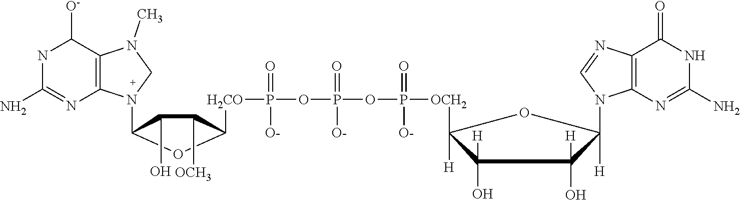

The cap or cap analog can be included during either chemical synthesis or in vitro transcription of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g. a gRNA, whether transcribed in vivo from a DNA vector, or in vitro transcribed gRNA, can include either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

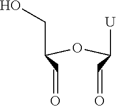

wherein “U” can be an unmodified or modified uridine.

The 3′ terminal U ribose can be modified with a 2′3′ cyclic phosphate as shown below:

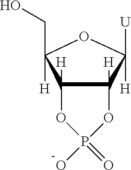

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA are deoxynucleotides.

Non-limiting exemplary strategies, methods, and compositions suitable for editing a target nucleic acid sequence, or modulating expression of a target nucleic acid sequence, e.g., a target nucleic acid sequence of a herpes simplex virus type 1 (HSV-1) gene or genome, have been disclosed herein. Based on the instant disclosure, additional suitable strategies, methods, and compositions will be apparent to those of skill in the art. It will be understood, for example, and without limitation, that guide RNAs other than those exemplified herein can be used for editing a target nucleic acid sequence, or modulating expression of a target nucleic acid sequence, e.g., a target nucleic acid sequence of a herpes simplex virus type 1 (HSV-1) gene or genome. Non-limiting exemplary methods for designing guide RNAs are disclosed herein and additional suitable methods will be apparent to the skilled artisan based on the present disclosure and the knowledge in the art. In certain embodiments envisioned, a guide RNA is designed and/or used in a strategy, method, and/or compositions for editing or modulating expression of a target nucleic acid sequence of a HSV-1 gene or genome that binds to a target site within about 5 nucleotides, within about 10 nucleotides, within about 20 nucleotides, within about 25 nucleotides, within about 30 nucleotides, within about 40 nucleotides, within about 50 nucleotides, within about 60 nucleotides, within about 70 nucleotides, within about 75 nucleotides, within about 80 nucleotides, within about 90 nucleotides, within about 100 nucleotides, within about 200 nucleotides, within about 250 nucleotides, within about 300 nucleotides, within about 400 nucleotides, within about 500 nucleotides, within about 600 nucleotides, within about 700 nucleotides, within about 750 nucleotides, within about 800 nucleotides, within about 900 nucleotides, or within about 1000 nucleotides from the target site of any of the guide RNAs disclosed herein.

For example, in certain embodiments, a guide RNA is designed and/or used in a strategy, method, and/or compositions for editing or modulating expression of a target nucleic acid sequence of a HSV-1 gene or genome that binds to a target site within about 5 nucleotides, within about 10 nucleotides, within about 20 nucleotides, within about 25 nucleotides, within about 30 nucleotides, within about 40 nucleotides, within about 50 nucleotides, within about 60 nucleotides, within about 70 nucleotides, within about 75 nucleotides, within about 80 nucleotides, within about 90 nucleotides, within about 100 nucleotides, within about 200 nucleotides, within about 250 nucleotides, within about 300 nucleotides, within about 400 nucleotides, within about 500 nucleotides, within about 600 nucleotides, within about 700 nucleotides, within about 750 nucleotides, within about 800 nucleotides, within about 900 nucleotides, or within about 1000 nucleotides from the target site of any of the guide RNAs having sequences set forth in SEQ ID NOs: 1-411.

In certain embodiments, a guide RNA is designed and/or used in a strategy, method, and/or compositions for editing or modulating expression of a target nucleic acid sequence of a HSV-1 gene or genome that binds to a target site within about 5 nucleotides, within about 10 nucleotides, within about 20 nucleotides, within about 25 nucleotides, within about 30 nucleotides, within about 40 nucleotides, within about 50 nucleotides, within about 60 nucleotides, within about 70 nucleotides, within about 75 nucleotides, within about 80 nucleotides, within about 90 nucleotides, within about 100 nucleotides, within about 200 nucleotides, within about 250 nucleotides, within about 300 nucleotides, within about 400 nucleotides, within about 500 nucleotides, within about 600 nucleotides, within about 700 nucleotides, within about 750 nucleotides, within about 800 nucleotides, within about 900 nucleotides, or within about 1000 nucleotides from the target site of any of the guide RNAs having sequences set forth in SEQ ID NOs: 1-54, 410, and 411.

In certain embodiments, a guide RNA is designed and/or used in a strategy, method, and/or compositions for editing or modulating expression of a target nucleic acid sequence of a HSV-1 gene or genome that binds to a target site within about 5 nucleotides, within about 10 nucleotides, within about 20 nucleotides, within about 25 nucleotides, within about 30 nucleotides, within about 40 nucleotides, within about 50 nucleotides, within about 60 nucleotides, within about 70 nucleotides, within about 75 nucleotides, within about 80 nucleotides, within about 90 nucleotides, within about 100 nucleotides, within about 200 nucleotides, within about 250 nucleotides, within about 300 nucleotides, within about 400 nucleotides, within about 500 nucleotides, within about 600 nucleotides, within about 700 nucleotides, within about 750 nucleotides, within about 800 nucleotides, within about 900 nucleotides, or within about 1000 nucleotides from the target site of any of the guide RNAs having sequences set forth in SEQ ID NOs: 1-25, 410, and 411.

In certain embodiments, a guide RNA is designed and/or used in a strategy, method, and/or compositions for editing or modulating expression of a target nucleic acid sequence of a HSV-1 gene or genome that binds to a target site within about 5 nucleotides, within about 10 nucleotides, within about 20 nucleotides, within about 25 nucleotides, within about 30 nucleotides, within about 40 nucleotides, within about 50 nucleotides, within about 60 nucleotides, within about 70 nucleotides, within about 75 nucleotides, within about 80 nucleotides, within about 90 nucleotides, within about 100 nucleotides, within about 200 nucleotides, within about 250 nucleotides, within about 300 nucleotides, within about 400 nucleotides, within about 500 nucleotides, within about 600 nucleotides, within about 700 nucleotides, within about 750 nucleotides, within about 800 nucleotides, within about 900 nucleotides, or within about 1000 nucleotides from the target site of any of the guide RNAs having sequences set forth in SEQ ID NOs: 1-14, 410, and 411.

In certain embodiments, a guide RNA is designed and/or used in a strategy, method, and/or compositions for editing or modulating expression of a target nucleic acid sequence of a HSV-1 gene or genome that binds to a target site within about 5 nucleotides, within about 10 nucleotides, within about 20 nucleotides, within about 25 nucleotides, within about 30 nucleotides, within about 40 nucleotides, within about 50 nucleotides, within about 60 nucleotides, within about 70 nucleotides, within about 75 nucleotides, within about 80 nucleotides, within about 90 nucleotides, within about 100 nucleotides, within about 200 nucleotides, within about 250 nucleotides, within about 300 nucleotides, within about 400 nucleotides, within about 500 nucleotides, within about 600 nucleotides, within about 700 nucleotides, within about 750 nucleotides, within about 800 nucleotides, within about 900 nucleotides, or within about 1000 nucleotides from the target site of any of the guide RNAs disclosed herein to show efficacy in editing or modulating, e.g., reducing or abolishing, expression of a target nucleic acid sequence of a HSV-1 gene or genome.

In certain embodiments, the guide RNA is designed and/or used in a strategy, method, and/or compositions for editing or modulating expression of a target nucleic acid sequence of a HSV-1 gene or genome that binds to a target site between the target site of gRNA 097 and the target site of gRNA 178. In certain embodiments, the guide RNA is designed and/or used in a strategy, method, and/or compositions for editing or modulating expression of a target nucleic acid sequence of a HSV-1 gene or genome that binds to a target site between the target site of gRNA 097 and the target site of gRNA 177. In certain embodiments, the guide RNA is designed and/or used in a strategy, method, and/or compositions for editing or modulating expression of a target nucleic acid sequence of a HSV-1 gene or genome that binds to a target site between the target site of gRNA 097 and the target site of gRNA 177.

In certain embodiments, the guide RNA is designed and/or used in a strategy, method, and/or compositions for editing or modulating expression of a target nucleic acid sequence of a HSV-1 gene or genome that binds to a target site between the target site of gRNA 619 and the target site of gRNA 625. In certain embodiments, the guide RNA is designed and/or used in a strategy, method, and/or compositions for editing or modulating expression of a target nucleic acid sequence of a HSV-1 gene or genome that binds to a target site between the target site of gRNA 620 and the target site of gRNA 625. In certain embodiments, the guide RNA is designed and/or used in a strategy, method, and/or compositions for editing or modulating expression of a target nucleic acid sequence of a HSV-1 gene or genome that binds to a target site between the target site of gRNA 621 and the target site of gRNA 625. In certain embodiments, the guide RNA is designed and/or used in a strategy, method, and/or compositions for editing or modulating expression of a target nucleic acid sequence of a HSV-1 gene or genome that binds to a target site between the target site of gRNA 621 and the target site of gRNA 625. In certain embodiments, the guide RNA is designed and/or used in a strategy, method, and/or compositions for editing or modulating expression of a target nucleic acid sequence of a HSV-1 gene or genome that binds to a target site between the target site of gRNA 622 and the target site of gRNA 625. In certain embodiments, the guide RNA is designed and/or used in a strategy, method, and/or compositions for editing or modulating expression of a target nucleic acid sequence of a HSV-1 gene or genome that binds to a target site between the target site of gRNA 623 and the target site of gRNA 625. In certain embodiments, the guide RNA is designed and/or used in a strategy, method, and/or compositions for editing or modulating expression of a target nucleic acid sequence of a HSV-1 gene or genome that binds to a target site between the target site of gRNA 624 and the target site of gRNA 625. In certain embodiments, the guide RNA is designed and/or used in a strategy, method, and/or compositions for editing or modulating expression of a target nucleic acid sequence of a HSV-1 gene or genome that binds to a target site between the target site of gRNA 626 and the target site of gRNA 625. In certain embodiments, the guide RNA is designed and/or used in a strategy, method, and/or compositions for editing or modulating expression of a target nucleic acid sequence of a HSV-1 gene or genome that binds to a target site between the target site of gRNA 619 and the target site of gRNA 626. In certain embodiments, the guide RNA is designed and/or used in a strategy, method, and/or compositions for editing or modulating expression of a target nucleic acid sequence of a HSV-1 gene or genome that binds to a target site between the target site of gRNA 620 and the target site of gRNA 626. In certain embodiments, the guide RNA is designed and/or used in a strategy, method, and/or compositions for editing or modulating expression of a target nucleic acid sequence of a HSV-1 gene or genome that binds to a target site between the target site of gRNA 621 and the target site of gRNA 626. In certain embodiments, the guide RNA is designed and/or used in a strategy, method, and/or compositions for editing or modulating expression of a target nucleic acid sequence of a HSV-1 gene or genome that binds to a target site between the target site of gRNA 621 and the target site of gRNA 626. In certain embodiments, the guide RNA is designed and/or used in a strategy, method, and/or compositions for editing or modulating expression of a target nucleic acid sequence of a HSV-1 gene or genome that binds to a target site between the target site of gRNA 622 and the target site of gRNA 626. In certain embodiments, the guide RNA is designed and/or used in a strategy, method, and/or compositions for editing or modulating expression of a target nucleic acid sequence of a HSV-1 gene or genome that binds to a target site between the target site of gRNA 623 and the target site of gRNA 626. In certain embodiments, the guide RNA is designed and/or used in a strategy, method, and/or compositions for editing or modulating expression of a target nucleic acid sequence of a HSV-1 gene or genome that binds to a target site between the target site of gRNA 624 and the target site of gRNA 626.

In certain embodiments, the guide RNA is designed and/or used in a strategy, method, and/or compositions for editing or modulating expression of a target nucleic acid sequence of a HSV-1 gene or genome that binds to a target site between the target site of gRNA 388 and the target site of gRNA 390. In certain embodiments, the guide RNA is designed and/or used in a strategy, method, and/or compositions for editing or modulating expression of a target nucleic acid sequence of a HSV-1 gene or genome that binds to a target site between the target site of gRNA 389 and the target site of gRNA 390.

In certain embodiments, the guide RNA is designed and/or used in a strategy, method, and/or compositions for editing or modulating expression of a target nucleic acid sequence of a HSV-1 gene or genome that binds to a target site between the target site of gRNA 438 and the target site of gRNA 411. In certain embodiments, the guide RNA is designed and/or used in a strategy, method, and/or compositions for editing or modulating expression of a target nucleic acid sequence of a HSV-1 gene or genome that binds to a target site between the target site of gRNA 439 and the target site of gRNA 411. In certain embodiments, the guide RNA is designed and/or used in a strategy, method, and/or compositions for editing or modulating expression of a target nucleic acid sequence of a HSV-1 gene or genome that binds to a target site between the target site of gRNA 440 and the target site of gRNA 411.

In certain embodiments, the guide RNA is designed and/or used in a strategy, method, and/or compositions for editing or modulating expression of a target nucleic acid sequence of a HSV-1 gene or genome that binds to a target site between the target site of gRNA 480 and the target site of gRNA 479. In certain embodiments, the guide RNA is designed and/or used in a strategy, method, and/or compositions for editing or modulating expression of a target nucleic acid sequence of a HSV-1 gene or genome that binds to a target site between the target site of gRNA 480 and the target site of gRNA 481

In certain embodiments, the guide RNA is designed and/or used in a strategy, method, and/or compositions for editing or modulating expression of a target nucleic acid sequence of a HSV-1 gene or genome that binds to a target site between the target site of gRNA 504 and the target site of gRNA 503.

In certain embodiments, the guide RNA is designed and/or used in a strategy, method, and/or compositions for editing or modulating expression of a target nucleic acid sequence of a HSV-1 gene or genome that binds to a target site between the target site of gRNA 523 and the target site of gRNA 527. In certain embodiments, the guide RNA is designed and/or used in a strategy, method, and/or compositions for editing or modulating expression of a target nucleic acid sequence of a HSV-1 gene or genome that binds to a target site between the target site of gRNA 523 and the target site of gRNA 525. In certain embodiments, the guide RNA is designed and/or used in a strategy, method, and/or compositions for editing or modulating expression of a target nucleic acid sequence of a HSV-1 gene or genome that binds to a target site between the target site of gRNA 523 and the target site of gRNA 524. In certain embodiments, the guide RNA is designed and/or used in a strategy, method, and/or compositions for editing or modulating expression of a target nucleic acid sequence of a HSV-1 gene or genome that binds to a target site between the target site of gRNA 524 and the target site of gRNA 525. In certain embodiments, the guide RNA is designed and/or used in a strategy, method, and/or compositions for editing or modulating expression of a target nucleic acid sequence of a HSV-1 gene or genome that binds to a target site between the target site of gRNA 524 and the target site of gRNA 527.

In certain embodiments, the guide RNA is designed and/or used in a strategy, method, and/or compositions for editing or modulating expression of a target nucleic acid sequence of a HSV-1 gene or genome that binds to a target site between the target site of gRNA 569 and the target site of gRNA 574. In certain embodiments, the guide RNA is designed and/or used in a strategy, method, and/or compositions for editing or modulating expression of a target nucleic acid sequence of a HSV-1 gene or genome that binds to a target site between the target site of gRNA 569 and the target site of gRNA 573. In certain embodiments, the guide RNA is designed and/or used in a strategy, method, and/or compositions for editing or modulating expression of a target nucleic acid sequence of a HSV-1 gene or genome that binds to a target site between the target site of gRNA 569 and the target site of gRNA 572. In certain embodiments, the guide RNA is designed and/or used in a strategy, method, and/or compositions for editing or modulating expression of a target nucleic acid sequence of a HSV-1 gene or genome that binds to a target site between the target site of gRNA 569 and the target site of gRNA 571. In certain embodiments, the guide RNA is designed and/or used in a strategy, method, and/or compositions for editing or modulating expression of a target nucleic acid sequence of a HSV-1 gene or genome that binds to a target site between the target site of gRNA 569 and the target site of gRNA 099. In certain embodiments, the guide RNA is designed and/or used in a strategy, method, and/or compositions for editing or modulating expression of a target nucleic acid sequence of a HSV-1 gene or genome that binds to a target site between the target site of gRNA 569 and the target site of gRNA 570. In certain embodiments, the guide RNA is designed and/or used in a strategy, method, and/or compositions for editing or modulating expression of a target nucleic acid sequence of a HSV-1 gene or genome that binds to a target site between the target site of gRNA 570 and the target site of gRNA 574. In certain embodiments, the guide RNA is designed and/or used in a strategy, method, and/or compositions for editing or modulating expression of a target nucleic acid sequence of a HSV-1 gene or genome that binds to a target site between the target site of gRNA 570 and the target site of gRNA 573. In certain embodiments, the guide RNA is designed and/or used in a strategy, method, and/or compositions for editing or modulating expression of a target nucleic acid sequence of a HSV-1 gene or genome that binds to a target site between the target site of gRNA 570 and the target site of gRNA 572. In certain embodiments, the guide RNA is designed and/or used in a strategy, method, and/or compositions for editing or modulating expression of a target nucleic acid sequence of a HSV-1 gene or genome that binds to a target site between the target site of gRNA 570 and the target site of gRNA 571. In certain embodiments, the guide RNA is designed and/or used in a strategy, method, and/or compositions for editing or modulating expression of a target nucleic acid sequence of a HSV-1 gene or genome that binds to a target site between the target site of gRNA 570 and the target site of gRNA 099.

In certain embodiments, the guide RNA is designed and/or used in a strategy, method, and/or compositions for editing or modulating expression of a target nucleic acid sequence of a HSV-1 gene or genome that binds to a target site between the target site of gRNA 595 and the target site of gRNA 596.

It will be understood that a target site within a certain number of nucleotides from a target site provided herein can be on the same DNA strand as the target site provided herein or on the DNA strand complementary thereto.

### RNA-Guided Nucleases

RNA-guided nucleases according to the present disclosure include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g. complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations can exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cpf1), species (e.g. S. pyogenes vs. S. aureus) or variation (e.g. full-length vs. truncated or split; naturally-occurring PAM specificity vs. engineered PAM specificity, etc.) of RNA-guided nuclease.

The PAM sequence takes its name from its sequential relationship to the “protospacer” sequence that is complementary to gRNA targeting domains (or “spacers”). Together with protospacer sequences, PAM sequences define target regions or sequences for specific RNA-guided nuclease/gRNA combinations.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov et al., 2015, Molecular Cell 60, 385-397, Nov. 5, 2015. It should also be noted that engineered RNA-guided nucleases can have PAM specificities that differ from the PAM specificities of reference molecules (for instance, in the case of an engineered RNA-guided nuclease, the reference molecule can be the naturally occurring variant from which the RNA-guided nuclease is derived, or the naturally occurring variant having the greatest amino acid sequence homology to the engineered RNA-guided nuclease).

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above) Ran & Hsu, et al., Cell 154(6), 1380-1389, Sep. 12, 2013 (Ran), incorporated by reference herein), or that that do not cut at all.

Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g. a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, whereas the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e. bottom) strand of the target nucleic acid. It can be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e. top) strand of the target nucleic acid. The PI domain, as its name suggests, contributes to PAM specificity.

While certain functions of Cas9 are linked to (but not necessarily fully determined by) the specific domains set forth above, these and other functions can be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains).

Cpf1

The crystal structure of Acidaminococcus sp. Cpf1 in complex with crRNA and a double-stranded (ds) DNA target including a TTTN PAM sequence has been solved by Yamano et al. (Cell. 2016 May 5; 165(4): 949-962 (Yamano), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

Modifications of RNA Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that can be made in the RuvC domains, in the Cas9 HNH domain, or in the Cpf1 Nuc domain are described in Ran and Yamano, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated.

Modifications of PAM specificity relative to naturally occurring Cas9 reference molecules has been described by Kleinstiver et al. for both S. pyogenes (Kleinstiver et al., Nature. 2015 Jul. 23; 523(7561):481-5 (Kleinstiver I) and S. aureus (Kleinstiver et al., Nat Biotechnol. 2015 December; 33(12): 1293-1298 (Klienstiver II)). Kleinstiver et al. have also described modifications that improve the targeting fidelity of Cas9 (Nature, 2016 Jan. 28; 529, 490-495 (Kleinstiver III)). Each of these references is incorporated by reference herein.

RNA-guided nucleases have been split into two or more parts, as described by Zetsche et al. (Nat Biotechnol. 2015 February; 33(2):139-42 (Zetsche II), incorporated by reference), and by Fine et al. (Sci Rep. 2015 Jul. 1; 5:10777 (Fine), incorporated by reference).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger et al., Nature Biotechnology 32, 577-582 (2014), which is incorporated by reference for all purposes herein.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications can be possible or desirable in certain applications. For brevity, therefore, exemplary systems, methods and compositions of the present disclosure are presented with reference to particular RNA-guided nucleases, but it should be understood that the RNA-guided nucleases used can be modified in ways that do not alter their operating principles. Such modifications are within the scope of the present disclosure.

### Nucleic Acids Encoding RNA-Guided Nucleases

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease can comprise a nuclear localization sequence (NLS). Nuclear localization sequences are known in the art.

### Functional Analysis of Candidate Molecules

Candidate RNA-guided nucleases, gRNAs, and complexes thereof, can be evaluated by standard methods known in the art. See, e.g. Cotta-Ramusino. The stability of RNP complexes can be evaluated by differential scanning fluorimetry, as described below.

Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g. different stoichiometric ratios of gRNA: RNA-guided nuclease protein, different buffer solutions, etc.) to identify optimal conditions for RNP formation; and (b) testing modifications (e.g. chemical modifications, alterations of sequence, etc.) of an RNA-guided nuclease and/or a gRNA to identify those modifications that improve RNP formation or stability. One readout of a DSF assay is a shift in melting temperature of the RNP complex; a relatively high shift suggests that the RNP complex is more stable (and can thus have greater activity or more favorable kinetics of formation, kinetics of degradation, or another functional characteristic) relative to a reference RNP complex characterized by a lower shift. When the DSF assay is deployed as a screening tool, a threshold melting temperature shift can be specified, so that the output is one or more RNPs having a melting temperature shift at or above the threshold. For instance, the threshold can be 5-10° C. (e.g. 5°, 6°, 7°, 8°, 9°, 10°) or more, and the output can be one or more RNPs characterized by a melting temperature shift greater than or equal to the threshold.

Two non-limiting examples of DSF assay conditions are set forth below:

To determine the best solution to form RNP complexes, a fixed concentration (e.g. 2 μM) of Cas9 in water+10× SYPRO Orange® (Life Technologies cat #S-6650) is dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with fixed concentration (e.g. 2 μM) Cas9 in optimal buffer from assay 1 above and incubating (e.g. at RT for 10′) in a 384 well plate. An equal volume of optimal buffer+10× SYPRO Orange® (Life Technologies cat #S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e. to alter) targeted regions of DNA within or obtained from a cell. Various strategies are described herein to generate particular edits, and these strategies are generally described in terms of the desired repair outcome, the number and positioning of individual edits (e.g. SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs can result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g. a homologous sequence within the cellular genome), to promote gene conversion. Exogenous templates can have asymmetric overhangs (i.e. the portion of the template that is complementary to the site of the DSB can be offset in a 3′ or 5′ direction, rather than being centered within the donor template), for instance as described by Richardson et al. (Nature Biotechnology 34, 339-344 (2016), (Richardson), incorporated by reference). In instances where the template is single stranded, it can correspond to either the complementary (top) or non-complementary (bottom) strand of the targeted region.

Gene conversion and gene correction are facilitated, in some cases, by the formation of one or more nicks in or around the targeted region, as described in Ran and Cotta-Ramusino. In some cases, a dual-nickase strategy is used to form two offset SSBs that, in turn, form a single DSB having an overhang (e.g. a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends can be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g. ±1, ±2, ±3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

Multiplex Strategies

While exemplary strategies discussed above have focused on repair outcomes mediated by single DSBs, genome editing systems according to this disclosure can also be employed to generate two or more DSBs, either in the same locus or in different loci. Strategies for editing that involve the formation of multiple DSBs, or SSBs, are described in, for instance, Cotta-Ramusino.

Donor Template Design

Donor template design is described in detail in the literature, for instance in Cotta-Ramusino. DNA oligomer donor templates (oligodeoxynucleotides or ODNs), which can be single stranded (ssODNs) or double-stranded (dsODNs), can be used to facilitate HDR-based repair of DSBs, and are particularly useful for introducing alterations into a target DNA sequence, inserting a new sequence into the target sequence, or replacing the target sequence altogether.

Whether single-stranded or double stranded, donor templates generally include regions that are homologous to regions of DNA within or near (e.g. flanking or adjoining) a target sequence to be cleaved. These homologous regions are referred to here as “homology arms,” and are illustrated schematically below:
[5′ homology arm]-[replacement sequence]-[3′ homology arm].

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In certain embodiments, both the 5′ and the 3′ homology arms can be shortened to avoid including certain sequence repeat elements. In addition, some homology arm designs can improve the efficiency of editing or increase the frequency of a desired repair outcome. For example, Richardson et al. Nature Biotechnology 34, 339-344 (2016) (Richardson), which is incorporated by reference, found that the relative asymmetry of 3′ and 5′ homology arms of single stranded donor templates influenced repair rates and/or outcomes.

Replacement sequences in donor templates have been described elsewhere, including in Cotta-Ramusino et al. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or code for, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN can have any suitable length, e.g., about, at least, or no more than 150-200 nucleotides (e.g., 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g. in an AAV or lentiviral genome) that includes certain genomic backbone elements (e.g. inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

Target Cells

Genome editing systems according to this disclosure can be used to manipulate or alter a cell, e.g., to edit or alter a target nucleic acid. The manipulating can occur, in various embodiments, in vivo or ex vivo.

A variety of cell types can be manipulated or altered according to the embodiments of this disclosure, and in some cases, such as in vivo applications, a plurality of cell types are altered or manipulated, for example by delivering genome editing systems according to this disclosure to a plurality of cell types. In other cases, however, it can be desirable to limit manipulation or alteration to a particular cell type or types. For instance, it can be desirable in some instances to edit a cell with limited differentiation potential or a terminally differentiated cell, such as a photoreceptor cell in the case of Maeder, in which modification of a genotype is expected to result in a change in cell phenotype. In other cases, however, it can be desirable to, multipotent or pluripotent, stem or progenitor cell. By way of example, the cell edit a less differentiated can be an embryonic stem cell, induced pluripotent stem cell (iPSC), hematopoietic stem/progenitor cell (HSPC), or other stem or progenitor cell type that differentiates into a cell type of relevance to a given application or indication.

As a corollary, the cell being altered or manipulated is, variously, a dividing cell or a non-dividing cell, depending on the cell type(s) being targeted and/or the desired editing outcome.

When cells are manipulated or altered ex vivo, the cells can be used (e.g. administered to a subject) immediately, or they can be maintained or stored for later use. Those of skill in the art will appreciate that cells can be maintained in culture or stored (e.g. frozen in liquid nitrogen) using any suitable method known in the art.

The compositions or systems described herein can be delivered to a target cell. In certain embodiments, the target cell is an epithelial cell, e.g., an epithelial cell of the oropharynx (including, e.g., an epithelial cell of the nose, gums, lips, tongue or pharynx), an epithelial cell of the finger or fingernail bed, or an epithelial cell of the ano-genital area (including, e.g., an epithelial cell of the penis, scrotum, vulva, vagina, cervix, anus or thighs). In certain embodiments, the target cell is a neuronal cell, e.g., a cranial ganglion neuron (e.g. a trigeminal ganglion neuron, e.g., an oculomotor nerve ganglion neuron, e.g., an abducens nerve ganglion neuron, e.g., a trochlear nerve ganglion neuron), e.g. a cervical ganglion neuron, e.g., a sacral ganglion neuron, a sensory ganglion neuron, a cortical neuron, a cerebellar neuron or a hippocampal neuron. In an embodiment, the target cell is an optic cell, e.g. an epithelial cell of the eye, e.g. an epithelial cell of the eyelid, e.g., a conjunctival cell, e.g., a conjunctival epithelial cell, e.g., a corneal keratocyte, e.g., a limbus cell, e.g., a corneal epithelial cell, e.g., a corneal endothelial cell, e.g., a corneal stromal cell, e.g., a ciliary body cell, e.g., a scleral cell, e.g., a lens cell, e.g., a choroidal cell, e.g., a retinal cell, e.g., a rod photoreceptor cell, e.g., a cone photoreceptor cell, e.g., a retinal pigment epithelium cell, e.g., a horizontal cell, e.g., an amacrine cell, e.g., a ganglion cell. In certain embodiments, the target cell can be a Muller cell, a bipolar cell; a ciliary muscle cell; a suspensory ligament cell; an iris muscle cell; a cell located on the Bruch's membrane; a trabecular meshwork cell; a zonule fiber cell; or any nerve cell that innervates the eye. In certain embodiments, the target cell is an optic cell, e.g. an endothelial cell of the eye, e.g., corneal endothelial cell, retinal vascular endothelial cell, e.g., choroidal endothelial cell, e.g., schlemn's canal endothelial cell, e.g., conjunctival endothelial cell, e.g., Lymphatic endothelial cell, e.g., a vascular smooth muscle cell., Iridial skeletal or smooth muscle cell, e.g., ciliary muscle cell, e.g., corneal dendritic cells. e.g., a suspensory ligament cell, e.g., trabecular meshwork cell, e.g., a zonule fiber cell, e.g., an iris pigment epithelium cell, e.g., lens epithelium, e.g., Lens fibre cell.

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. Tables 3 and 4 set forth several, non-limiting examples of genome editing system implementations. Those of skill in the art will appreciate, however, that these listings are not comprehensive, and that other implementations are possible. With reference to Table 3 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 3
Genome Editing System Components

| RNA-guided | | Donor | |
|---|---|---|---|
| Nuclease | gRNA | Template | Comments |
| Protein | RNA | [N/A] | An RNA-guided nuclease protein |
| | | | complexed with a gRNA molecule |
| | | | (an RNP complex) |
| Protein | RNA | DNA | An RNP complex as described |
| | | | above plus a single-stranded or |
| | | | double stranded donor template. |
| Protein | DNA | [N/A] | An RNA-guided nuclease protein |
| | | | plus gRNA transcribed from DNA. |
| Protein | DNA | DNA | An RNA-guided nuclease protein |
| | | | plus gRNA-encoding DNA and a |
| | | | separate DNA donor template. |
| Protein | DNA | | An RNA-guided nuclease protein |
| | | | and a single DNA encoding both a |
| | | | gRNA and a donor template. |
| DNA | | | A DNA or DNA vector encoding |
| | | | an RNA-guided nuclease, a gRNA |
| | | | and a donor template. |
| DNA | DNA | [N/A] | Two separate DNAs, or two |
| | | | separate DNA vectors, encoding |
| | | | the RNA-guided nuclease and the |
| | | | gRNA, respectively. |
| DNA | DNA | DNA | Three separate DNAs, or three |
| | | | separate DNA vectors, encoding |
| | | | the RNA-guided nuclease, the |
| | | | gRNA and the donor template, |
| | | | respectively. |
| DNA | | [N/A] | A DNA or DNA vector encoding |
| | | | an RNA-guided nuclease and a |
| | | | gRNA |
| DNA | | DNA | A first DNA or DNA vector |
| | | | encoding an RNA-guided nuclease |
| | | | and a gRNA, and a second DNA or |
| | | | DNA vector encoding a donor |
| | | | template. |
| DNA | DNA | | A first DNA or DNA vector |
| | | | encoding an RNA-guided nuclease |
| | | | and second DNA or DNA vector |
| | | | encoding a gRNA and a donor |
| | | | template. |
| DNA | | | A first DNA or DNA vector |
| DNA | | | encoding an RNA-guided nuclease |
| | | | and a donor template, and a second |
| | | | DNA or DNA vector encoding a |
| | | | gRNA |
| DNA | | | A DNA or DNA vector encoding |
| RNA | | | an RNA-guided nuclease and a |
| | | | donor template, and a gRNA |
| RNA | | [N/A] | An RNA or RNA vector encoding |
| | | | an RNA-guided nuclease and |
| | | | comprising a gRNA |
| RNA | | DNA | An RNA or RNA vector encoding |
| | | | an RNA-guided nuclease and |
| | | | comprising a gRNA, and a DNA or |
| | | | DNA vector encoding a donor |
| | | | template. |

Table 4 summarizes various delivery methods for the components of genome editing systems, as described herein. Again, the listing is intended to be exemplary rather than limiting.

TABLE 4

| | | Delivery | | | |
|---|---|---|---|---|---|
| | | into | Duration | | |
| | | Non- | of | | Type of |
| | | Dividing | Expres- | Genome | Molecule |
| Delivery Vector/Mode | | Cells | sion | Integration | Delivered |
| Physical (e.g., | | YES | Transient | NO | Nucleic Acids |
| electroporation, particle | | | | | and Proteins |
| gun, Calcium Phosphate | | | | | |
| transfection, cell | | | | | |
| compression | | | | | |
| or squeezing) | | | | | |
| Viral | Retrovirus | NO | Stable | YES | RNA |
| | Lentivirus | YES | Stable | YES/NO | RNA |
| | | | | with | |
| | | | | modifica- | |
| | | | | tions | |
| | Adenovirus | YES | Stable | NO | DNA |
| | Adeno- | YES | Stable | NO | DNA |
| | Associated | | | | |
| | Virus (AAV) | | | | |
| | Vaccinia | YES | Very | NO | DNA |
| | Virus | | Transient | | |
| | Herpes | YES | Stable | NO | DNA |
| | Simplex | | | | |
| | Virus | | | | |
| Non-Viral | Cationic | YES | Transient | Depends | Nucleic Acids |
| | Liposomes | | | on what is | and Proteins |
| | | | | delivered | |
| | Polymeric | YES | Transient | Depends | Nucleic Acids |
| | Nano- | | | on what is | and Proteins |
| | particles | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacterio- | | | | |
| | phages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes). Nucleic acid vectors, such as the vectors summarized in Table 4, can also be used.

Nucleic acid vectors can comprise one or more sequences encoding genome editing system components, such as an RNA-guided nuclease, a gRNA and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Exemplary viral vectors are set forth in Table 4, and additional suitable viral vectors and their use and production are described in Cotta-Ramusino. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in Cotta-Ramusino. Any suitable nanoparticle design can be used to deliver genome editing system components or nucleic acids encoding such components. For instance, organic (e.g. lipid and/or polymer) nonparticles can be suitable for use as delivery vehicles in certain embodiments of this disclosure. Exemplary lipids for use in nanoparticle formulations, and/or gene transfer are shown in Table 5, and Table 6 lists exemplary polymers for use in gene transfer and/or nanoparticle formulations.

TABLE 5
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)propyl]N,N,N-trimethylammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N-dimethyl- | DOSPA | Cationic |
| 1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

TABLE 6
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PANIAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In certain embodiments, the nucleic acid molecule is delivered at the same time as one or more of the components of the Genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Genome editing system are delivered. In certain embodiments, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

Delivery of RNPs and/or RNA Encoding Genome Editing System Components

RNPs (complexes of gRNAs and RNA-guided nucleases) and/or RNAs encoding RNA-guided nucleases and/or gRNAs, can be delivered into cells or administered to subjects by art-known methods, some of which are described in Cotta-Ramusino. In vitro, RNA-guided nuclease-encoding and/or gRNA-encoding RNA can be delivered, e.g., by microinjection, electroporation, transient cell compression or squeezing (see, e.g., Lee 2012). Lipid-mediated transfection, peptide-mediated delivery, GalNAc- or other conjugate-mediated delivery, and combinations thereof, can also be used for delivery in vitro and in vivo.

In vitro, delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

Route of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that can occur when therapeutically effective amounts of a component are administered systemically.

In certain embodiments, the genome editing systems disclosed herein are administered to a subject through direct trigeminal ganglion (TG) injection, intrastromal injection, subconjunctival injection, or corneal scarification. In certain embodiments, administration of the genome editing systems through intrastromal injection or direct TG injection effectively delivers the genome editing systems to a target tissue of the subject, e.g., corneas and/or TGs. In certain embodiments, administration of the genome editing systems through subconjunctival injection effectively delivers the genome systems to the target tissue of the subject, e.g., TGs. In certain embodiments, administration of the genome editing systems through corneal scarification effectively delivers the genome systems to the target tissue of the subject, e.g., corneas.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful; however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems can be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically, the microspheres are composed of a polymer of lactic acid and glycolic acid, which are structured to form hollow spheres. The spheres can be approximately 15-30 microns in diameter and can be loaded with components described herein.

Multi-Modal or Differential Delivery of Components

Skilled artisans will appreciate, in view of the instant disclosure, that different components of genome editing systems disclosed herein can be delivered together or separately and simultaneously or non-simultaneously. Separate and/or asynchronous delivery of genome editing system components can be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., an RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV, or lentivirus, delivery.

By way of example, the components of a genome editing system, e.g., an RNA-guided nuclease and a gRNA, can be delivered by modes that differ in terms of resulting half-life or persistent of the delivered component the body, or in a particular compartment, tissue or organ. In certain embodiments, a gRNA can be delivered by such modes. The RNA-guided nuclease molecule component can be delivered by a mode which results in less persistence or less exposure to the body or a particular compartment or tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV, or lentivirus vector. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV, or lentivirus vector. Transcription of these genes would be of little physiological consequence because the genes do not encode for a protein product, and the gRNAs are incapable of acting in isolation. The second component, an RNA-guided nuclease molecule, is delivered in a transient manner, for example as mRNA or as protein, ensuring that the full RNA-guided nuclease molecule/gRNA complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA is delivered by a first delivery mode that results in a first spatial, e.g., tissue, distribution. A second component, e.g., an RNA-guided nuclease molecule is delivered by a second delivery mode that results in a second spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. In certain embodiments, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it can be desirable to only target a single tissue. A two-part delivery system can resolve this challenge and enhance tissue specificity. If the gRNA and the RNA-guided nuclease molecule are packaged in separated delivery vehicles with distinct but overlapping tissue tropism, the fully functional complex is only being formed in the tissue that is targeted by both vectors.

### List of Exemplary Embodiments

1. A genome editing system comprising: (a) a first gRNA molecule comprising a first targeting domain that is complementary with a first target sequence of a first HSV-1 gene, (b) a second gRNA molecule comprising a second targeting domain that is complementary with a second target sequence of a second HSV-1 gene, and (c) an RNA-guided nuclease.
2. The genome editing system of 1, wherein the first HSV-1 gene is different from the second HSV-1 gene.
3. The genome editing system of 1, wherein the first HSV-1 gene is the same as the second HSV-1 gene.
4. The genome editing system of any one of 1-3, wherein each of the first and second HSV-1 genes is independently selected from the group consisting of immediate early HSV-1 genes, early HSV-1 genes, and late HSV-1 genes.
5. The genome editing system of 4, wherein the immediate-early HSV-1 genes are selected from the group consisting of a RL2 gene, a RS1 gene, a UL54 gene, a US1 gene, a US1.5 gene, and a US12 gene.
6. The genome editing system of 4 or 5, wherein the immediate-early HSV-1 genes are selected from the group consisting of a RL2 gene, a RS1 gene, and a UL54 gene.
7. The genome editing system of any one of 4-6, wherein the early HSV-1 genes are selected from the group consisting of a UL5 gene, a UL8 gene, a UL9 gene, a UL23 gene, a UL29 gene, a UL30 gene, a UL42 gene, and a UL52 gene.
8. The genome editing system of any one of 4-7, wherein the early HSV-1 gene is a UL29 gene.
9. The genome editing system of any one of 4-8, wherein the late HSV-1 genes are selected from the group consisting of a UL1 gene, a UL6 gene, a UL15 gene, a UL16 gene, a UL18 gene, a UL19 gene, a UL22 gene, a UL26 gene, a UL26.5 gene, a UL27 gene, a UL28 gene, a UL31 gene, a UL32 gene, a UL33 gene, a UL34 gene, a UL35 gene, a UL36 gene, a UL37 gene, a UL38 gene, a UL48 gene, a UL49.5 gene, and a US6 gene.
10. The genome editing system of any one of 4-9, wherein the late HSV-1 genes are selected from the group consisting of a UL6 gene, a UL15 gene, a UL19 gene, a UL22 gene, a UL32 gene, a UL33 gene, a UL37 gene, and a UL48 gene.
11. The genome editing system of any one of 1-10, wherein the first HSV-1 gene is a late HSV-1 gene, and the second HSV-1 gene is an immediate early HSV-1 gene, an early HSV-1 gene, or a late HSV-1 gene.
12. The genome editing system of any one of 1-11, wherein the first HSV-1 gene is a late HSV-1 gene, and the second HSV-1 gene is an immediate early HSV-1 gene.
13. The genome editing system of any one of 1-12, wherein the first HSV-1 gene is a UL48 gene, and the second HSV-1 gene is a RL2 gene.
14. The genome editing system of any one of 1-11, wherein the first HSV-1 gene is a late HSV-1 gene, and the second HSV-1 gene is a late HSV-1 gene.
15. The genome editing system of any one of 1-11, wherein the first HSV-1 gene is a late HSV-1 gene, and the second HSV-1 gene is an early HSV-1 gene.
16. The genome editing system of any one of 1-10, wherein the first HSV-1 gene is an early HSV-1 gene, and the second HSV-1 gene is an early HSV-1 gene.
17. The genome editing system of any one of 1-10, wherein the first HSV-1 gene is an early HSV-1 gene, and the second HSV-1 gene is an immediate early HSV-1 gene.
18. The genome editing system of any one of 1-10, wherein the first HSV-1 gene is an immediate early HSV-1 gene, and the second HSV-1 gene is an immediate early HSV-1 gene.
19. The genome editing system of any one of 1-18, wherein the RNA-guided nuclease is Cas9 and the first targeting domain comprises a nucleotide sequence selected from SEQ ID NOs: 1-411, and the second targeting domain comprises a nucleotide sequence selected from SEQ ID NOs: 1-411.
20. The genome editing system of any one of 1-19, wherein the RNA-guided nuclease is Cas9 and the first targeting domain comprises a nucleotide sequence selected from SEQ ID NOs: 1-54, 410, and 411, and the second targeting domain comprises a nucleotide sequence selected from SEQ ID NOs: 1-54, 410, and 411.
21. The genome editing system of any one of 1-20, wherein the RNA-guided nuclease is Cas9 and the first targeting domain comprises a nucleotide sequence selected from SEQ ID NOs: 1-25, 410, and 411, and the second targeting domain comprises a nucleotide sequence selected from SEQ ID NOs: 1-25, 410, and 411.
22. The genome editing system of any one of 1-21, wherein the RNA-guided nuclease is Cas9 and the first targeting domain comprises a nucleotide sequence selected from SEQ ID NOs: 1-14, 410, and 411, and the second targeting domain comprises a nucleotide sequence selected from SEQ ID NOs: 1-14, 410, and 411.
23. The genome editing system of any one of 1-22, wherein the RNA-guided nuclease is Cas9 and the first targeting domain comprises the nucleotide sequence set forth in SEQ ID NO: 410, and second targeting domain comprises the nucleotide sequence set forth in SEQ ID NO: 411.
24. The genome editing system of any one of 1-23, wherein the RNA-guided nuclease is a first Cas9 molecule, further comprising a second Cas9 molecule, both of which are configured to form complexes with the first and second gRNAs.
25. The genome editing system of 24, wherein at least one of the first and second Cas9 molecules comprises an S. pyogenes Cas9 molecule or an S. aureus Cas9 molecule.
26. The genome editing system of 24 or 25, wherein at least one of the first and second Cas9 molecules comprises a wild-type Cas9 molecule, a mutant Cas9 molecule, or a combination thereof.
27. The genome editing system of 26 wherein the mutant Cas9 molecule comprises a D10A mutation.
28. The genome editing system of any one of 1-27 further comprising a third gRNA molecule comprising a third targeting domain that is complementary with a third target sequence of a third HSV-1 gene.
29. The genome editing system of any one of 1-28 further comprising a fourth gRNA molecule comprising a fourth targeting domain that is complementary with a fourth target sequence of a fourth HSV-1 gene.
30. The genome editing system of any one of 1-29 further comprising a fifth gRNA molecule comprising a fifth targeting domain that is complementary with a fifth target sequence of a fifth HSV-1 gene.
31. A composition comprising: (a) a first gRNA molecule comprising a first targeting domain that is complementary with a first target sequence of a first HSV-1 gene, (b) a second gRNA molecule comprising a second targeting domain that is complementary with a second target sequence of a second HSV-1 gene, and (c) an RNA-guided nuclease.
32. The composition of 31, wherein the first HSV-1 gene is different from the second HSV-1 gene.
33. The composition of 31, wherein the first HSV-1 gene is the same as the second HSV-1 gene.
34. The composition of any one of 31-33, wherein each of the first and second HSV-1 genes is selected from the group consisting of immediate early HSV-1 genes, early HSV-1 genes, and late HSV-1 genes.
35. The composition of 33, wherein the immediate-early HSV-1 genes are selected from the group consisting of a RL2 gene, a RS1 gene, a UL54 gene, a US1 gene, a US1.5 gene, and a US12 gene.
36. The composition of 34 or 35, wherein the immediate-early HSV-1 genes are selected from the group consisting of a RL2 gene, a RS1 gene, and a UL54 gene.
37. The composition of any one of 34-36, wherein the early HSV-1 genes are selected from the group consisting of a UL5 gene, a UL8 gene, a UL9 gene, a UL23 gene, a UL29 gene, a UL30 gene, a UL42 gene, and a UL52 gene.
38. The composition of any one of 34-37, wherein the early HSV-1 gene is a UL29 gene.
39. The composition of any one of 35-38, wherein the late HSV-1 genes are selected from the group consisting of a UL1 gene, a UL6 gene, a UL15 gene, a UL16 gene, a UL18 gene, a UL19 gene, a UL22 gene, a UL26 gene, a UL26.5 gene, a UL27 gene, a UL28 gene, a UL31 gene, a UL32 gene, a UL33 gene, a UL34 gene, a UL35 gene, a UL36 gene, a UL37 gene, a UL38 gene, a UL48 gene, a UL49.5 gene, and a US6 gene.
40. The composition of any one of 35-39, wherein the late HSV-1 genes are selected from the group consisting of a UL6 gene, a UL15 gene, a UL19 gene, a UL22 gene, a UL32 gene, a UL33 gene, a UL37 gene, and a UL48 gene.
41. The composition of any one of 31-40, wherein the first HSV-1 gene is a late HSV-1 gene, and the second HSV-1 gene is an immediate early HSV-1 gene, an early HSV-1 gene, or a late HSV-1 gene.
42. The composition of any one of 31-41, wherein the first HSV-1 gene is a late HSV-1 gene, and the second HSV-1 gene is an immediate early HSV-1 gene.
43. The composition of any one of 31-42, wherein the first HSV-1 gene is a UL48 gene, and the second HSV-1 gene is a RL2 gene.
44. The composition of any one of 31-41, wherein the first HSV-1 gene is a late HSV-1 gene, and the second HSV-1 gene is a late HSV-1 gene.
45. The composition of any one of 31-41, wherein the first HSV-1 gene is a late HSV-1 gene, and the second HSV-1 gene is an early HSV-1 gene.
46. The composition of any one of 31-41, wherein the first HSV-1 gene is an early HSV-1 gene, and the second HSV-1 gene is an early HSV-1 gene.
47. The composition of any one of 31-41, wherein the first HSV-1 gene is an early HSV-1 gene, and the second HSV-1 gene is an immediate early HSV-1 gene.
48. The composition of any one of 31-41, wherein the first HSV-1 gene is an immediate early HSV-1 gene, and the second HSV-1 gene is an immediate early HSV-1 gene.
49. The composition of any one of 31-48, wherein the RNA-guided nuclease is Cas9 and the first targeting domain comprises a nucleotide sequence selected from SEQ ID NOs: 1-411, and the second targeting domain comprises a nucleotide sequence selected from SEQ ID NOs: 1-411.
50. The composition of any one of 31-49, wherein the RNA-guided nuclease is Cas9 and the first targeting domain comprises a nucleotide sequence selected from SEQ ID NOs: 1-54, 410, and 411, and the second targeting domain comprises a nucleotide sequence selected from SEQ ID NOs: 1-54, 410, and 411.
51. The composition of any one of 31-50, wherein the RNA-guided nuclease is Cas9 and the first targeting domain comprises a nucleotide sequence selected from SEQ ID NOs: 1-25, 410, and 411, and the second targeting domain comprises a nucleotide sequence selected from SEQ ID NOs: 1-25, 410, and 411.
52. The composition of any one of 31-51, wherein the RNA-guided nuclease is Cas9 and the first targeting domain comprises a nucleotide sequence selected from SEQ ID NOs: 1-14, 410, and 411, and the second targeting domain comprises a nucleotide sequence selected from SEQ ID NOs: 1-14, 410, and 411.
53. The composition of any one of 31-52, wherein the RNA-guided nuclease is Cas9 and the first targeting domain comprises the nucleotide sequence set forth in SEQ ID NO: 410, and second targeting domain comprises the nucleotide sequence set forth in SEQ ID NO: 411.
54. The composition of any one of 31-53, wherein the RNA-guided nuclease is a first Cas9 molecule and further comprising a second Cas9 molecule, both of which are configured to form complexes with the first and second gRNAs.
55. The composition of 54, wherein at least one of the first and second Cas9 molecules comprises an S. pyogenes Cas9 molecule or an S. aureus Cas9 molecule.
56. The composition of 54 or 55, wherein at least one of the first and second Cas9 molecules comprises a wild-type Cas9 molecule, a mutant Cas9 molecule, or a combination thereof.
57. The composition of 56, wherein the mutant Cas9 molecule comprises a D10A mutation.
58. The composition of any one of 31-57 further comprising a third gRNA molecule comprising a third targeting domain that is complementary with a third target sequence of a third HSV-1 gene.
59. The composition of any one of 31-58 further comprising a fourth gRNA molecule comprising a fourth targeting domain that is complementary with a fourth target sequence of a fourth HSV-1 gene.
60. The composition of any one of 31-59 further comprising a fifth gRNA molecule comprising a fifth targeting domain that is complementary with a fifth target sequence of a fifth HSV-1 gene.
61. A vector comprising a polynucleotide encoding (a) a first gRNA molecule comprising a first targeting domain that is complementary with a first target sequence of a first HSV-1 gene, (b) a second gRNA molecule comprising a second targeting domain that is complementary with a second target sequence of a second HSV-1 gene, and (c) an RNA-guided nuclease.
62. The vector of 61, wherein the first HSV-1 gene is different from the second HSV-1 gene.
63. The vector of 61, wherein the first HSV-1 gene is the same as the second HSV-1 gene.
64. The vector of any one of 61-63, wherein each of the first and second HSV-1 genes is selected from the group consisting of immediate early HSV-1 genes, early HSV-1 genes, and late HSV-1 genes.
65. The vector of 64, wherein the immediate-early HSV-1 genes are selected from the group consisting of a RL2 gene, a RS1 gene, a UL54 gene, a US1 gene, a US1.5 gene, and a US12 gene.
66. The vector of 64 or 65, wherein the immediate-early HSV-1 genes are selected from the group consisting of a RL2 gene, a RS1 gene, and a UL54 gene.
67. The vector of any one of 64-66, wherein the early HSV-1 genes are selected from the group consisting of a UL5 gene, a UL8 gene, a UL9 gene, a UL23 gene, a UL29 gene, a UL30 gene, a UL42 gene, and a UL52 gene.
68. The vector of any one of 64-67, wherein the early HSV-1 gene is a UL29 gene.
69. The vector of any one of 64-68, wherein the late HSV-1 genes are selected from the group consisting of a UL1 gene, a UL6 gene, a UL15 gene, a UL16 gene, a UL18 gene, a UL19 gene, a UL22 gene, a UL26 gene, a UL26.5 gene, a UL27 gene, a UL28 gene, a UL31 gene, a UL32 gene, a UL33 gene, a UL34 gene, a UL35 gene, a UL36 gene, a UL37 gene, a UL38 gene, a UL48 gene, a UL49.5 gene, and a US6 gene.
70. The vector of any one of 64-69, wherein the late HSV-1 genes are selected from the group consisting of a UL6 gene, a UL15 gene, a UL19 gene, a UL22 gene, a UL32 gene, a UL33 gene, a UL37 gene, and a UL48 gene.
71. The vector of any one of 61-70, wherein the first HSV-1 gene is a late HSV-1 gene, and the second HSV-1 gene is an immediate early HSV-1 gene, an early HSV-1 gene, or a late HSV-1 gene.
72. The vector of any one of 61-71, wherein the first HSV-1 gene is a late HSV-1 gene, and the second HSV-1 gene is an immediate early HSV-1 gene.
73. The vector of any one of 61-72, wherein the first HSV-1 gene is a UL48 gene, and the second HSV-1 gene is a RL2 gene.
74. The vector of any one of 61-71, wherein the first HSV-1 gene is a late HSV-1 gene, and the second HSV-1 gene is a late HSV-1 gene.
75. The vector of any one of 61-71, wherein the first HSV-1 gene is a late HSV-1 gene, and the second HSV-1 gene is an early HSV-1 gene.
76. The vector of any one of 61-70, wherein the first HSV-1 gene is an early HSV-1 gene, and the second HSV-1 gene is an early HSV-1 gene.
77. The vector of any one of 61-70, wherein the first HSV-1 gene is an early HSV-1 gene, and the second HSV-1 gene is an immediate early HSV-1 gene.
78. The vector of any one of 61-70, wherein the first HSV-1 gene is an immediate early HSV-1 gene, and the second HSV-1 gene is an immediate early HSV-1 gene.
79. The vector of any one of 61-78, wherein the RNA-guided nuclease is Cas9 and the first targeting domain comprises a nucleotide sequence selected from SEQ ID NOs: 1-411, and the second targeting domain comprises a nucleotide sequence selected from SEQ ID NOs: 1-411.
80. The vector of any one of 61-79, wherein the RNA-guided nuclease is Cas9 and the first targeting domain comprises a nucleotide sequence selected from SEQ ID NOs: 1-54, 410, and 411, and the second targeting domain comprises a nucleotide sequence selected from SEQ ID NOs: 1-54, 410, and 411.
81. The vector of any one of 61-80, wherein the RNA-guided nuclease is Cas9 and the first targeting domain comprises a nucleotide sequence selected from SEQ ID NOs: 1-25, 410, and 411, and the second targeting domain comprises a nucleotide sequence selected from SEQ ID NOs: 1-25, 410, and 411.
82. The vector of any one of 61-81, wherein the RNA-guided nuclease is Cas9 and the first targeting domain comprises a nucleotide sequence selected from SEQ ID NOs: 1-14, 410, and 411, and the second targeting domain comprises a nucleotide sequence selected from SEQ ID NOs: 1-14, 410, and 411.
83. The vector of any one of 61-82, wherein the RNA-guided nuclease is Cas9 and the first targeting domain comprises the nucleotide sequence set forth in SEQ ID NO: 410, and second targeting domain comprises the nucleotide sequence set forth in SEQ ID NO: 411.
84. The vector of any one of 61-83, wherein the RNA-guided nuclease is a first Cas9 molecule and further comprising a second Cas9 molecule, both of which are configured to form complexes with the first and second gRNAs.
85. The vector of 84, wherein at least one of the first and second Cas9 molecules comprises an S. pyogenes Cas9 molecule or an S. aureus Cas9 molecule.
86. The vector of 84 or 85, wherein at least one of the first and second Cas9 molecules comprises a wild-type Cas9 molecule, a mutant Cas9 molecule, or a combination thereof.
87. The vector of 86, wherein the mutant Cas9 molecule comprises a D10A mutation.
88. The vector of any one of 61-87, wherein, the vector is a viral vector.
89. The vector of any one of 61-88, wherein the vector is an Adeno-associated virus (AAV) vector.
90. The vector of 89, wherein the AAV vector is a serotype 1, 2, 3, 4, 5, 6, 7, 8 or 9 vector.
91. The vector of any one of 61-90, further comprising a third gRNA molecule comprising a third targeting domain that is complementary with a third target sequence of a third HSV-1 gene.
92. The vector of any one of 61-91, further comprising a fourth gRNA molecule comprising a fourth targeting domain that is complementary with a fourth target sequence of a fourth HSV-1 gene.
93. The vector of any one of 59-92, further comprising a fifth gRNA molecule comprising a fifth targeting domain that is complementary with a fifth target sequence of a fifth HSV-1 gene.
94. A method of altering a first HSV-1 gene and a second HSV-1 gene in a cell, comprising administrating to the cell one of:

(i) a genome editing system comprising a first gRNA molecule comprising a first targeting domain that is complementary with a first target sequence of the first HSV-1 gene, a second gRNA molecule comprising a second targeting domain that is complementary with a second target sequence of the second HSV-1 gene, and at least an RNA-guided nuclease;

(ii) a genome editing system comprising a first polynucleotide encoding a first gRNA molecule comprising a first targeting domain that is complementary with a first target sequence of the first HSV-1 gene, a second polynucleotide encoding a second gRNA molecule comprising a second targeting domain that is complementary with a second target sequence of the second HSV-1 gene, and a third polynucleotide encoding an RNA-guided nuclease;

(iii) a composition comprising a first gRNA molecule comprising a first targeting domain that is complementary with a first target sequence of the first HSV-1 gene, a second gRNA molecule comprising a second targeting domain that is complementary with a target sequence of second HSV-1 gene, and at least an RNA-guided nuclease; and

(iv) a vector comprising a polynucleotide encoding (a) a first gRNA molecule comprising a first targeting domain that is complementary with a first target sequence of a first HSV-1 gene, (b) a second gRNA molecule comprising a second targeting domain that is complementary with a second target sequence of a second HSV-1 gene, and (c) at least an RNA-guided nuclease.

95. A method for treating or preventing a HSV-related disease in a subject, comprising administrating to the subject one of:

(i) a genome editing system comprising a first gRNA molecule comprising a first targeting domain that is complementary with a first target sequence of the first HSV-1 gene, a second gRNA molecule comprising a second targeting domain that is complementary with a second target sequence of the second HSV-1 gene, and at least an RNA-guided nuclease;

(ii) a genome editing system comprising a polynucleotide encoding a first gRNA molecule comprising a first targeting domain that is complementary with a first target sequence of the first HSV-1 gene, a polynucleotide encoding a second gRNA molecule comprising a second targeting domain that is complementary with a second target sequence of the second HSV-1 gene, and a polynucleotide encoding an RNA-guided nuclease;

(iii) a composition comprising a first gRNA molecule comprising a first targeting domain that is complementary with a first target sequence of the first HSV-1 gene, a second gRNA molecule comprising a second targeting domain that is complementary with a target sequence of second HSV-1 gene, and at least an RNA-guided nuclease; and

(iv) a vector comprising a polynucleotide encoding (a) a first gRNA molecule comprising a first targeting domain that is complementary with a first target sequence of a first HSV-1 gene, (b) a second gRNA molecule comprising a second targeting domain that is complementary with a second target sequence of a second HSV-1 gene, and (c) at least an RNA-guided nuclease.

96. The method of 95, wherein the HSV-related disease is a recurrent HSV-1 ocular keratitis.
97. The method of 95, wherein the HSV-related disease is a recurrent HSV-2 ocular keratitis.
98. The method of any one of 94-97, wherein the first HSV-1 gene is different from the second HSV-1 gene.
99. The method of any one of 94-97, wherein the first HSV-1 gene is the same as the second HSV-1 gene.
100. The method of any one of 94-99, wherein each of the first and second HSV-1 genes is selected from the group consisting of immediate early HSV-1 genes, early HSV-1 genes, and late HSV-1 genes.
101. The method of 100, wherein the immediate-early HSV-1 genes are selected from the group consisting of a RL2 gene, a RS1 gene, a UL54 gene, a US1 gene, a US1.5 gene, and a US12 gene.
102. The method of 100 or 101, wherein the immediate-early HSV-1 genes are selected from the group consisting of a RL2 gene, a RS1 gene, and a UL54 gene.
103. The method of any one of 100-102, wherein the early HSV-1 genes are selected from the group consisting of a UL5 gene, a UL8 gene, a UL9 gene, a UL23 gene, a UL29 gene, a UL30 gene, a UL42 gene, and a UL52 gene.
104. The method of any one of 100-103, wherein the early HSV-1 gene is a UL29 gene.
105. The method of any one of 100-104, wherein the late HSV-1 genes are selected from the group consisting of a UL1 gene, a UL6 gene, a UL15 gene, a UL16 gene, a UL18 gene, a UL19 gene, a UL22 gene, a UL26 gene, a UL26.5 gene, a UL27 gene, a UL28 gene, a UL31 gene, a UL32 gene, a UL33 gene, a UL34 gene, a UL35 gene, a UL36 gene, a UL37 gene, a UL38 gene, a UL48 gene, a UL49.5 gene, and a US6 gene.
106. The method of any one of 100-105, wherein the late HSV-1 genes are selected from the group consisting of a UL6 gene, a UL15 gene, a UL19 gene, a UL22 gene, a UL32 gene, a UL33 gene, a UL37 gene, and a UL48 gene.
107. The method of any one of 100-106, wherein the first HSV-1 gene is a late HSV-1 gene, and the second HSV-1 gene is an immediate early HSV-1 gene, an early HSV-1 gene, or a late HSV-1 gene.
108. The method of any one of 100-106, wherein the first HSV-1 gene is a late HSV-1 gene, and the second HSV-1 gene is an immediate early HSV-1 gene.
109. The method of any one of 100-108, wherein the first HSV-1 gene is a UL48 gene, and the second HSV-1 gene is a RL2 gene.
110. The method of any one of 100-107, wherein the first HSV-1 gene is a late HSV-1 gene, and the second HSV-1 gene is a late HSV-1 gene.
111. The method of any one of 100-107, wherein the first HSV-1 gene is a late HSV-1 gene, and the second HSV-1 gene is an early HSV-1 gene.
112. The method of any one of 100-106, wherein the first HSV-1 gene is an early HSV-1 gene, and the second HSV-1 gene is an early HSV-1 gene.
113. The method of any one of 100-106, wherein the first HSV-1 gene is an early HSV-1 gene, and the second HSV-1 gene is an immediate early HSV-1 gene.
114. The method of any one of 100-106, wherein the first HSV-1 gene is an immediate early HSV-1 gene, and the second HSV-1 gene is an immediate early HSV-1 gene.
115. The method of any one of 100-114, wherein the RNA-guided nuclease is a Cas9 molecule.
116. The method of any one of 100-115, wherein the first targeting domain comprises a nucleotide sequence selected from SEQ ID NOs: 1-411, and the second targeting domain comprises a nucleotide sequence selected from SEQ ID NOs: 1-411.
117. The method of any one of 100-116, wherein the RNA-guided nuclease is Cas9 and the first targeting domain comprises a nucleotide sequence selected from SEQ ID NOs: 1-54, 410, and 411, and the second targeting domain comprises a nucleotide sequence selected from SEQ ID NOs: 1-54, 410, and 411.
118. The method of any one of 100-117, wherein the RNA-guided nuclease is Cas9 and the first targeting domain comprises a nucleotide sequence selected from SEQ ID NOs: 1-25, 410, and 411, and the second targeting domain comprises a nucleotide sequence selected from SEQ ID NOs: 1-25, 410, and 411.
119. The method of any one of 100-118, wherein the RNA-guided nuclease is Cas9 and the first targeting domain comprises a nucleotide sequence selected from SEQ ID NOs: 1-14, 410, and 411, and the second targeting domain comprises a nucleotide sequence selected from SEQ ID NOs: 1-14, 410, and 411.
120. The method of any one of 100-119, wherein the first targeting domain comprises the nucleotide sequence set forth in SEQ ID NO: 410, and the second targeting domain comprises the nucleotide sequence set forth in SEQ ID NO: 411.
121. The method of any one of 100-120, wherein the subject is a human subject.
122. The method of any one of 100-121, wherein the administration is initiated prior to the subject having been exposed to a virus.
123. The method of any one of 100-122, wherein the administration is initiated prior to the HSV-related disease onset.
124. The method of any one of 100-123, wherein the administration is initiated in an advanced stage of the HSV-related disease.
125. The method of any one of 100-124, wherein the administration is initiated in an early stage of the HSV-related disease.
126. A method of administering a genome editing system to a subject, comprising administrating to the subject one of:

(i) a genome editing system comprising a first gRNA molecule comprising a first targeting domain that is complementary with a first target sequence of the first HSV-1 gene, a second gRNA molecule comprising a second targeting domain that is complementary with a second target sequence of the second HSV-1 gene, and at least an RNA-guided nuclease;

(ii) a genome editing system comprising a polynucleotide encoding a first gRNA molecule comprising a first targeting domain that is complementary with a first target sequence of the first HSV-1 gene, a polynucleotide encoding a second gRNA molecule comprising a second targeting domain that is complementary with a second target sequence of the second HSV-1 gene, and a polynucleotide encoding an RNA-guided nuclease;

(iii) a composition comprising a first gRNA molecule comprising a first targeting domain that is complementary with a first target sequence of the first HSV-1 gene, a second gRNA molecule comprising a second targeting domain that is complementary with a target sequence of second HSV-1 gene, and at least an RNA-guided nuclease; and

(iv) a vector comprising a polynucleotide encoding (a) a first gRNA molecule comprising a first targeting domain that is complementary with a first target sequence of a first HSV-1 gene, (b) a second gRNA molecule comprising a second targeting domain that is complementary with a second target sequence of a second HSV-1 gene, and (c) at least an RNA-guided nuclease.

127. The method of 126, wherein the HSV-related disease is a recurrent HSV-1 ocular keratitis.
128. The method of 126, wherein the HSV-related disease is a recurrent HSV-2 ocular keratitis.
129. The method of any one of 126-128, wherein the first HSV-1 gene is different from the second HSV-1 gene.
130. The method of any one of 126-128, wherein the first HSV-1 gene is the same as the second HSV-1 gene.
131. The method of any one of 126-130, wherein each of the first and second HSV-1 genes is selected from the group consisting of immediate early HSV-1 genes, early HSV-1 genes, and late HSV-1 genes.
132. The method of 131, wherein the immediate-early HSV-1 genes are selected from the group consisting of a RL2 gene, a RS1 gene, a UL54 gene, a US1 gene, a US1.5 gene, and a US12 gene.
133. The method of 131 or 132, wherein the immediate-early HSV-1 genes are selected from the group consisting of a RL2 gene, a RS1 gene, and a UL54 gene.
134. The method of any one of 131-133, wherein the early HSV-1 genes are selected from the group consisting of a UL5 gene, a UL8 gene, a UL9 gene, a UL23 gene, a UL29 gene, a UL30 gene, a UL42 gene, and a UL52 gene.
135. The method of any one of 131-134, wherein the early HSV-1 gene is a UL29 gene.
136. The method of any one of 131-135, wherein the late HSV-1 genes are selected from the group consisting of a UL1 gene, a UL6 gene, a UL15 gene, a UL16 gene, a UL18 gene, a UL19 gene, a UL22 gene, a UL26 gene, a UL26.5 gene, a UL27 gene, a UL28 gene, a UL31 gene, a UL32 gene, a UL33 gene, a UL34 gene, a UL35 gene, a UL36 gene, a UL37 gene, a UL38 gene, a UL48 gene, a UL49.5 gene, and a US6 gene.
137. The method of any one of 131-136, wherein the late HSV-1 genes are selected from the group consisting of a UL6 gene, a UL15 gene, a UL19 gene, a UL22 gene, a UL32 gene, a UL33 gene, a UL37 gene, and a UL48 gene.
138. The method of any one of 131-137, wherein the first HSV-1 gene is a late HSV-1 gene, and the second HSV-1 gene is an immediate early HSV-1 gene, an early HSV-1 gene, or a late HSV-1 gene.
139. The method of any one of 131-138, wherein the first HSV-1 gene is a late HSV-1 gene, and the second HSV-1 gene is an immediate early HSV-1 gene.
140. The method of any one of 131-139, wherein the first HSV-1 gene is a UL48 gene, and the second HSV-1 gene is a RL2 gene.
141. The method of any one of 131-138, wherein the first HSV-1 gene is a late HSV-1 gene, and the second HSV-1 gene is a late HSV-1 gene.
142. The method of any one of 131-138, wherein the first HSV-1 gene is a late HSV-1 gene, and the second HSV-1 gene is an early HSV-1 gene.
143. The method of any one of 131-137, wherein the first HSV-1 gene is an early HSV-1 gene, and the second HSV-1 gene is an early HSV-1 gene.
144. The method of any one of 131-137, wherein the first HSV-1 gene is an early HSV-1 gene, and the second HSV-1 gene is an immediate early HSV-1 gene.
145. The method of any one of 131-137, wherein the first HSV-1 gene is an immediate early HSV-1 gene, and the second HSV-1 gene is an immediate early HSV-1 gene.
146. The method of any one of 131-145, wherein the RNA-guided nuclease is a Cas9 molecule.
147. The method of any one of 131-146, wherein the first targeting domain comprises a nucleotide sequence selected from SEQ ID NOs: 1-411, and the second targeting domain comprises a nucleotide sequence selected from SEQ ID NOs: 1-411.
148. The method of any one of 131-147, wherein the RNA-guided nuclease is Cas9 and the first targeting domain comprises a nucleotide sequence selected from SEQ ID NOs: 1-54, 410, and 411, and the second targeting domain comprises a nucleotide sequence selected from SEQ ID NOs: 1-54, 410, and 411.
149. The method of any one of 131-148, wherein the RNA-guided nuclease is Cas9 and the first targeting domain comprises a nucleotide sequence selected from SEQ ID NOs: 1-25, 410, and 411, and the second targeting domain comprises a nucleotide sequence selected from SEQ ID NOs: 1-25, 410, and 411.
150. The method of any one of 131-149, wherein the RNA-guided nuclease is Cas9 and the first targeting domain comprises a nucleotide sequence selected from SEQ ID NOs: 1-14, 410, and 411, and the second targeting domain comprises a nucleotide sequence selected from SEQ ID NOs: 1-14, 410, and 411.
151. The method of any one of 131-150, wherein the first targeting domain comprises the nucleotide sequence set forth in SEQ ID NO: 410, and the second targeting domain comprises the nucleotide sequence set forth in SEQ ID NO: 411.
152. The method of any one of 131-151, wherein the subject is a human subject.
153. The method of any one of 131-152, wherein the administration is initiated prior to the subject having been exposed to a virus.
154. The method of any one of 131-153, wherein the administration is initiated prior to the HSV-related disease onset.
155. The method of any one of 131-154, wherein the administration is initiated in an advanced stage of the HSV-related disease.
156. The method of any one of 131-155, wherein the administration is initiated in an early stage of the HSV-related disease.

### EXAMPLES

The following Examples are merely illustrative and are not intended to limit the scope or content of the invention in any way.

### Example 1: Assessment of Gene Editing Approach Targeting Latent HSV-1 Genomes in Rabbit Model of Recurrent HSV-1 Keratitis

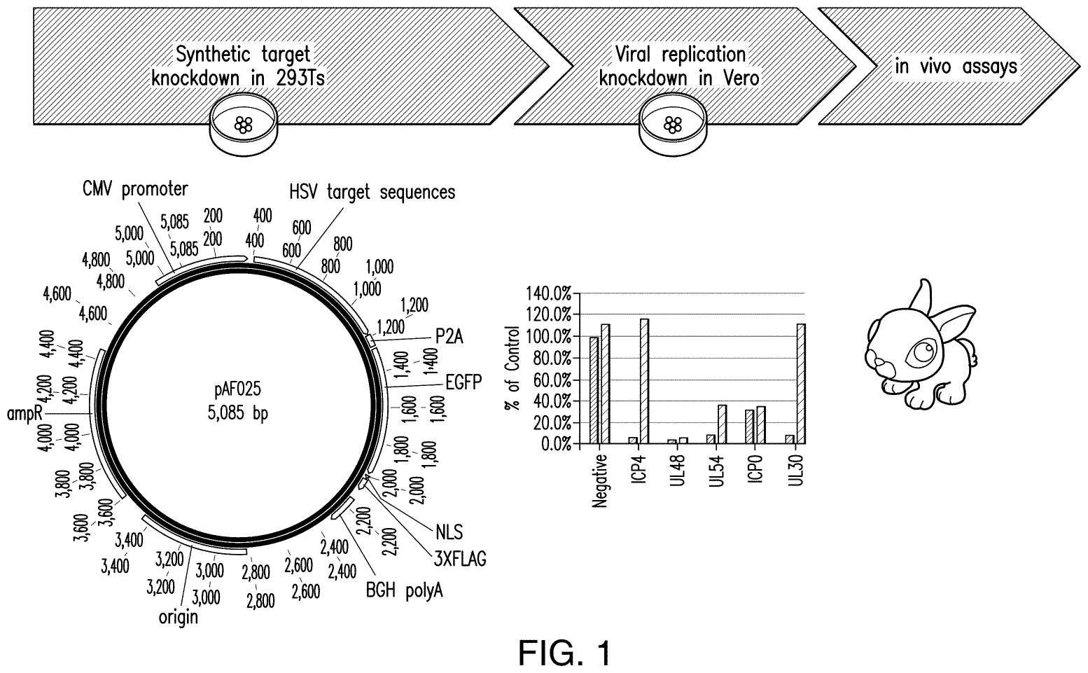

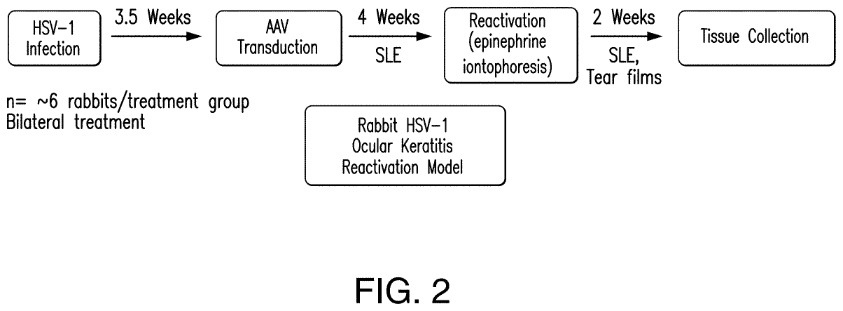

An adeno-associated virus (AAV) expressing either a RL2-specific gRNA having the nucleotide sequence set forth in SEQ ID NO: 410, or a UL48-specific gRNAs having the nucleotide sequence set forth in SEQ ID NO: 411 were tested to assess the efficacy of editing an immediate-early gene in combination with a late gene. The two gRNA sequences were packaged in AAV vector expressing CMV-driven Staph. aureus Cas9 (SaCas9) for the subsequent study. An efficacy study was conducted in the HSV-1 reactivation rabbit model to evaluate the potency of the CRISPR/Cas9 system with the selected gRNAs. As depicted in FIGS. 1 & 2, HSV-1 strain 17Syn+ was applied topically to scarified corneas of New Zealand white rabbit post-corneal abrasion and allowed to establish latency in the TG over four weeks. Rabbits were then treated with AAV vectors expressing Cas9 and gRNAs targeting GFP (negative control), or either UL48 and/or RL2. The vectors were applied, individually or in combination, to re-scarified corneas at a total of 1.0E+11 viral genomes/eye.

Latent HSV-1 was subsequently reactivated in each rabbit using epinephrine iontophoresis. After the reactivation, tear swabs were collected daily and slit lamp examinations (SLE) were conducted every 2-3 days. HSV-1 virions were detected in tear films using a plaque assay. Viral genomes in TG were quantified using qPCR. Statistical analyses were conducted using one-way ANOVA.

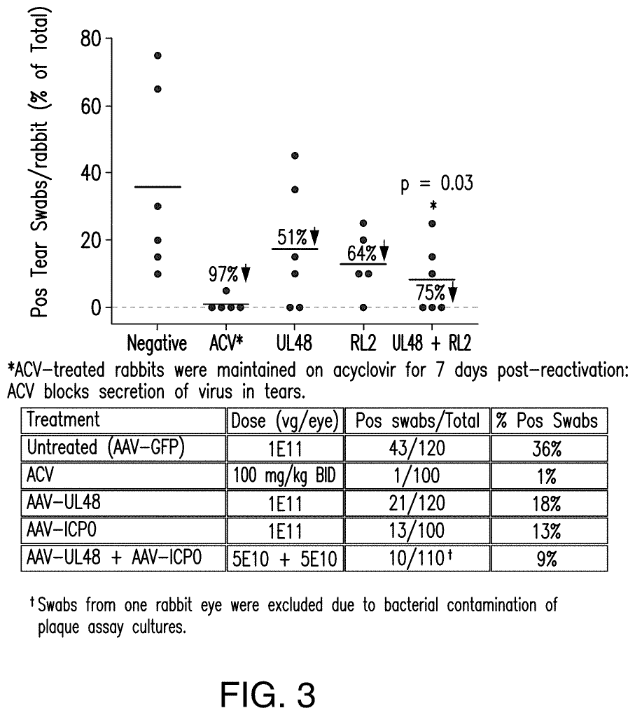

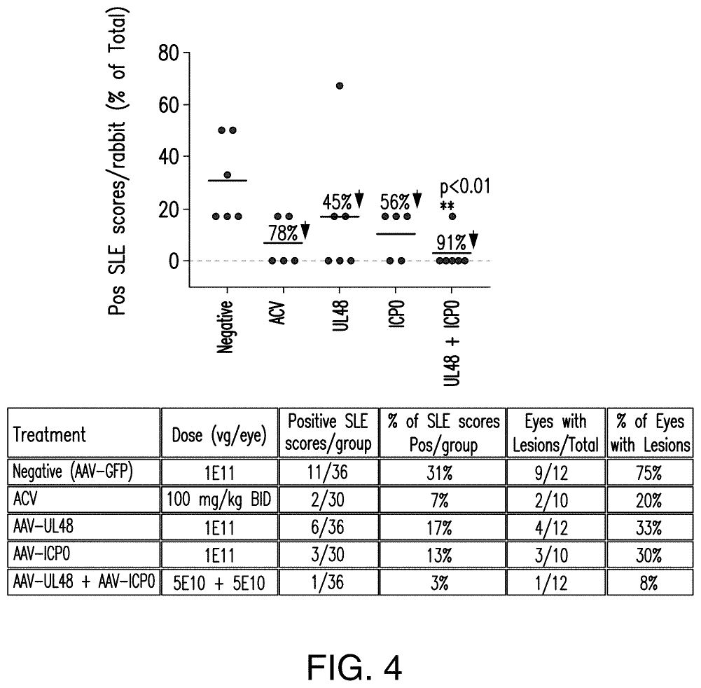

The combination of gRNA-expressing AAVs significantly reduced viral genomes produced in tears, as well as corneal lesions, at levels greater than the individual gRNAs alone. In particular, after reactivation, gRNAs targeting UL48 or RL2 inhibited HSV-1 production in tear films up to about 64% (FIG. 3, p>0.05) and corneal lesions were suppressed by up to about 56% (FIG. 4, p>0.05), comparing to the control group. A combination of gRNAs targeting UL48 and RL2 inhibited HSV-1 virions in tear films by about 75% (p=0.03) and reduced corneal lesions by about 91% (p<0.01), comparing to the control group (FIGS. 3 & 4). One-way ANOVA was used for statistical analysis.

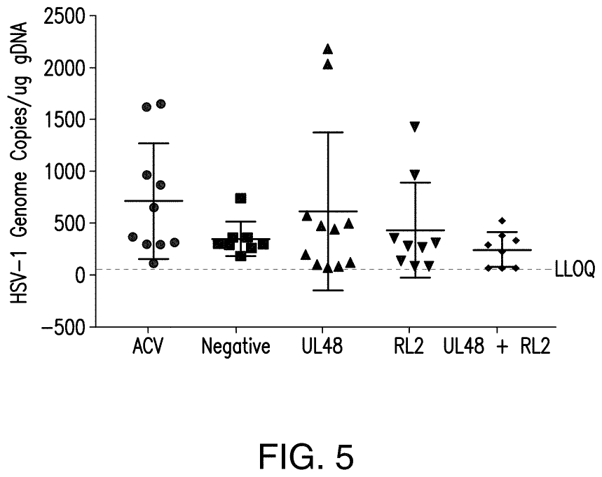

HSV-1 copy numbers in the TG collected from the reactivated rabbits were quantified by qPCR (FIG. 5). The results showed that HSV-1 copy number per μg rabbit gDNA was detected but low, and there was no significant reduction in HSV-1 copy number per TG comparing to negative control. In addition, viral genome editing and NHEJ repair is assessed by Illumina sequencing. The vector bio-distribution, Cas9/gRNA expression profiles, and HSV viral load in both corneal and TG tissues in rabbit, as well as in murine models, are being explored to further optimize gene editing and establish the PK/PD relationship of the drug candidates.

### Example 2: Selection and Evaluation of Candidate Guide RNAs (gRNAs)

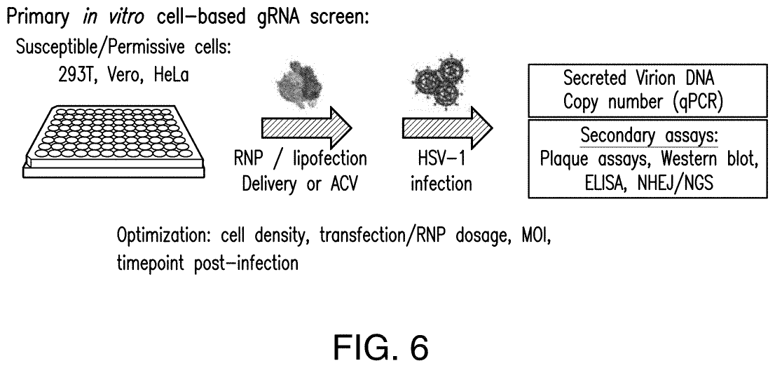

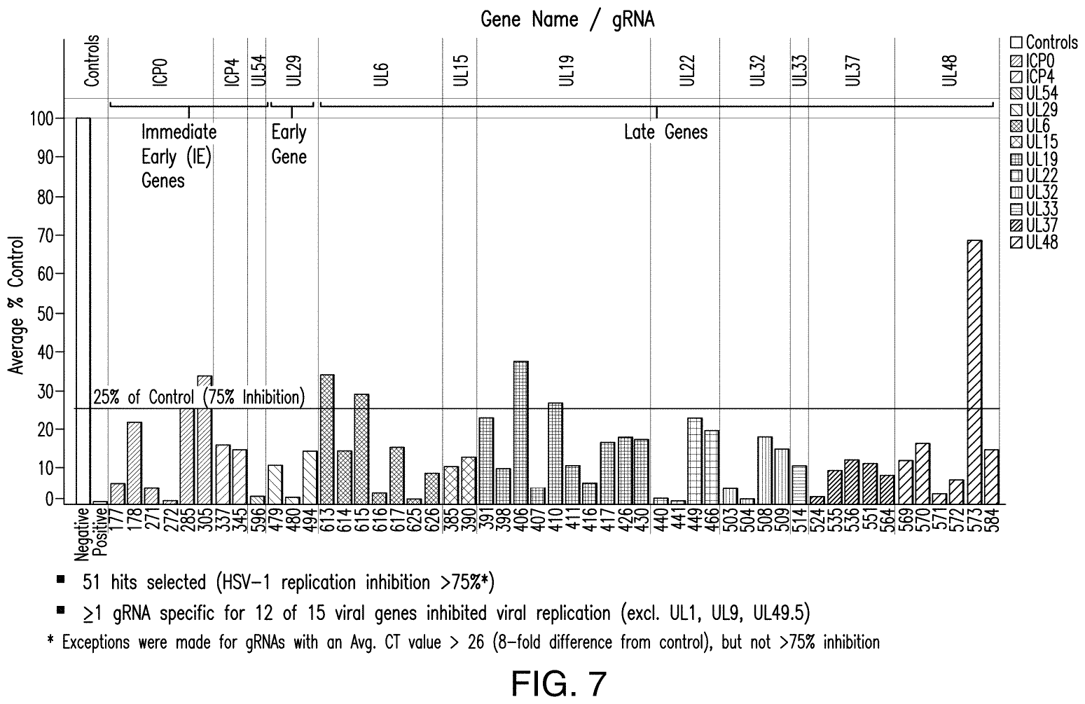

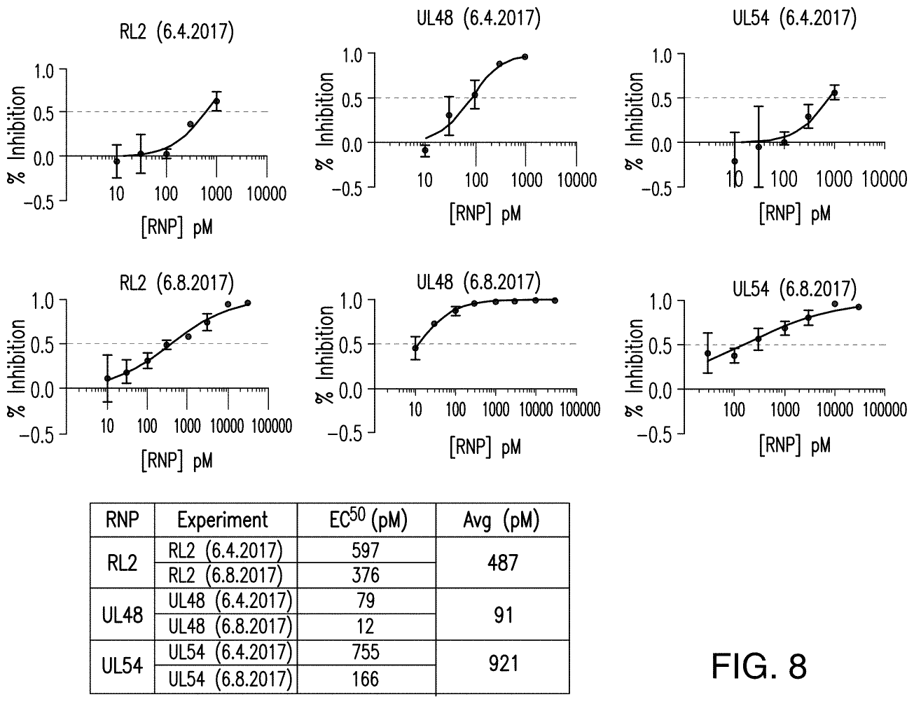

Candidate gRNAs (N=406) targeting 15 essential viral genes of HSV-1 were initially selected and generated based on predetermined criteria. Candidate gRNAs (N=406) were then assembled into ribonucleoproteins (RNPs) and screened against HSV-1-specific sequences in high throughput in vitro assays measuring reduction in the production of virus (FIG. 6). In particular, RNPs were complexed for each gRNA and tested for their ability to inhibit viral replication, as measured by production of virions from the infected cells. The production of virions was measured by secreted virion DNA copy numbers using qPCR. Secondary assays, including plaque assay, western blot, ELISA, NHEJ/NGS, were also used to measure the production of virions. Cell density, transfection/RNP dosage, MOI, and timepoint post-infection were optimized for the in vitro cell-based gRNA screen. Of the 406 gRNAs screened, 51 gRNAs targeting 12 of 15 essential viral genes were demonstrated to inhibit virion replication more than 75% (FIG. 7). Exceptions were made for gRNAs with an Average Ct value >26 (8-fold difference from control), but not >75% inhibition. EC₅₀ of RNP targeting RL2, UL48 or UL54 were also measured (FIG. 8).

Efficacy and potency of the selected 51 gRNAs is further confirmed and assessed followed by a combination screen. Combinations of potent gRNAs is assessed using the following criteria: 1) gRNAs targeting HSV-1 sequences within 500 nucleotides of each other; 2) gRNAs targeting different temporally expressed classes of viral genes; 3) gRNAs targeting different genes within the same temporally expressed class; 4) potent gRNA combinations from criterion 1 is assessed according to criteria 2 and 3.

These approaches can be expanded to all herpes viruses, including but not limited to CMV, KSHV, VZV, EBV, or HSV-2.

### Example 3: Selection of Lead gRNAs

A screening funnel was designed for identifying lead gRNAs using a number of techniques. Candidate gRNAs (N=406) targeting 15 essential viral genes of HSV-1 were initially selected and generated based on predetermined criteria. The targeting domains of the 406 gRNAs are set forth in SEQ ID NOs: 1-12, 14-23, and 25-408. The 406 candidate gRNAs were then assembled into ribonucleoproteins (RNPs) and screened against HSV-1-specific sequences in high throughput in vitro assays measuring reduction in the production of virus, as disclosed in Example 2.

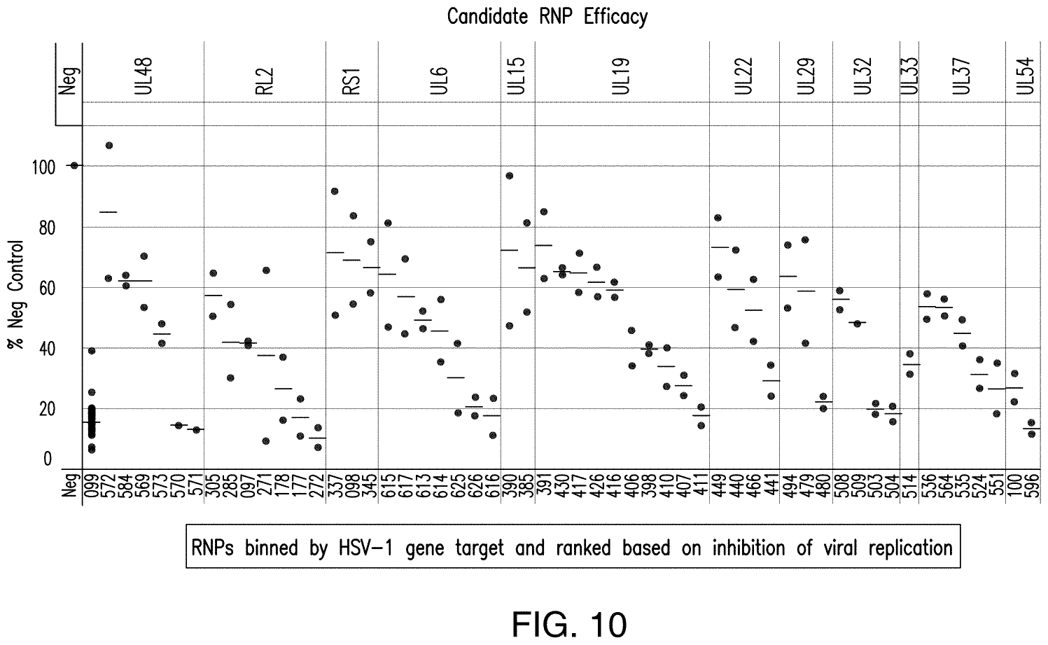

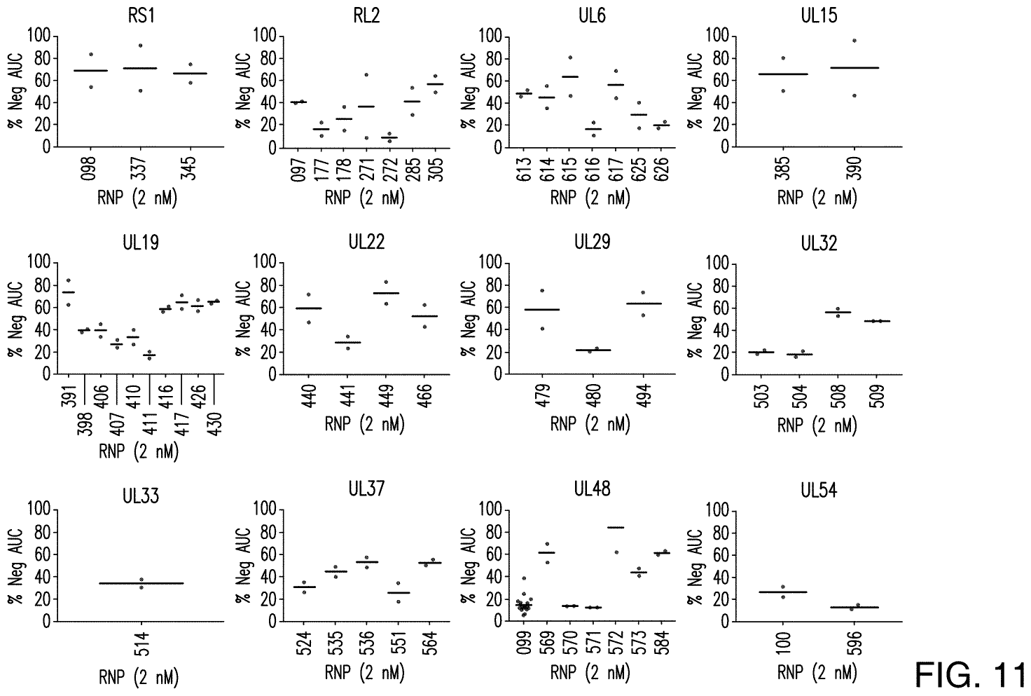

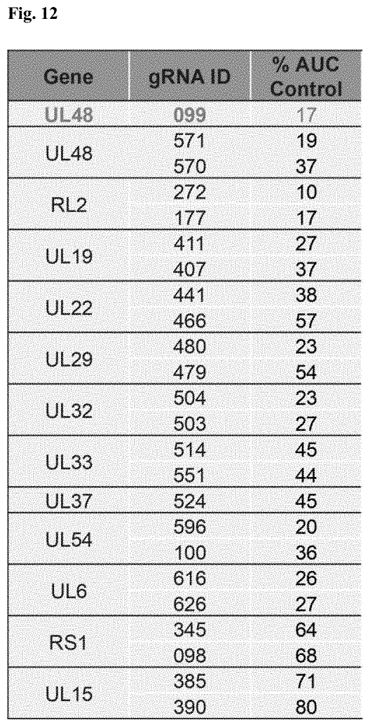

Of the 406 gRNAs screened, 51 gRNAs were selected. Additional 4 pilot gRNAs that were rationally designed were also created. The targeting domains of the 55 gRNA are set forth in SEQ ID NOs: 1-54 and in Table 2. These 55 gRNAs target 12 of 15 essential viral genes and were further screened using a virus inhibition assay. In this assay, RNPs were assembled and delivered at a fixed dose to cells cultured in vitro, which were then challenged with HSV-1. RNP-dependent effects were determined by a reduction in subsequent virus production. In particular, cells were infected over a large range of HSV-1 multiplicities of infection to best characterize RNP effect. All RNPs were then grouped into their respective essential gene. RNPs demonstrated >5-fold (>80%) reduction in HSV-1 production were selected (FIGS. 10-11). Twenty-four RNPs for 12 essential HSV-1 genes were selected based on the results of the virus inhibition assay (FIG. 12).

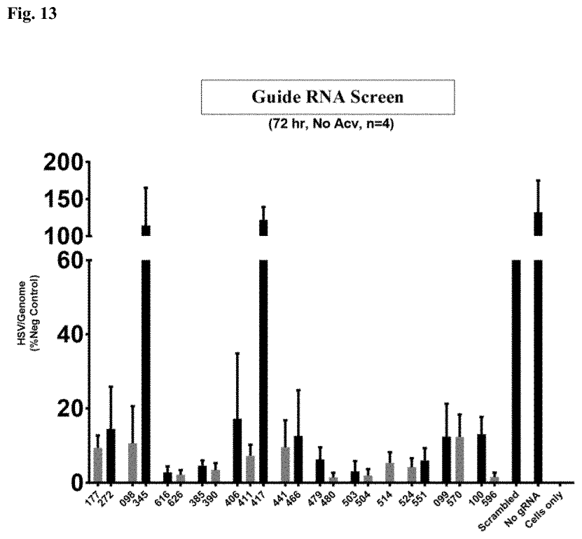

The selected 24 gRNAs were further screened using an Intracellular Bacterial Artificial Chromosome (BAC) Cutting assay. Briefly, plasmid expressing both SaCas9 and gRNAs and a BAC containing the entire HSV-1 genome were delivered to in vitro cultured cells. gRNA-directed BAC cutting occurred in the cells. After three days, HSV-1 production from the BAC was measured by ddPCR in the genomic DNA collected and isolated from the cells. Each gRNA was measured in six replicates. The best performing gRNA for each essential gene was selected based on the observed inhibition of HSV-1 production (FIG. 13). Of the selected 12 gRNAs, UL29-, UL54-, and UL32-, and UL6-targeting

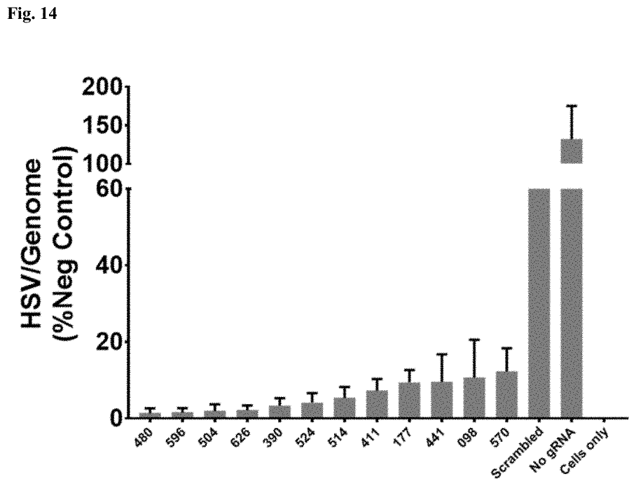

gRNAs demonstrated the greatest reduction in viral replication with respect to a scrambled gRNA (˜30-fold reduction) (FIG. 14).

Off-target effects of the selected RNP were measured by Digenome-seq. The off-target count at 100 nM for each of RNPs that was complexed with gRNA 480, 596, 504, 626, 390, 524, 514, 411, 177, 441, 098, or 570 was at or about zero. The off-target count at 1000 nM for each of RNPs that was complexed with gRNA 596, 504, 626, 390, 524, 411, or 441 was at or about zero. The off-target count at 1000 nM for RNP complexed with gRNA 480 was about 5, for RNP complexed with gRNA 514 was at or about 11, for RNP complexed with gRNA 177 was at or about 4, for RNP complexed with gRNA 098 was about 1, for RNP complexed with gRNA 570 was at or about 3.

### Example 4: Pairwise Combination (Multiplexing) gRNA Screen

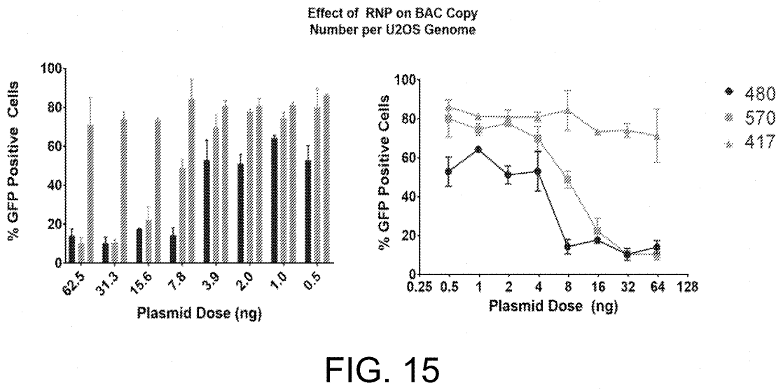

Combination gRNA screen was performed to evaluate the effects of 2, 3, 4, or 5 gRNAs in combination on GFP expression and/or the number of viral genomes per cell. Briefly, 12 guide RNAs were matrixed to cover all possible combinations, and effects on the reduction of HSV genomes per cell were demonstrated. The optimal plasmid dose for pairwise combination gRNA screen was determined (FIG. 15).

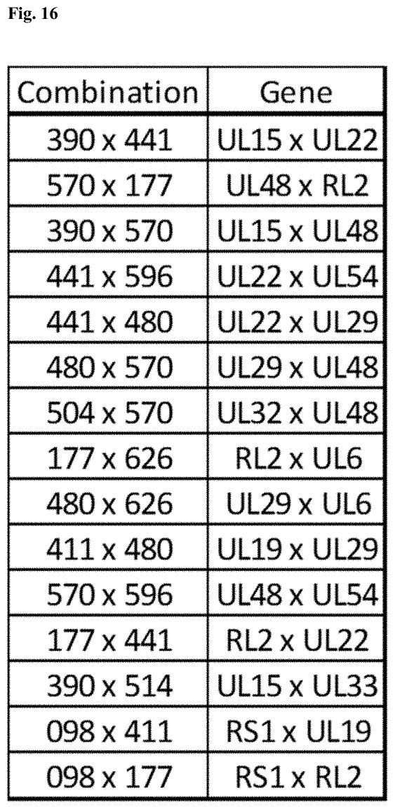

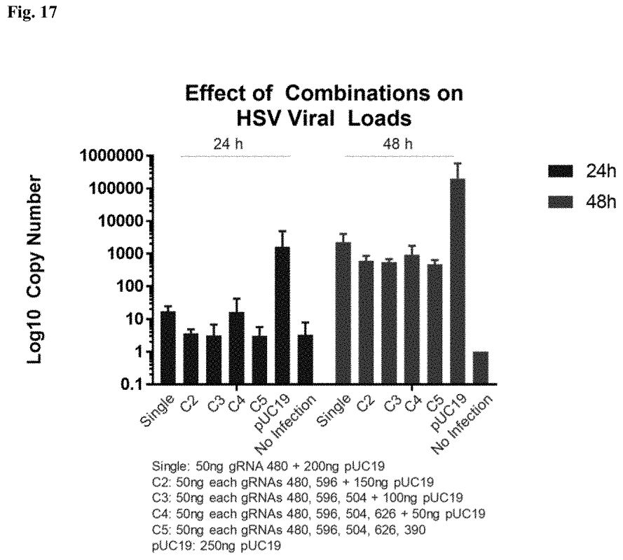

To assess multiplexing of gRNAs, Vero cells were nucleofected with plasmid expressing SaCas9 and tested gRNAs. For single gRNA, Vero cells were nucleofected with a mixture of 50 ng of Cas/gRNA plasmid and 200 ng of filler DNA. For 2-, 3-, 4-, or 5-gRNA combinations, Vero cells were transfected with a mixture 50 ng of each of the 2, 3, 4, or 5 Cas/gRNA plasmids and up to 250 ng filler DNA (e.g. for a 3-gNA combinations, 50 ng of three unique plasmids was added to 100 ng of filler DNA). Forty-eight hours after the transfection, cells were then challenged with WT HSV-1 at a MOI of 0.1. Supernatants (20 uL) were collected at 24-hour and 48-hour time points after the HSV-1 challenge. Copies of HSV genomes were measured by qPCR as previously described. HSV copy numbers were interpolated from a standard curve and replicates were averaged. Exemplary 2-gRNA combinations were show in FIG. 16. Combination gRNAs reduced HSV copy numbers by about 100-fold as compared to the scrambled gRNA (FIG. 17).

### Example 5: Selection of the Optimal Routes for Delivering AAV In Vivo

AAV1 vectors encoding an mCherry reporter transgene can be delivered to animals through corneal scarification with topical application, intrastromal injection, subconjunctival injection, and direct TG injection. In this example, these routes were evaluated for their ability of effectively transducing TGs of animals with the encoded transgenes. Female New Zealand White rabbits were used. Doses of between about 2×10¹⁴ to 2×10¹⁵ vector genomes per eye were administered to the rabbits, as allowed by the delivery routes, and shown Table 7.

TABLE 7
Administration Doses

| | | | | Volume/ | Dose/ | Total |
|---|---|---|---|---|---|---|
| | Test | | Concentration | Injection | Injection | Vol. |
| Group (n = 3) | Articles | Eyes | (vg/ml) | (ul) | (vg) | (ul) |
| Control | Buffer | Bilateral | 0 | 20 | 0 | |
| Corneal | | | | | | |
| Scarification | | | | | | |
| Corneal | AAV | Bilateral | 1 × 10¹³ | 20 | 2 × 10¹⁴ | 120 |
| Scarification | Vector | | | | | |
| Intrastromal | AAV | Bilateral | 1 × 10¹³ | 10 (4x) | 5 × 10¹⁴ | 240 |
| Injection | Vector | | | | | |
| Subconjunctival | AAV | Bilateral | 1 × 10¹³ | 200 | 2 × 10¹⁵ | 1200 |
| Injection | Vector | | | | | |
| Direct TG | AAV | Unilateral | 1 × 10¹³ | 200 | | 600-1200 |
| Injection | Vector | | | | | |

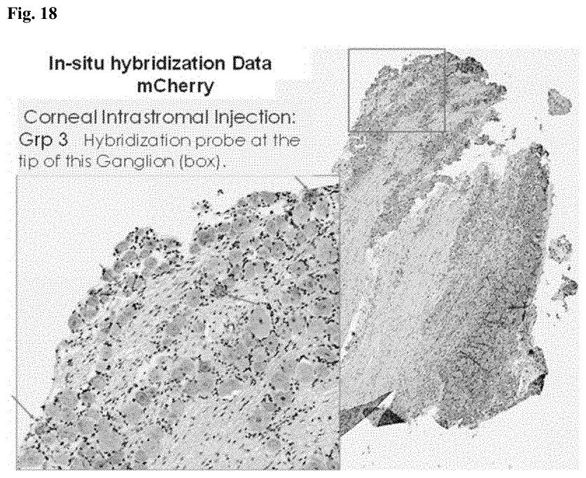

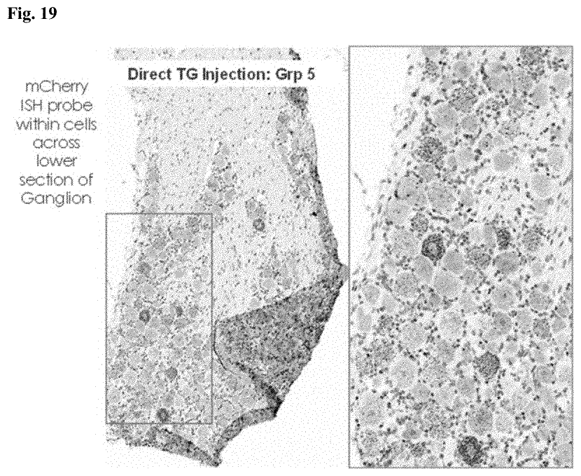

Four weeks after the administration, TGs and corneas from the rabbits were collected. AAV genomes and transgene transcripts were measured in the collected tissues by qPCR and RT-qPCR. The highest number of AAV genomes and transcript were detected in TGs of the rabbits that received the AAV vectors through direct TG injection, followed by intrastromal injection, as measured by qPCR, RT-qPCR and in situ hybridization (ISH) (FIGS. 18 & 19). AAV genomes and transcript in TGs were also increased in the subconjunctival injection group as compared to the control group in which no AAV vector was delivered. Corneal scarification delivery did not increase AAV genome and transcript abundance at TG as compared to the control group. By contrast, AAV genome abundance at cornea was the highest in the intrastromal injection group, was increased in the corneal scarification group, and was not increased in the subconjunctival injection group as compared to the control group where no AAV vector was delivered.

In sum, Intrastromal and Direct TG injection achieved significant transduction of AAV to the TGs, as verified by qPCR and ISH. Intrastromal injection provided a significant increase in AAV copy number (CN)/ug gDNA as compared to background and achieved high levels of AAV contained in the corneas. Direct TG injection provided robust and diffuse ISH signal in trigeminal ganglia while Intrastromal injection was localized to specific regions of trigeminal ganglia (FIGS. 18 & 19).

### Example 6: Assessment of Gene Editing Approach Targeting Latent HSV-1 Genomes in Rabbit Model of Recurrent HSV-1 Keratitis

Distinct AAV serotypes are compared as in vivo delivery modalities for gRNA and SaCas9 in a rabbit model of ocular HSV-1 infection. New Zealand White rabbits were first bilaterally infected with HSV-1 via corneal abrasion. After latency was established (4 weeks), infected rabbits were challenged with AAV1 or AAV8 vectors encoding both SaCas9 and dual gRNA pair UL48/RL2 expression cassettes also via corneal abrasion. After 4 weeks of vector delivery and expression, HSV-1 was induced to reactivate using epinephrine iontophoresis through rabbit corneas. Tear film swabs were collected for 12 days following viral reactivation to track viral shedding across experimental groups, after which animals' corneas and TGs were collected to assess AAV vector delivery. HSV-1 copy numbers in the TG collected from the reactivated rabbits were quantified by qPCR. Administered doses are shown in Table 8.

TABLE 8
Doses of AAV Vectors

| Treatment Groups (n = 10) | | Volume/eye | vg/eye | Target |
|---|---|---|---|---|
| Negative Ctrl | PBS | | | |
| Test Articles | AAV1-CMV- | 10 ul +10 ul | 5X10¹⁰ + | UL48 + RL2 |
| | Cas9/UL48 + RL2 | | 5X10¹⁰ | |
| | AAV8-CMV- | 10 ul +10 ul | 5X10¹⁰ + | UL48 + RL2 |
| | Cas9/UL48 + RL2 | | 5X10¹⁰ | |

| | | |
|---|---|---|
| Positive Ctrl | Acyclovir | 8 days, 200 mg/kg oral gavage |

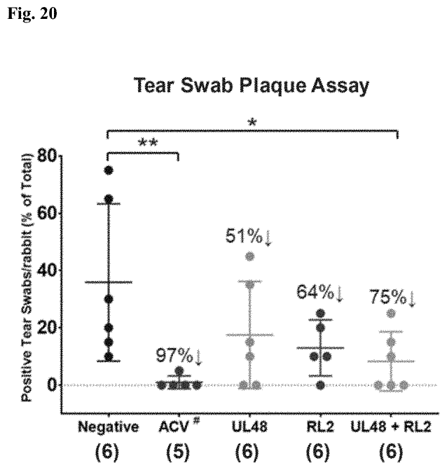

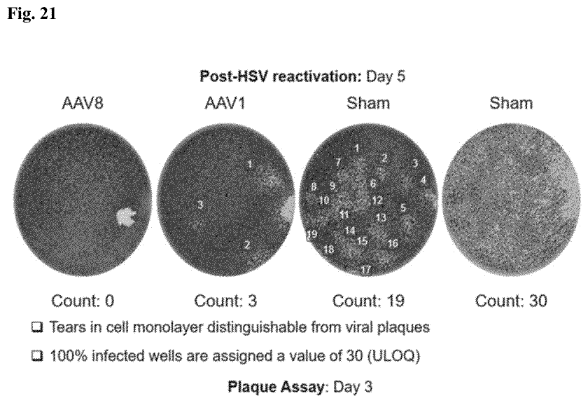

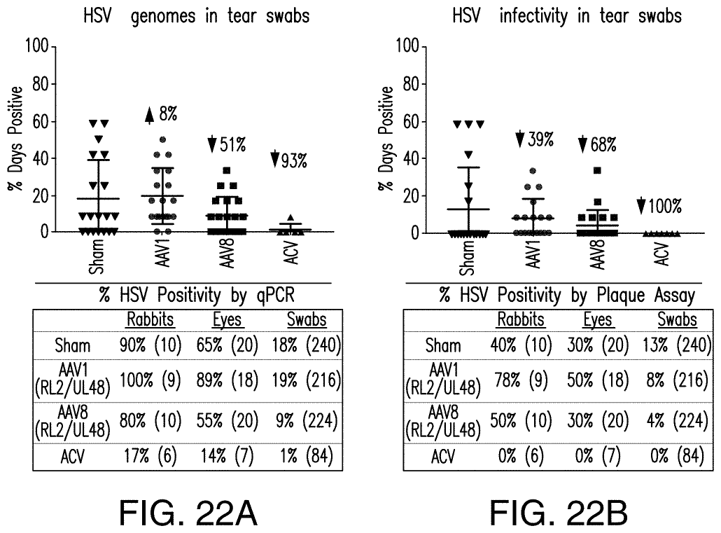

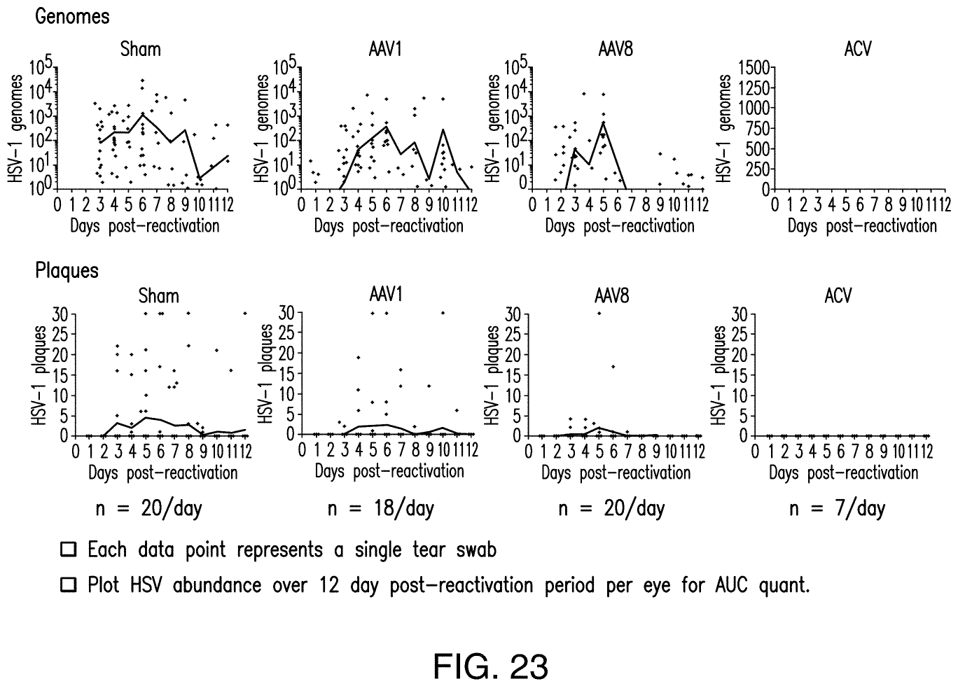

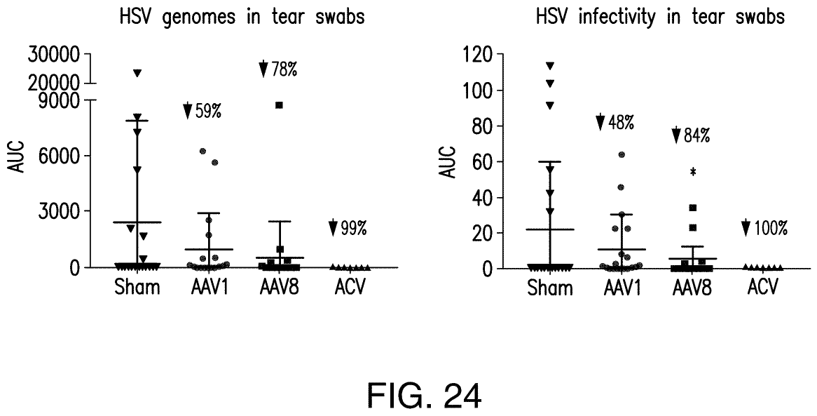

Both AAV1- and AAV8-treated animals demonstrated reduced HSV-1 loads and infectivity compared to sham-treated animals, as measured by qPCR and plaque assays of tear film swabs. AAV8-based delivery of SaCas9 and UL48/RL2 gRNA significantly reduced number of HSV-positive tear swabs over a 10-day reactivation period in infected rabbits (FIG. 20). AAV8-dependent effect was stronger than that of AAV1, as indicated by HSV genomes in tear swabs (qPCR), HSV infectivity in tear swabs (plaque assays) (FIGS. 21-24).

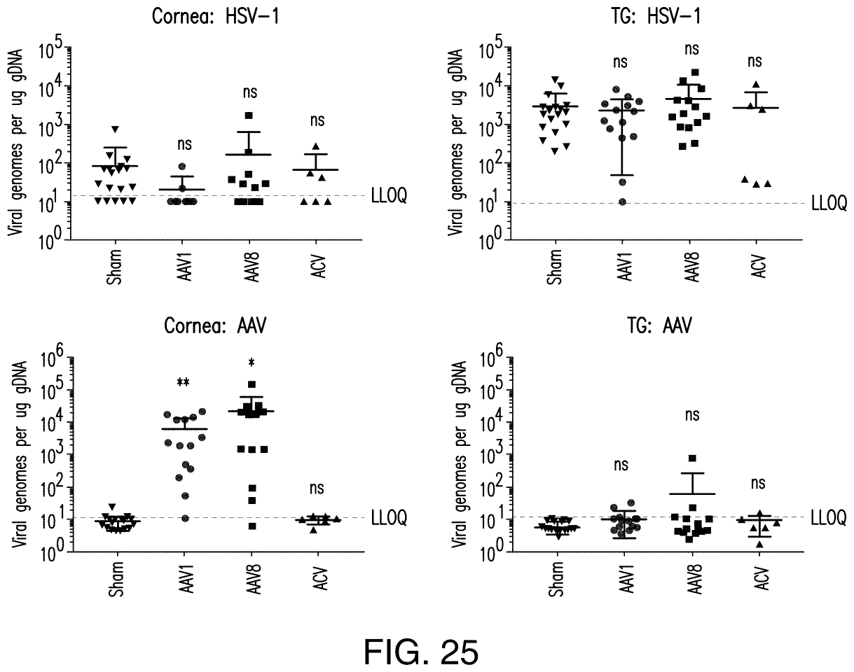

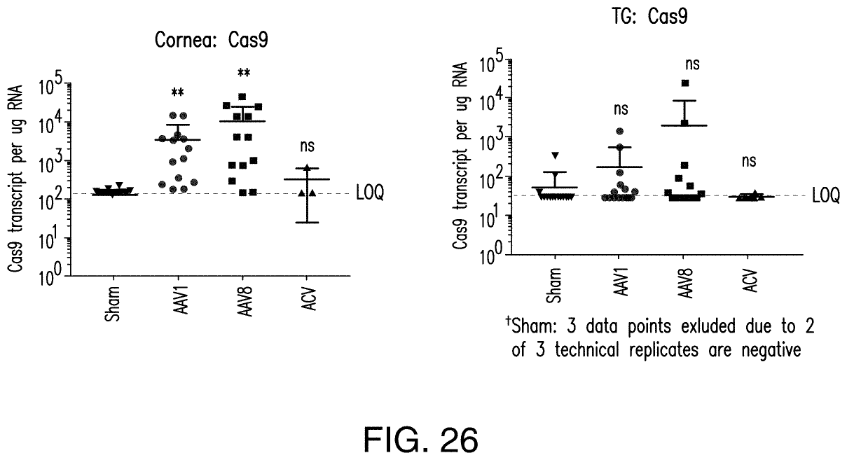

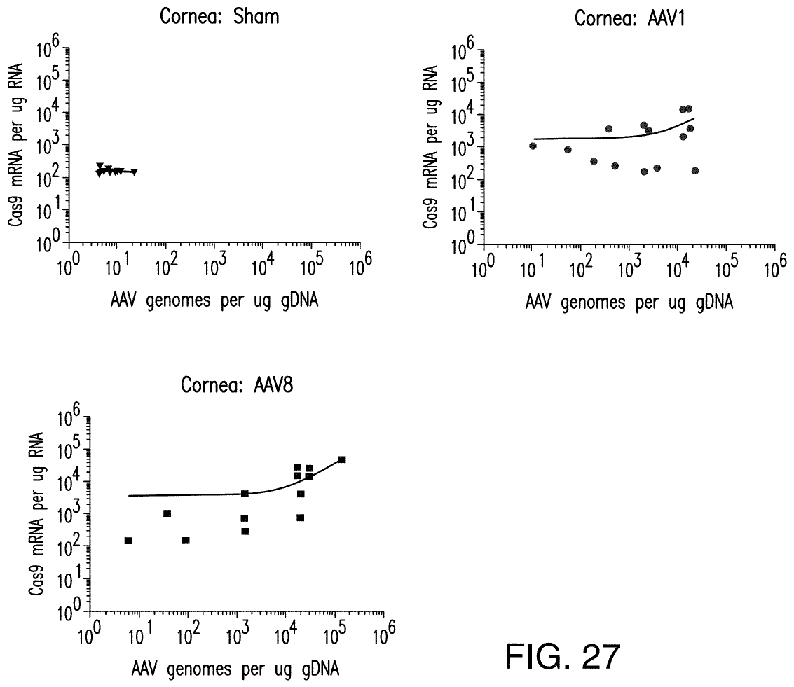

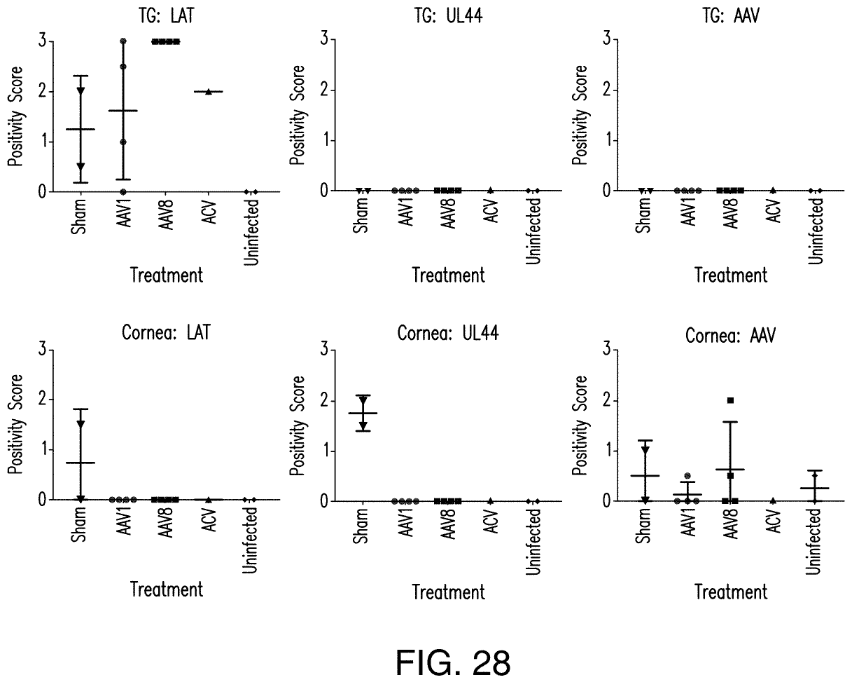

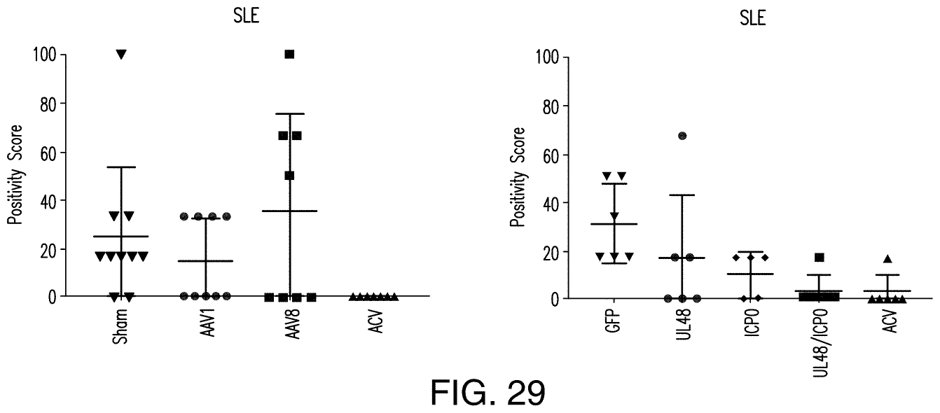

While HSV-1 was detected in animal TGs across all experimental groups, there was no significant difference in viral TG loads. HSV TG loads were consistent across treatments and AAV was detected exclusively in the corneas (FIG. 25). For both AAV serotypes, like the localization of AAV genomes, SaCas9 was expressed exclusively in corneas, but not TGs (FIG. 26). A strong positive correlation was detected between AAV8 and SaCas9 expression, which indicated better transduction efficiency of the cornea (FIG. 27). ISH-based detection of AAV and productivity of latent HSV showed that latent HSV was not detected in corneas but was detected in TGs. AAV was detected in corneas but was not detected in TGs (FIG. 28). gRNAs administration reduced the HSV-induced pathology (FIG. 29).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

What is claimed is:

1. A genome editing system comprising: (a) a first gRNA molecule comprising a first targeting domain that is complementary with a first target sequence of a first HSV-1 gene, (b) a second gRNA molecule comprising a second targeting domain that is complementary with a second target sequence of a second HSV-1 gene, and (c) an RNA-guided nuclease, wherein each of the first and second HSV-1 genes is independently selected from the group consisting of immediate early HSV-1 genes, early HSV-1 genes, and late HSV-1 genes, and wherein
i) the immediate-early HSV-1 genes are selected from the group consisting of a RL2 gene, a RS1 gene, a UL54 gene, a US1 gene, a US1.5 gene, and a US12 gene;
ii) the early HSV-1 genes are selected from the group consisting of a UL5 gene, a UL8 gene, a UL9 gene, a UL23 gene, a UL29 gene, a UL30 gene, a UL42 gene, and a UL52 gene; and/or
iii) the late HSV-1 genes are selected from the group consisting of a UL1 gene, a UL6 gene, a UL15 gene, a UL16 gene, a UL18 gene, a UL19 gene, a UL22 gene, a UL26 gene, a UL26.5 gene, a UL27 gene, a UL28 gene, a UL31 gene, a UL32 gene, a UL33 gene, a UL34 gene, a UL35 gene, a UL36 gene, a UL37 gene, a UL38 gene, a UL48 gene, a UL49.5 gene, and a US6 gene.

2. The genome editing system of claim 1, wherein
i) the immediate-early HSV-1 genes are selected from the group consisting of a RL2 gene, a RS1 gene, and a UL54 gene;
ii) the early HSV-1 gene is a UL29 gene; and/or
iii) the late HSV-1 genes are selected from the group consisting of a UL6 gene, a UL15 gene, a UL19 gene, a UL22 gene, a UL32 gene, a UL33 gene, a UL37 gene, and a UL48 gene.

3. The genome editing system of claim 2, wherein
a) the first HSV-1 gene is a late HSV-1 gene, and the second HSV-1 gene is an immediate early HSV-1 gene, an early HSV-1 gene, or a late HSV-1 gene;
b) the first HSV-1 gene is a late HSV-1 gene, and the second HSV-1 gene is an immediate early HSV-1 gene;
c) the first HSV-1 gene is an early HSV-1 gene, and the second HSV-1 gene is an early HSV-1 gene;
d) the first HSV-1 gene is an early HSV-1 gene, and the second HSV-1 gene is an immediate early HSV-1 gene;
e) the first HSV-1 gene is an immediate early HSV-1 gene, and the second HSV-1 gene is an immediate early HSV-1 gene.

4. The genome editing system of claim 1, wherein the first HSV-1 gene is a UL48 gene, and the second HSV-1 gene is a RL2 gene.

5. The genome editing system of claim 1, wherein the RNA-guided nuclease is Cas9 and
a) the first targeting domain comprises a nucleotide sequence selected from SEQ ID NOs: 1-411, and the second targeting domain comprises a nucleotide sequence selected from SEQ ID NOs: 1-411;
b) the first targeting domain comprises a nucleotide sequence selected from SEQ ID NOs: 1-54, 410, and 411, and the second targeting domain comprises a nucleotide sequence selected from SEQ ID NOs: 1-54, 410, and 411;
c) the first targeting domain comprises a nucleotide sequence selected from SEQ ID NOs: 1-25, 410, and 411, and the second targeting domain comprises a nucleotide sequence selected from SEQ ID NOs: 1-25, 410, and 411;
d) the first targeting domain comprises a nucleotide sequence selected from SEQ ID NOs: 1-14, 410, and 411, and the second targeting domain comprises a nucleotide sequence selected from SEQ ID NOs: 1-14, 410, and 411; or
e) the first targeting domain comprises the nucleotide sequence set forth in SEQ ID NO: 410, and second targeting domain comprises the nucleotide sequence set forth in SEQ ID NO: 411.

6. The genome editing system of claim 1, wherein the RNA-guided nuclease is a first Cas9 molecule, further comprising a second Cas9 molecule, both of which are configured to form complexes with the first and second gRNAs.

7. The genome editing system of claim 6, wherein at least one of the first and second Cas9 molecules comprises an S. pyogenes Cas9 molecule or an S. aureus Cas9 molecule.

8. The genome editing system of claim 6, wherein at least one of the first and second Cas9 molecules comprises a wild-type Cas9 molecule, a mutant Cas9 molecule, or a combination thereof.

9. The genome editing system of claim 6, further comprising a third gRNA molecule comprising a third targeting domain that is complementary with a third target sequence of a third HSV-1 gene, optionally a fourth gRNA molecule comprising a fourth targeting domain that is complementary with a fourth target sequence of a fourth HSV-1 gene, optionally a fifth gRNA molecule comprising a fifth targeting domain that is complementary with a fifth target sequence of a fifth HSV-1 gene.

10. A composition comprising: (a) a first gRNA molecule comprising a first targeting domain that is complementary with a first target sequence of a first HSV-1 gene, (b) a second gRNA molecule comprising a second targeting domain that is complementary with a second target sequence of a second HSV-1 gene, and (c) an RNA-guided nuclease, wherein each of the first and second HSV-1 genes is independently selected from the group consisting of immediate early HSV-1 genes, early HSV-1 genes, and late HSV-1 genes, and wherein
i) the immediate-early HSV-1 genes are selected from the group consisting of a RL2 gene, a RS1 gene, a UL54 gene, a US1 gene, a US1.5 gene, and a US12 gene;
ii) the early HSV-1 genes are selected from the group consisting of a UL5 gene, a UL8 gene, a UL9 gene, a UL23 gene, a UL29 gene, a UL30 gene, a UL42 gene, and a UL52 gene; and/or
iii) the late HSV-1 genes are selected from the group consisting of a UL1 gene, a UL6 gene, a UL15 gene, a UL16 gene, a UL18 gene, a UL19 gene, a UL22 gene, a UL26 gene, a UL26.5 gene, a UL27 gene, a UL28 gene, a UL31 gene, a UL32 gene, a UL33 gene, a UL34 gene, a UL35 gene, a UL36 gene, a UL37 gene, a UL38 gene, a UL48 gene, a UL49.5 gene, and a US6 gene.

11. The composition of claim 10, wherein
i) the immediate-early HSV-1 genes are selected from the group consisting of a RL2 gene, a RS1 gene, and a UL54 gene;
ii) the early HSV-1 gene is a UL29 gene; and/or
iii) the late HSV-1 genes are selected from the group consisting of a UL6 gene, a UL15 gene, a UL19 gene, a UL22 gene, a UL32 gene, a UL33 gene, a UL37 gene, and a UL48 gene.

12. The composition of claim 10, wherein
a) the first HSV-1 gene is a late HSV-1 gene, and the second HSV-1 gene is an immediate early HSV-1 gene, an early HSV-1 gene, or a late HSV-1 gene;
b) the first HSV-1 gene is a late HSV-1 gene, and the second HSV-1 gene is an immediate early HSV-1 gene;
c) the first HSV-1 gene is an early HSV-1 gene, and the second HSV-1 gene is an early HSV-1 gene; or
d) the first HSV-1 gene is an early HSV-1 gene, and the second HSV-1 gene is an immediate early HSV-1 gene; or
e) the first HSV-1 gene is an immediate early HSV-1 gene, and the second HSV-1 gene is an immediate early HSV-1 gene.

13. The composition of claim 10, wherein the first HSV-1 gene is a UL48 gene, and the second HSV-1 gene is a RL2 gene.

14. The composition of claim 10, wherein the RNA-guided nuclease is Cas9 and
a) the first targeting domain comprises a nucleotide sequence selected from SEQ ID NOs: 1-411, and the second targeting domain comprises a nucleotide sequence selected from SEQ ID NOs: 1-411;
b) the first targeting domain comprises a nucleotide sequence selected from SEQ ID NOs: 1-54, 410, and 411, and the second targeting domain comprises a nucleotide sequence selected from SEQ ID NOs: 1-54, 410, and 411;
c) the first targeting domain comprises a nucleotide sequence selected from SEQ ID NOs: 1-25, 410, and 411, and the second targeting domain comprises a nucleotide sequence selected from SEQ ID NOs: 1-25, 410, and 411;
d) the first targeting domain comprises a nucleotide sequence selected from SEQ ID NOs: 1-14, 410, and 411, and the second targeting domain comprises a nucleotide sequence selected from SEQ ID NOs: 1-14, 410, and 411; or
e) the first targeting domain comprises the nucleotide sequence set forth in SEQ ID NO: 410, and second targeting domain comprises the nucleotide sequence set forth in SEQ ID NO: 411.

15. The composition of claim 10, wherein the RNA-guided nuclease is a first Cas9 molecule and further comprising a second Cas9 molecule, both of which are configured to form complexes with the first and second gRNAs.

16. The composition of claim 15, wherein at least one of the first and second Cas9 molecules comprises an S. pyogenes Cas9 molecule or an S. aureus Cas9 molecule.

17. A vector comprising a polynucleotide encoding (a) a first gRNA molecule comprising a first targeting domain that is complementary with a first target sequence of a first HSV-1 gene, (b) a second gRNA molecule comprising a second targeting domain that is complementary with a second target sequence of a second HSV-1 gene, and (c) an RNA-guided nuclease, wherein each of the first and second HSV-1 genes is independently selected from the group consisting of immediate early HSV-1 genes, early HSV-1 genes, and late HSV-1 genes, and wherein
i) the immediate-early HSV-1 genes are selected from the group consisting of a RL2 gene, a RS1 gene, a UL54 gene, a US1 gene, a US1.5 gene, and a US12 gene;
ii) the early HSV-1 genes are selected from the group consisting of a UL5 gene, a UL8 gene, a UL9 gene, a UL23 gene, a UL29 gene, a UL30 gene, a UL42 gene, and a UL52 gene; and/or
iii) the late HSV-1 genes are selected from the group consisting of a UL1 gene, a UL6 gene, a UL15 gene, a UL16 gene, a UL18 gene, a UL19 gene, a UL22 gene, a UL26 gene, a UL26.5 gene, a UL27 gene, a UL28 gene, a UL31 gene, a UL32 gene, a UL33 gene, a UL34 gene, a UL35 gene, a UL36 gene, a UL37 gene, a UL38 gene, a UL48 gene, a UL49.5 gene, and a US6 gene.

18. The vector of claim 17, wherein
i) the immediate-early HSV-1 genes are selected from the group consisting of a RL2 gene, a RS1 gene, and a UL54 gene;
ii) the early HSV-1 gene is a UL29 gene; and/or
iii) the late HSV-1 genes are selected from the group consisting of a UL6 gene, a UL15 gene, a UL19 gene, a UL22 gene, a UL32 gene, a UL33 gene, a UL37 gene, and a UL48 gene.

19. The vector of claim 18, wherein
a) the first HSV-1 gene is a late HSV-1 gene, and the second HSV-1 gene is an immediate early HSV-1 gene, an early HSV-1 gene, or a late HSV-1 gene;
b) the first HSV-1 gene is a late HSV-1 gene, and the second HSV-1 gene is an immediate early HSV-1 gene;
c) the first HSV-1 gene is an early HSV-1 gene, and the second HSV-1 gene is an early HSV-1 gene;
d) the first HSV-1 gene is an early HSV-1 gene, and the second HSV-1 gene is an immediate early HSV-1 gene; or
e) the first HSV-1 gene is an immediate early HSV-1 gene, and the second HSV-1 gene is an immediate early HSV-1 gene.

20. The vector of claim 17, wherein the first HSV-1 gene is a UL48 gene, and the second HSV-1 gene is a RL2 gene.

21. The vector of claim 17, wherein the RNA-guided nuclease is Cas9 and
a) the first targeting domain comprises a nucleotide sequence selected from SEQ ID NOs: 1-411, and the second targeting domain comprises a nucleotide sequence selected from SEQ ID NOs: 1-411;
b) the first targeting domain comprises a nucleotide sequence selected from SEQ ID NOs: 1-54, 410, and 411, and the second targeting domain comprises a nucleotide sequence selected from SEQ ID NOs: 1-54, 410, and 411;
c) the first targeting domain comprises a nucleotide sequence selected from SEQ ID NOs: 1-25, 410, and 411, and the second targeting domain comprises a nucleotide sequence selected from SEQ ID NOs: 1-25, 410, and 411;
d) the first targeting domain comprises a nucleotide sequence selected from SEQ ID NOs: 1-14, 410, and 411, and the second targeting domain comprises a nucleotide sequence selected from SEQ ID NOs: 1-14, 410, and 411; or
e) the first targeting domain comprises the nucleotide sequence set forth in SEQ ID NO: 410, and second targeting domain comprises the nucleotide sequence set forth in SEQ ID NO: 411.

22. The vector of claim 17, wherein the RNA-guided nuclease is a first Cas9 molecule and further comprising a second Cas9 molecule, both of which are configured to form complexes with the first and second gRNAs.

23. The vector of claim 22, wherein at least one of the first and second Cas9 molecules comprises an S. pyogenes Cas9 molecule or an S. aureus Cas9 molecule.

24. The vector of claim 17, wherein, the vector is a viral vector.

25. The vector of claim 17, wherein the vector is an Adeno-associated virus (AAV) vector.

26. The vector of claim 25, wherein the AAV vector is a serotype 1, 2, 3, 4, 5, 6, 7, 8 or 9 vector.

27. A method of altering a first HSV-1 gene and a second HSV-1 gene in a cell, comprising administrating to the cell a genome editing system of claim 1.

28. A method for treating and/or preventing a HSV-related disease in a subject, comprising administrating to the subject a genome editing system of claim 1
